(19) United States
(12) Patent Application Publication
(10) Pub. No.: US 2025/0345356 A1
(43) Pub. Date: Nov. 13, 2025

(54) TIRAPAZAMINE COMPOSITIONS AND METHODS

(71) Applicant: Teclison, Inc., Princeton, NJ (US)

(72) Inventor: Ruey-min Lee, Short Hills, NJ (US)

(73) Assignee: Teclison, Inc., Princeton, NJ (US)

(21) Appl. No.: 19/171,662

(22) Filed: Apr. 7, 2025

Related U.S. Application Data
(60) Provisional application No. 63/643,695, filed on May 7, 2024.

(51) Int. Cl. A61K 31/724; A61K 31/53; A61P 35/00

(52) U.S. Cl. CPC A61K 31/724; A61K 31/53; A61P 35/00

(57) ABSTRACT

The present disclosure provides cyclodextrin inclusion complexes of a β-cyclodextrin substituted host molecule wherein the guest is tirapazamine. The molar ratio of the tirapazamine guest to the cyclodextrin host ranges from about 14:1 to about 2:1, inclusive. The complexed tirapazamine has advantageous properties when compared to non-complexed tirapazamine in that the tirapazamine complex is water soluble and, at a molar ratio of the β-cyclodextrin substituted host molecule to the tirapazamine guest of 2:1, the pH of a 0.7-1 mg/mL solution of the inclusion complexes containing tirapazamine ranges from about pH 5.3 to about pH 6.4. The present disclosure also provides pharmaceutical compositions comprising a pharmaceutically acceptable carrier and cyclodextrin inclusion complexes of β-cyclodextrin substituted host molecules wherein the guest is tirapazamine. The pharmaceutical composition comprising the β-cyclodextrin-complexed tirapazamine demonstrates improved stability, improved solubility and reduced toxicity of the tirapazamine compared to non-complexed tirapazamine alone.

### CROSS-REFERENCE TO RELATED APPLICATIONS

The present application claims the benefit of priority to U.S. provisional patent application 63/643,695, entitled “Tirapazamine compositions and methods,” which was filed May 7, 2024, the content of which are incorporated herein by reference in its entirety.

### FIELD OF THE INVENTION

The described invention relates to formulations of tirapazamine (TPZ), more particularly to cyclodextrin inclusion complexes containing TPZ.

### BACKGROUND OF THE INVENTION

A tumor originates from a normal cell that has undergone tumorigenic transformation. This transformed cell is the cell-of-origin (“COO”) for the tumor. Tumorigenesis consists of four stages [Bi, Q. J. Immunology Res. (2022) (2022) article 3128933, citing Balani, S., et al. Nature Communic. (2017) 8 (1): article 15422; Chaffer, C L and Weinberg, RA. Cancer Discovery (2015) 5 (10): 22-24; Loeb, LA and Harris, CC. Cancer Res. (2008) 68 (17): 6863-6871]: (a) tumor initiation, the initial stage of tumorigenesis, is the stage in which normal cells undergo irreversible genetic alterations under the response of oncogenic factors, thus transforming into COOs with the possibility of malignant transformation; (b) tumor promotion is the period during which COOs clone selectively and transform into premalignant cells under the influence of protumor factors and other specific conditions; (c) malignant conversion is the stage in which premalignant cells start expressing malignant phenotypes; and (d) tumor progression is the final stage of tumorigenesis, in which premalignant cells develop into real tumor cells, obtain a series of new biological characteristics (including sustaining proliferative signaling, evading growth suppressors, resisting cell death, enabling replicative immortality, inducing or accessing vasculature, activating invasion and metastasis, deregulating cellular metabolism, avoiding immune destruction, and unlocking phenotypic plasticity, nonmutational epigenetic reprogramming, polymorphic microbiomes, and senescent cells) [Id., citing Hanahan, D. Cancer Discovery (2022) 12 (1): 31-46], and undergo more invasion and metastasis. These characteristics are the result of the superimposition of various factors, particularly the tumor microenvironment (TME).

### Hepatocellular Carcinoma

Hepatocellular carcinoma (HCC), the leading type of primary liver cancer and a significant global health burden, is a solid tumor with a high degree of capillarization and arterialization. [Yao, C., et al. Cancer Giol. Med. (2023) 20 (1): 25-43]. HCC ranks as the third leading cause of cancer-related deaths worldwide, with its incidence and mortality rates on the rise [Argentiero, A. et al. J. Clinical Med. (2023) 12: 7469, citing Fitzmaurice, C., et al. JAMA Oncol. (2017) 3: 1683-1691]. The increasing prevalence of HCC can be attributed to various factors, including the growing prevalence of chronic liver diseases, such as cirrhosis, hepatitis B and hepatitis C infections, and nonalcoholic fatty liver disease (NAFLD) [Id., citing Forner, A., et al. Lancet (2018) 391: 1301-1314].

### Tumor Microenvironment (TME)

The tumor microenvironment (TME) in HCC consists of a complex network of cellular and non-cellular components that interact dynamically to shape the behavior and progression of tumors that play a critical role in tumor growth, invasion, metastasis, and therapeutic resistance.

### Cellular Components.

Cancer-Associated Fibroblasts (CAFs) Cancer-associated fibroblasts (CAFs), which are activated fibroblasts that have acquired distinct characteristics and functions in response to signals from cancer cells and the TME, are the most abundant cell type in the HCC tumor TME and play a crucial role in tumor progression and metastasis. CAFs. They secrete various factors, including growth factors, cytokines, and extracellular matrix (ECM) proteins, which promote tumor cell proliferation, angiogenesis, immune suppression, and therapeutic resistance in HCC [Id., citing Kalluri, R. Nat. Rev. Cancer (2016) 16: 582-598; Mueller, S N and Germain, RN. Nat. Rev. Immunol. (2009) 9: 618-629; Kubo, N., et al. World J. Gastroenterol. (2016) 22: 6841-6850]. CAFs contribute to the remodeling of the ECM, creating a supportive niche for tumor growth and invasion [Id., citing Kallluri, R. Nat. Rev. Cancer (2016) 582-598]. The extracellular matrix (ECM), which is mainly secreted by cancer-associated fibroblasts (CAFs), which produce more ECM proteins than normal fibroblasts, is composed of various macromolecules, including collagens, glycoproteins (fibronectin and laminins), proteoglycans and polysaccharides with different physical and biological properties. [Brassart-Pasco, S., et al. Front. Oncology (2020) 10: 397]. Interstitial matrix, primarily synthesized by stromal cells, is rich in fibrillary collagens and proteoglycans. CAF secretome analyses show an increased secretion of bone morphogenetic protein (BMP)-1, thrombospondin-1 and elastin interface 2 [Id., citing Santi, A., et al. Proteomics (2018) 18: e1700167; Socovich, A M and Naba, A. Semin. Cell Dev. Biol. (2019) 89: 157-166].

CAFs interact with other cell types within the TME, such as immune cells and endothelial cells, through paracrine signaling and direct cell-cell contact, further facilitating tumor progression and metastasis [Argentiero, A., et al. J. Clinical Med. (2023) 12: 7469, citing Kalluri, R. Nat. Rev. Cancer (2016) 16: 582-598; Mueller, S N and Germain, RN. Nat. Rev. Immunol. (2009) 9: 618-29]. They also play a role in drug resistance: CAF-derived and secreted phosphoprotein 1 (SPP1) enhances tyrosine-kinase inhibitor resistance by activating alternative oncogenic signals and promoting epithelial-to-mesenchymal transition.

Immune cells. The immune response within the HCC TME is dysregulated, leading to immune evasion and tumor progression. Various immune cell populations have been identified in the HCC TME, including tumor-associated macrophages (TAMs), myeloid-derived suppressor cells (MDSCs), and regulatory T-cells (Tregs).

Tumor-Associated Macrophages (TAMs). TAMs are key regulators of the immune response in HCC. They exhibit a distinct polarization toward an M2-like phenotype, characterized by the secretion of anti-inflammatory cytokines and growth factors, such as interleukin-10 (IL-10) and transforming growth factor-beta (TGF-β), promoting angiogenesis, tissue remodeling, and immune suppression [Id., citing Zhang, Q., et al. Cell (2019) 179: 829-845]. TAMs also inhibit T-cell activation and function through the secretion of inhibitory molecules, including programmed death-ligand 1 (PD-L1), thereby contributing to immune evasion in HCC [Id., citing Zheng, H., et al. (2023) 9: 65].

Myeloid-derived suppressor cells (MDSCs). MDSCs, a heterogeneous population of immature myeloid cells with immunosuppressive properties, contribute to immune suppression in HCC. MDSCs inhibit T-cell responses through various mechanisms, such as the production of arginase-1 and inducible nitric oxide synthase (iNOS), leading to the depletion of essential nutrients and the generation of reactive oxygen species (ROS) [Id., citing Fu, J., et al. Gastroenterology (2007) 132: 2328-2339]. This inhibitory environment hampers effective antitumor immune responses and promotes tumor progression.

Regulatory T cells (Tregs). Tregs are a specialized subset of CD4+ T cells that play a critical role in maintaining immune homeostasis and in preventing excessive immune responses. In the HCC TME, Tregs accumulate and exert their suppressive effects by inhibiting effector T-cell responses and promoting tolerance to tumor antigens [Id., citing Fu, J., et al. Gastroenterology (2007) 132: 2328-2339]. The presence of Tregs in the TME has been associated with poor prognosis and reduced survival in HCC patients.

Non-parenchymal liver cells. Liver is an immune organ with a number of immunocompetent cells. Non-parenchymal resident cells, such as Kupffer cells, hepatic stellate cells (HSC), and liver sinusoidal endothelial cells (LSEC), cooperate in the maintenance of immune tolerance.

Kupffer cells are liver-resident macrophages that act as antigen-presenting cells (APC) to form the first line of defense against pathogens [Chen, C., et al. Front. Immunology (2023) 14: 1133308, citing Ebrahimimkhani, M R, et al. Hepatol. (Baltimore, MD) (2011) 54 (4): 1379-1387; Keenan, B P, et al. J. Immunotherapy Cancer (2019) 7 (1): 267]. They can contribute to hepatocarcinogenesis and immune escape by several mechanisms: 1) secretion of immunosuppressive cytokines (e.g., IL-10) [Id., citing Knolle, P. et al. J. Hepatol. (1995) 22 (2): 226-229]; 2) upregulation of inhibitory immune checkpoint ligand PD-1 [Id., citing Heymann, F. et al. Hepatol. (Baltimore MD) (2015) 62 (1): 279-291]; 3) downregulation of costimulatory molecules (CD80 and CD86) [Id., citing Ringelban, M., et al. Nat. Immunol. (2018) 19 (3): 222-232; Hou, J., et al. J. Hepatol. (2020) 72 (1): 167-182]; 4) production of Indoleamine 2-3 dioxygenase (IDO) [Id., citing Yan, M L, et al. World J. Gastroenterol. (2010) 16 (5): 636-640; and 5) recruitment of Treg cells and of T helper 17 (TH17) cells [Id., citing Ringelban, M., et al. Nat. Immunol. (2018) 19 (30): 222-232; Heymann, F., et al. Hepatol. (1995) 22 (20): 226-229; Hou, J., et al. J. Hepatol. (2020) 72 (1): 167-182]. The interaction of PD-L1 expressed by Kupffer cells and PD-1 expressed by T cells leads to T-cell exhaustion in human HCC [Id., citing Wu, K., et al. Cancer Res. (2009) 69 (20): 8067-875].

HSCs can secrete hepatocyte growth factor (HGF) that enables MDSC and Treg cells to accumulate inside the liver [Id., citing Hochst, B., et al. J. Hepatol. (2013) 59 (30: 528-535]. Also, HSCs express high levels of PD-L1 to induce T cell apoptosis [Id., citing Dunham, R M, et al. J. Immunol. (Baltimore MD 1950) (2013) 190 (5): 2009-2016]. HSCs can transdifferentiate into CAFs and consequently promote angiogenesis. [Yao, C., et al. Cancer Biol. Med. (2023) 20 (1): 25-43].

LSECs, which line the low shear, sinusoidal capillary channels of the liver and are the most abundant non-parenchymal hepatic cell population, have a critical role in maintaining immune homeostasis within the liver and in mediating the immune response during acute and chronic liver injury. LSECs have potent scavenger capabilities by virtue of their expression of many scavenger receptors, including mannose receptor (MR), CD32, stabilin 1, stabilin 2, scavenger receptor B1 (SRB1) and scavenger receptor class F member 1 (SCARF 1), liver/lymph node-specific ICAM3-grabbing non-integrin (LSIGN), lymphatic vessel endothelial hyaluronic acid receptor 1 (LYVE1) and pro-LDL receptor-related protein 1 (LRP1). Scavenger receptors are a diverse family of pattern recognition receptors that, like TLRs, are highly evolutionarily conserved. The high levels of scavenger receptors on LSECs give them a high endocytic capacity. LSECs constitutively express low levels of intercellular adhesion molecule 1 (ICAM1), ICAM2 and vascular cell adhesion protein 1 (VCAM1). Minimal chemokine expression is seen in unstimulated LSECs, although they will express factors such as CXC-chemokine ligand 9 (CXCL9)-CXCL11, CC-chemokine ligand 25 (CCL25), CX3C-chemokine ligand 1 (CX3CL1) and CXCL16 in response to cytokine stimulation. They can also present chemokines derived from neighboring or underlying cells to promote binding and migration of immune cell subsets. In addition to their roles in pathogen recognition and as antigen-presenting cells, LSECs also have a critical role in regulating the recruitment of leukocytes into liver tissue. LSECs play a role in the quiescence of HSCs, which is lost during capillarization of LSECs, which permits HSC activation and fibrogenesis. [Id., citing Shetty, S., et al. Nat. Rev. Gastroenterol. Hepatol. (2018) 15 (9): 555-567].

During cirrhosis and chronic hepatitis, LSECs can undergo capillarization, which is mechanistically linked to the development of chronic inflammatory disease. [Shetty, S., et al. Nat. Rev. Gastroenterol. Hepatol. (2018) 15 (9): 555-567, citing Couvelard, A., et al. Am. J. Pathol. (1993) 143: 738-752]. In rodent models, capillarization is associated with enhanced antigen presentation and cytotoxic T cell priming during fibrosis [Id., citing Connolly, M K, et al. J. Immunol. (2010) 185: 2200-2208], and in nonalcoholic steatohepatitis (NASH), capillarization precedes and contributes to the transition from simple steatosis to steatohepatitis [Id., citing Miyao, M., et al. Lab Invest. (2015) 95: 1130-1144].

The changes that occur in LSECs in response to chronic inflammation also affect angiogenic pathways. Neo-angiogenesis is a key feature of chronic liver disease; the majority of neo-vessels arise from portal vein branches and are closely associated with areas of fibrogenesis [Id., citing Onori, P., et al. J. Hepatol. (2000) 33: 555-563; Fernandez, M., et al. J. Hepatol. (2009) 50: 604-620]. A key initiating step is the capillarization of LSECs, which leads to increased hepatocyte hypoxia and subsequent release of pro-angiogenic factors [Id., citing Corpechot, C., et al. Hepatology (2002) 35: 1010-1021; Rosmorduc, O., et al. Am. J. Pathol. (1999) 155: 1065-1073]. The LSEC response is context-specific; for example, acute injury can induce CXCR7 expression and a regenerative response, whereas chronic injury leads to CXCR4 induction, HSC proliferation and fibrogenesis [Id., citing Ding, B S, et al. Nature (2014) 505: 97-102]. During ischemia-reperfusion injury, LSECs develop a pro-inflammatory, prothrombotic phenotype associated with vasoconstriction [Id., citing Peralta, C., et al. J. Hepatol. (2013) 59: 1094-1106]. These changes have been directly linked to neutrophils because IL-33 released by LSECs during ischemia-reperfusion injury triggers the release of neutrophil extracellular traps (NETs), which exacerbate acute hepatic injury [Id., citing Yazdani, H O, et al. J. Hepatol. (2017) 678: 130-139]. In chronic injury, the changes in endothelial phenotype that accompany capillarization and precede fibrosis have been linked to alterations in signaling via the Hedgehog gene family [Id., citing Xie, G., et al. Gut (2013) 62: 299-309] and lead to vasoconstriction and increased intrahepatic vascular resistance due to reduced nitric oxide production by LSECs [Id., citing Rockey, D C and Chung J J. Gstroenterology (1998) 114: 344-351]. Tumor progression in hepatocellular carcinoma is associated with changes in the phenotype of peritumoral LSECs and increased production of angiogenic factors including IL-6 [Id., citing Zhang, P Y et al. BMC Cancer (2015) 15: 830; Geraud, C., et al. Liver Int. (2013) 33: 1428-1440].

CAFs can trigger NK cell dysfunction by secreting prostaglandin E2 (PGE2) and IDO, and prompt MDSC production by releasing IL-16 and CXCL12 [Id., citing Deng, Y, et al. Oncogene (2017) 36 (8): 1090-1101].

### Non-Cellular Components

Extracellular Matrix (ECM). The ECM is a complex network of proteins and polysaccharides that provides structural and biochemical support to cells within the TME. In HCC, the ECM undergoes dynamic changes that promote tumor growth, invasion, and metastasis. Alterations in the composition of ECM, remodeling enzymes, and stiffness affect cellular behaviors, such as cell adhesion, migration, and signaling pathways that are involved in tumor progression [Id., citing Winkler, J., et al. Nat. Commun. (2020) 11: 5120]. The dysregulated ECM in HCC contributes to the invasive and metastatic behavior of tumor cells by providing a physical scaffolding and modulating cellular signaling events. Additionally, the abnormal ECM can create a barrier that limits the penetration and efficacy of therapeutic agents.

Hypoxia and Angiogenesis. Angiogenesis in HCC is robustly stimulated by hypoxia. [Yao, C., et al. Cancer Biol. Med. (2023) 20 (1): 25-43]. It arises due to the rapid proliferation of tumor cells, insufficient vascularization, and the abnormal architecture of tumor blood vessels. Hypoxia develops within the solid tumors, because of the high interstitial pressure and the distance between the tumor cells and adjacent capillaries, Pro-angiogenic factors (e.g., vascular endothelial growth factors (VEGFs), platelet derived growth factors (PDGFs), fibroblast growth factors (FGFs) and angiopoietins) stimulate the proliferation and migration of ECs from the vessels in the surrounding tissues. [Id.] Several cytokines also play a role in tumor angiogenesis. [Id.]

Hypoxia as a hallmark of the TME presents in the majority of tumors and arises from an imbalance between increased oxygen consumption and inadequate oxygen supply. Although the rapid proliferation of tumors can stimulate the growth of new vasculature and tumor-induced angiogenesis leads to the unorganized growth of vasculature, the precisely distributed vasculature in normal tissues contributes to the delivery of oxygenated blood. In contrast, the irregular distribution of tumor vasculature caused by persistent hypoxic conditions can result in an increase in the distance between the capillaries, exceeding the capacity of oxygen to diffuse [Jing, X, et al. Molecular Cancer (2019) 18: 157, citing Wigerup, C. et al. Pharmacol. Ther. (2016) 164: 152-169; Wilson, W R and Hay, MP. Nat. Rev. Cancer (2011) 11: 393-410]. Such chronic hypoxia or diffusion-restricted hypoxia causes the necrosis of tumor cells within the 180-μm periphery of blood vessels. Current anticancer strategies target only tumor cells around the blood vessels rather than those in poorly perfused regions [Id., citing Loeges, S., et al. Cancer Cell (2009) 15: 167-170; Minchinton, A I and Tannock, I F. Nat. Rev. Cancer (2006) 6: 583-592].

Hypoxia induces changes in gene expression and subsequent proteomic changes that have many important effects on various cellular and physiological functions, ultimately limiting patient prognosis [Jing, X., et al. Molecular Cancer (2019) 18: 157, citing Roma-Rodrigues, C., et al. Intl J. Mol. Sci. (2019) 20]. For example, slowly dividing cells in hypoxic regions can escape most of the cytotoxic drugs that target rapidly dividing cells, and cancer stem cells may also be present in poorly hypoxic regions ensuring epithelial-to-mesenchymal transition (EMT) [Birner, P. etal. Cancer Res. (2000) 60: 4693-4696]. Hypoxia also generates intratumoral oxygen gradients, contributing to the plasticity and heterogeneity of tumors and promoting a more aggressive and metastatic phenotype.

Under hypoxic conditions, hypoxia-inducible factors (HIFs), particularly HIF-1α and HIF-2α, are stabilized and translocated to the nucleus, where they activate the expression of genes involved in angiogenesis, glycolysis, and cell survival [Argentiero, A., et al. J. Clinical Med. (2023) 12: 7469, citing Guo, Y et al. Oncol. Rep. (2020) 43: 3-15]. In HCC, hypoxia-induced HIF activation promotes the secretion of pro-angiogenic factors, including vascular endothelial growth factor (VEGF), platelet-derived growth factor (PDGF), and angiopoietin-2 (Ang-2), which stimulate the formation of new blood vessels and the recruitment of endothelial cells [Villa, E., et al. Gut (2016) 65: 861-869]. This hypoxia-driven angiogenic response supports tumor growth, provides nutrients and oxygen to tumor cells, and facilitates metastasis by promoting the formation of abnormal and leaky blood vessels.

Hypoxia causes vascular leakage and abnormal lymphatic drainage in the tumor, leading to an increase in interstitial fluid pressure [Jing, citing Nelson, D A, et al. Genes Dev. (2004) 18: 2095-2107]. To adapt to low levels of oxygen and nutrients, tumor cells develop new blood vessels by de novo angiogenesis. However, such newly formed blood vessels are leaky because of their discontinuous endothelium, and, along with the obstruction of lymphatic drainage, produces vascular hyperpermeability and enhanced permeation [Id., citing Maeda, H., et al. J. Control Release (2000) 65: 271-284].

### Hypoxia-Inducing Factor

Hypoxia-inducible factor (HIF) is a heterodimer composed of two basic helix-loop-helix proteins of the Per-ARNT-Sim (PAS) family: an oxygen-sensitive α-subunit and a constitutively expressed β-subunit [Id., citing Semenza, GL. Nat. Rev. Cancer (2003) 3: 721-732]. Three HIF-α isoforms have been identified in mammals. When compared with HIF-1, a transcriptional nucleoprotein with a wide range of target genes, HIF-2 seems to be more restricted in expression in the tissue and less is known about HIF-3 [Id., citing Wiesener, M S et al. FASEB J.: 271) 17: 271-273]. HIFs play a distinct role in tumorigenesis, and immunohistochemical analyses show that HIF-1α and HIF-2 α are overexpressed in the majority of human cancers.

Under normoxic conditions, two critical proline residues in HIF-α subunits are subject to hydroxylation within their oxygen-dependent degradation domain by enzymes called HIF prolyl hydroxylase domain family proteins (PHDs), which use oxygen, ferrous iron, and α-ketoglutarate as substrates. PHDs are HIF-preserved hydroxylases found in mammals, with three subtypes, PHD1, PHD2, and PHD3, as regulators of HIF-1α oxygen sensors to participate in the degradation of HIF-1α. PHD2 keeps HIF-1α at a stable low level in an anoxic environment as the main rate-limiting enzyme, and its activity is controlled mainly by the intracellular oxygen concentration. Then, the von Hippel-Lindau tumor suppressor protein (pVHL) interacts with HIF-αas a result of hydroxylation and recruits an E3 ubiquitin ligase complex, resulting in ubiquitination and subsequent proteasomal degradation of HIF-α.

Hypoxia induces a number of complex intracellular signaling pathways, such as the major HIF pathway, the PI3K/AKT/mTOR pathway [Muz, B., et al. Hypoxia (Auk) (2015) 3: 83-92, citing Agani, F. and Jiang, B H. Curr. Cancer Drug Targets (2013) 13 (3): 245-251; Courtnay, R, et al. Mol. Biol. Rep. (2015) 42 (4): 841-851], the MAPK/ERK pathways [Id., citing Seta, K A, et al., Sci STKE (2002) 2002 (146): rel1; Sanchez, A., et al. J. Alzheimers Dis. (2012) 32 (3): 587-597; Minet, E., et al. FEBS Lett. (2000) 468 (1): 53-58] and NFκB signaling pathways [Id., citing Koong, A C, et al. Cancer Res. (1994) 54 (6): 1425-1430]. These pathways are involved in cell proliferation, survival, apoptosis, metabolism, migration and inflammation.

Under hypoxic conditions, HIF-1α mediates hypoxia-induced signaling, which plays a role in multiple steps of the transfer cascade [Jing, citing Semenza, GL. Annu. Rev. Pathol. (2014) 9: 47-71]. The inhibitory hydroxylation of HIF-α is reduced, leading to the stability and translocation of HIF-α to the nucleus, where it heterodimerizes with HIF-β [Id., citing Semenza, GL. Oncogene (2010) 29: 625-634]. The HIF-α/β dimer binds with the transcriptional coactivator p300/CBP and hypoxia response element to induce the expression of the HIF target gene located in the promoter region [Id., citing Majmundar, A J, et al. Mol. Cell (2010) 40: 294-309; Semenza, GL. Annu Rev. Pathol. (2014) 9: 47-71]. The development of an abnormal vasculature and a hypoxic microenvironment promotes abnormal angiogenesis, desmoplasia (meaning the formation of fibrous connective tissue by proliferation of fibroblasts), and inflammation, all of which contribute to tumor progression and therapeutic resistance [Id., citing Jain, RK. Cancer Cell (2014) 26: 605-622; Whatcott, C J et al. Cancer J. (2015) 21: 299-306].

In a hypoxic environment, activated HIF-1α increases the activity of Snail and Twist, two transcription factors that reduce E-cadherin expression and promote EMT. While EMT-related signaling is not required for the metastatic process, it promotes invasion, aging, cancer stem cell-like phenotype, and resistance to chemotherapy [Id., citing Thiery, J P, et al. Cell (2009) 139: 871-890]. HIF-1α can also intervene in the expression of enzymes that polymerize and regulate the alignment of collagen fibers and activity of integrins to promote cancer migration [Id., citing Semenza, GL. Annu Rev. Pathol. (2014) 9: 47-71]. Leaky and compressed blood and lymphatic vessels mediated by HIFs, such as angiopoietin-2, vascular endothelial growth factor (VEGF), and angiopoietin-like 4, facilitate the passage of metastatic cancer cells through the vessel wall [Id., citing Pastorek, J. and Pastorekova, S. Semin. Cancer Biol. (2015) 31: 52-64].

Glycolysis. The anoxic microenvironment is beneficial for glycolysis and lactic acid production by key enzymes of glycolysis and lactate dehydrogenase A (LDH-A); the excess production of lactic acid results in an acidic pH. Moreover, HIF can reversely convert carbon dioxide and water produced by the activation of carbonic anhydrase IX or XII into HCO₃⁻, which diffuses out of the cell membrane, resulting in excess HCO₃⁻ in the TME and a decrease in pH [Id., citing Harris, A L. Nat. Rev. Cancer (2002) 2: 38-47]. A large number of studies have concluded that the decreased intracellular pH of endosomes and lysosomes in tumor cells may assist in metastasis by activating proteases [Id., citing Nelson, D A, et al. Genes Dev. (2004) 18: 2095-2107; Pilon-Thomas, S. et al. Cancer Res. (2016) 76: 1381-1390].

Reactive oxygen species. The level of reactive oxygen species (ROS) has been shown to be increased in cancer cells exposed to hypoxia [Id., citing Zhu, X. and Zuo, L. Cell Death Dis. (2013) 4: e787]. The reduction in oxygen utilization decreases the passage of electrons through the mitochondrial complex by the electron transport chain (ETC), allowing electrons to leak from the ETC, thus leading to the overproduction of ROS [Id., citing Guzy, R D, et al. Cell Metab. (2005) 1: 401-408]. Moreover, the excessive production of ROS alters genomic stability and impairs DNA repair pathways [Id., citing Nita, M. and Grzybowski, A. Oxidative Med. Cell Longev. (2016) 2016: 3164734]. ROS can affect cell survival or apoptosis via oxidative stress, thus resulting in enhanced cytotoxicity and apoptosis [Id., citing Bridge, G., et al. Cancers (Basel) (2014) 6: 1597-1614]. At high concentrations (10-30 m), ROS can damage cellular biomolecules, such as proteins, DNA, and RNA, and cause mutations that promote cancer in normal cells or multidrug resistance (MDR) in cancer cells [Id., citing Syu, J P, et al. Oncotarget (2016) 7: 14659-14672]. However, most cancer cells still survive under internal oxidative stress, hence avoiding apoptosis and developing resistance to chemotherapy. Exposure to elevated levels of ROS can lead to cancer cell resistance by the activation of redox-sensitive transcription factors such as NF-κB, nuclear factor (erythroid-derived 2)-like factor 2 (Nrf2), c-Jun, and HIF-1α [Id., citing Shen, Y, et al. Exp. Cell Res. (2015) 334(2): 207-218]. Subsequently, the activation of these genes enhances the activation of the antioxidant system and promotes the expression of cell survival proteins. In addition, ROS facilitate the transition from apoptosis to autophagy in methotrexate-resistant choriocarcinoma jeg-3 cells, enabling the survival of cells to methotrexate [Id., citing Corzo, C A, et al. J. Exp. Med. (2010) 207: 2439-2453]. ROS can also stimulate the differentiation of cancer stem cells, thus promoting epithelial-mesenchymal transition (EMT) and inducing metabolic reprogramming involved in the resistance of cancer cells.

Epithelial-mesenchymal transition. EMT is a key process in the metastasis and colonization of cancer cells from the primary tumor to distant organs. HIF has a direct regulatory effect on EMT-related proteins, such as zinc finger E-box binding homeobox 1, Snail and Twist [Yang, M H, et al. Nat. Cell Biol. (2008) 10 (3): 295-305; Zhang, W., et al. PLoS One (2015) 10(6): e0129603; Xi, Y, et al. Mol. Cancer (2022) 21 (1): 145]. At the same time, HIF can also modulate microRNA (miRNA) to promote the cellular EMT process [Xi, Y, et al. Mol. Cancer (2022) 21 (1): 145; Li, H., et al. Gastroentrology (2017) 153(20: 505-520; Xu, Q., et al. Mol. Cancer (2017) 16 (1): 103; Xing, S., et al. Mol. Cancer (2021) 20 (1): 9].

Immunosuppression. Hypoxic stress causes immunosuppression by controlling angiogenesis and favoring immune suppression and tumor resistance. Macrophages constitute a principal component of the immune infiltrate in solid tumors by differentiating into tumor-associated macrophages (TAMs), which have been found to be preferentially located in tumor hypoxic areas [Jing, X., et al. Molecular Cancer (2019) 18: 157, citing Mantovani, A. et al. Trends Immunol. (2002) 23: 549-555]. Tumor-derived cytokines are able to convert TAMs into polarized type 2, or M2, macrophages with more immunosuppressive activities, resulting in tumor progression. Myeloid-derived suppressor cells (MDSCs) can directly promote immune tolerance [Id., citing Noman, M Z, et al., J. Exp. Med. (2014) 211: 781-790]. In hypoxic zones, HIF-1 directly regulates the function and differentiation of MDSCs, and such tumor-derived MDSCs are more immunosuppressive compared with splenic MDSCs. The upregulation of the expression of programmed death-ligand 1 (PD-L1) under hypoxia has been shown [Id., citing Barsoum, I B, et al. Cancer Res. (2014) 74: 665-674]. Further evidence supports that HIF-1 is a major regulator of PD-L1 mRNA and protein expression. HIF-1 regulates the expression of PD-L1 by binding directly to a hypoxia response element in the PD-L1 proximal promoter [Id., citing Noman, M Z, et al. J. Exp. Med. (2014) 211: 781-790]. The originally elevated immunosuppressive function of tumor-derived MDSCs under hypoxia was found to be abrogated following PD-L1 blockade. Along with PD-L1 blockade, the hypoxia-mediated upregulation of IL-6 and IL-10 in MDSCs was significantly attenuated [Id., citing Saggar, J K, et al. Front. Oncol. (2013) 3: 154].

At present, immunotherapeutic strategies triggering antitumor immunity are not effective because of diverse mechanisms of tumor escape from immunosurveillance. The antibody blockade of the T-cell immune checkpoint receptors PD-1 and CTLA-4 was poor in some tumors because T cells were sparse or absent in the TME; the hypoxia-driven modulation of T-cell exclusion and apoptosis help maintain this state. Although T cells can enter hypoxic tumors, the hypoxia-mediated acidification of the extracellular milieu blocks the capacity of T cells to expand or perform cytotoxic effector functions.

Hypoxia leads to a decreased pH in the TME. Since some chemotherapeutic drugs currently used in clinical practice are pH dependent in terms of their intracellular targets, changes in the intracellular pH gradient result in decreased drug accumulation in tumor cells, thereby greatly reducing the efficacy of chemotherapeutic drugs and eventually leading to drug resistance.

Defective apoptosis Anticancer treatments act in part by inducing apoptosis [Id., citing Maddika, S., et al. Drug Resist. Updat. (2007) 10: 13-29; Enari, M. et al. Nature (1998) 391: 43-50]. Tumor cells always alter their metabolism to ensure survival and evade host immune attack to proliferate. Under hypoxic conditions, nonadaptive cancer cells undergo apoptosis via HIF-1- and P53-dependent mechanisms.

### Tirapazamine

Tirapazamine (3-amino-1,2,4-benotriazine-1,4-di-N-oxide, or SR 4233), the structural formula of which is shown below,

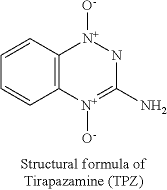

is a bioreductive agent that has significantly higher cytotoxicity under hypoxic conditions compared to under a normal oxygenated environment [Brown, J M. Br. J. Cancer (1993) 67: 1163-1170]. The cytotoxic effect of Tirapazamine is mediated by formation of hydroxyl free radicals under a hypoxia environment [Abi-Jaoudeh, N. et al. J. Hepatocellula Carcinoma (2021) 8; 421-434], free radical-induced DNA strand breaks and organelle/cell membrane damage. Toxicology studies in mice determined the dose at which 10% of individuals in a population will die ((LD₁₀) of TPZ as 294 mg/kg by intravenous administration, and the toxicity increases steeply above LD₁₀ with the dose at which 50% of individuals in a population will die (LD₅₀) at 303 mg/kg. Phase I human clinical studies showed that tirapazamine as a single agent administered intravenously every three weeks has a Maximally Tolerated Dose (MTD) of 390 mg/m². Pharmacokinetic analysis showed that the mean terminal half-life was very short at approximately 40 min. [Senan, S., et al. Clin. Cancer Res. (1997) 3 (1): 31-38].
Pharmacokinetic and Toxicology Evaluation with i.v. Tirapazamine as a Single Agent

Dose escalation studies of tirapazamine as a single agent administered by intravenous injection were conducted in a Phase 1 study in patients with histologically proven cancer that were refractory to conventional chemotherapeutic agents, the results of which were published in the scientific literature [Senan, S., et al. Clin. Cancer Res. (1997) 3 (1): 31-38]. None of the patients received chemotherapy, radiotherapy, or immunotherapy in the 3 weeks before tirapazamine administration (6 weeks in the case of nitrosoureas and mitomycin C). In this study, tirapazamine was administered via i.v. injection once every three weeks. The goals of this study were to establish the toxicity profile and the MTD, to study the plasma pharmacokinetics of tirapazamine and its metabolites, and in turn to correlate this with toxicity. A total of 28 patients were given 50 courses of tirapazamine at doses ranging from 36-450 mg/m² according to a modified Fibonacci dose escalation scheme. The starting dose was based on the results of toxicology studies performed in mice, rats, and dogs. Tirapazamine was rapidly cleared from plasma with a mean clearance (±SD) of 624.2±157 mL/min and mean Vdₛₛ of 39±12.5 liters. Plasma tirapazamine levels decreased with a mean terminal half-life of 46.6±9.53 min. In some individuals, a short initial distribution phase and/or a prolonged terminal phase was observed that could not be characterized accurately. The inter-patient variability in tirapazamine AUC was relatively limited at all dose levels. The mean AUC_{(0-inf)} increased with dose in a greater than dose-proportional manner (P<0.001) with a 12.5-fold increase in dose producing an estimated 19-fold (CI, 14.9-24.3) increase in AUC_{(0-inf)}. Tirapazamine Cₘₐₓ values significantly increased with dose (P<0.001) although in a less than dose-proportional manner, due to progressive increase in infusion time implemented during dose escalation. The t_{1/2} of tirapazamine increased significantly with dose (P=0.014); this was accompanied by a slight but significant decrease in clearance (P=0.016). There was no significant dose effect on Vdₛₛ (P=0.282).

Although the mean AUC values were in the estimated range required for therapeutic effect in murine studies, no tumor responses were seen. The dose-limiting toxicities observed were reversible deafness and tinnitus. Ototoxicity was observed in 1 of 6 patients treated at the 330 mg/m² dose, 1 of 4 patients treated at 390 mg/m² dose, and 3 of 3 patients treated at 450 mg/m² dose. Patients who displayed ototoxicity generally showed greater plasma AUC values for tirapazamine and its metabolites. Muscle cramps, nausea, and vomiting were also observed. Ototoxicity was not observed when the AUC of tirapazamine was equal to or less than 1252 μg/mL×min (330 mg/m² dose). Therefore the 330 mg/m² dose via i.v. was selected as an appropriate level for combination chemotherapy studies. The maximum tolerated dose (MTD) was found to be 390 mg/m².

Ototoxicity symptoms commenced in the first 48 h after the start of drug infusion, and the severity varied with tirapazamine dose. Subjective hearing loss was most prominent in patients treated at the 450 mg/m² dose level. Except for a patient who was treated at 450 mg/m², the ototoxicity symptoms resolved completely in all patients. No evidence of cumulative ototoxicity was observed.

Muscle cramps occur in patients at all dose levels except the lowest one at 36 mg/m². The onset of cramps was generally between 2.4-24 hours after the start of infusion, but it was delayed up to 5 days in 1 patient. Typically, cramps began on waking up in the morning, affected mainly the lower limbs, and were relieved by weight-bearing or stretching the affected muscle. The episodes were generally mild and transient and did not increase in severity with dose or after retreatment at the same dose. The duration of cramps varied from 1-14 days, and the cramps persisted longer in the 3 patients treated at 120 mg/m² (14 days in all 3 patients) than in those treated at 450 mg/m² (0, 1, and 1 days respectively). Creatine phosphokinase (CPK) enzyme levels were not elevated in those patients after the onset of cramps. No electrolyte abnormalities were observed in patients with cramps and no patient developed signs of peripheral neuropathy. Administration of diazepam did not influence the incidence of muscle cramps.

Initial clinical development of tirapazamine was conducted in combination with conventional chemotherapy or chemoradiation in the 1990's. However, the developmental path was terminated after failure of three phase 3 randomized studies [Williamson S K, et al. J. Clinical Oncol. (2005) 23 (36): 9097-9104; Rischin D, et al. J. Clinical Oncol. (2010) 28 (18): 2989-2995; (DiSilvestro P A, et al. J. Clinical Oncol. (2014) 32 (5): 458-464).

The results of another randomized phase III trial in NSCLC patients were published [Williamson, S K, et al. J. Clinical Oncol. (2005) 23 (36): 9097-9104]. The goal of this phase III clinical trial was to determine whether the addition of tirapazamine to paclitaxel and carboplatin offered a survival advantage when used in the treatment of patients with advanced NSCLC. The trial enrolled 396 patients with histologically or cytologically confirmed NSCLC (categorized as squamous cell, large cell, adenocarcinoma, or NSCLC not otherwise specified) with stage IV (no brain metastases) or selected stage IIIB disease (pleural effusion or multiple ipsilateral lung nodules) by the International Staging System for lung cancer. Three hundred and sixty-seven (367) eligible patients were randomly assigned to either arm 1 (n=181), which consisted of treatment every 21 days with paclitaxel 225 mg/m² over 3 hours, carboplatin (AUC=6), and tirapazamine 260 mg/m² in cycle 1 (which was escalated, if tolerable, to 330 mg/m² in cycle 2), or arm 2 (n=186), which consisted of paclitaxel and carboplatin as in arm 1 with no tirapazamine. Patient characteristics were similar between the two arms. There were no statistically significant differences in response rates, progression-free survival, or overall survival. However, patients in arm 1 had significantly (P<0.05) more abdominal cramps, fatigue, transient hearing loss, febrile neutropenia, hypotension, myalgias, and skin rash and were removed from treatment more often as a result of toxicity than were patients in arm 2 (26% vs 13%, respectively; P=0.003). Twenty patients on the tirapazamine arm (arm 1) developed ≥grade 3 febrile neutropenia compared with 6 patients on arm 2 (P=0.004). Grade 3 and grade 4 peripheral neuropathy and other grade 3 and 4 nonhematologic toxicities were similar between the two arms. More than 40% of patients in arm 1 did not have the tirapazamine dose escalated as planned, primarily because of toxicity. The trial was closed early after an interim analysis demonstrated that the projected 37.5% improvement in survival (8 vs 11 months median survival) in arm 1 was unachievable (P=0.003). The authors concluded that the addition of tirapazamine to paclitaxel and carboplatin does not result in improved survival in advanced NSCLC compared with paclitaxel and carboplatin alone but substantially increases toxicity.

Based on the promising efficacy seen in phase II trials in combination with chemoradiation in head and neck cancer, a large open-label randomized phase III trial was initiated by the Trans-Tasman Radiation Oncology Group. [Rischin, D., et al. J. Clinical Oncol. (2010) 28 (18): 2989-2995]. The goal of this study was to evaluate radiation and cisplatin with or without tirapazamine with the primary end point being overall survival (OS). Patients with previously untreated stage III or IV (excluding T1-2N1 and M1) squamous cell carcinoma (SCC) of the oral cavity, oropharynx, hypopharynx, or larynx were randomly assigned to receive definitive radiotherapy (70 Gy in 7 weeks) concurrently with either cisplatin (100 mg/m²) on Day 1 of weeks 1, 4, and 7 or cisplatin (75 mg/m²) plus tirapazamine (290 mg/m²/d) on Day 1 of weeks 1, 4, and 7 and tirapazamine alone (160 mg/m²/d) on Days 1, 3, and 5 of weeks 2 and 3 (tirapazamine/cisplatin). Eight hundred sixty-one (861) patients were accrued from 89 sites in 16 countries. As anticipated, muscle cramps, diarrhea, and skin rash were more frequent in the tirapazamine arm, however no difference in the incidence of death was observed or febrile neutropenia between the two arms. In an intent-to-treat analysis, the 2-year OS rates were 65.7% for CIS and 66.2% for tirapazamine/cisplatin (95% CI, −5.9% to 6.9%). There were no significant differences in failure-free survival, time to locoregional failure, or quality of life as measured by Functional Assessment of Cancer Therapy-Head and Neck. Therefore, in this definitive large scale phase III study, there was no evidence that the addition of tirapazamine to chemoradiotherapy in patients with advanced head and neck cancer not selected for the presence of hypoxia improves OS.

A phase III randomized clinical study, an intergroup trial by the Gynecologic Oncology Group (GOG) and National Cancer Institute of Canada Clinical Trials Group, was designed to test the effectiveness and safety of adding the hypoxic cell sensitizer tirapazamine (TPZ) to standard cisplatin (CIS) chemoradiotherapy in locally advanced cervix cancer [DiSilvestro, P A, et al. J. Clinical Oncol. (2014) 32 (5) 458-464]. 387 patients were randomized into two arms and received cisplatin-based chemoradiation (CIS/RT) with or without TPZ over a 36-month period of time. Due to the lack of TPZ supply, the study did not reach its original target accrual goal. At median follow-up of 28.3 months, progression-free survival (PFS) and OS were similar in both arms. Three-year PFS for the TPZ/CIS/RT and CIS/RT arms were 63.0% and 64.4%, respectively (log-rank P=0.7869). Three-year OS for the TPZ/CIS/RT and CIS/RT arms were 70.5% and 70.6%, respectively (log-rank P=0.8333). A scheduled interim safety analysis led to a reduction in the starting dose for the TPZ/CIS arm, with resulting tolerance in both treatment arms.

The study concluded that TPZ/CIS chemoradiotherapy was not superior to CIS chemoradiotherapy in either PFS or OS, although a definitive conclusion was limited by an inadequate number of events (progression or death). TPZ/CIS chemoradiotherapy was tolerable at a modified starting dose. The reported safety profile of the TPZ/CIS was in line with the prior reports, in which TPZ was combined with chemoradiation in squamous carcinoma of Head and Neck [Rischin, D., et al. J. Clinical Oncol. (2010) 28 (18): 2989-2995]. This is the third randomized study that failed to demonstrate that addition of TPZ to chemotherapy or chemoradiation can significantly improve therapeutic efficacy of the standard care therapy.

Interest in tirapazamine has been rejuvenated by its combination with hepatic artery ligation [Lin W H, et al. Proc. Natl Acad. Sci. USA (2016) 113 (42): 11937-11942). The combination of tirapazamine and transarterial embolization (TAE) is a rational approach to use with tirapazamine since the agent is most cytotoxic under conditions of hypoxia. This hypothesis was evaluated in a non-GLP study using a murine animal model and Hepatic artery ligation (HAL) in lieu of TAE in these animals. Hepatitis B Virus X (HBx) transgenic mice, which spontaneously develop hepatocellular carcinoma (HCC) after 18 months of age due to the expression of HBx, have the advantage of low background tumor necrosis, shared mechanism of tumorigenesis as the HBV-related HCC in humans, and similar underlying hepatic dysfunction as observed in HCC patients.

After determination of a potentially tolerable dose for tirapazamine in combination with transient left HAL in wild-type mice, the effect in the precancerous liver of HBx transgenic mice (age: 13-15 months) that do not have tumors was evaluated. The dose-escalation analysis was divided into two groups. One group of mice was sacrificed 1 day post treatment with either saline (n=2) or i.v. tirapazamine at doses of 6 mg/kg and 3 mg/kg (n=2 for each group). The other group was sacrificed 7 days post-treatment with saline (n=2) or tirapazamine at doses of 6 mg/kg and 3 mg/kg (n=3 for each group). All mice received transient HAL of the left liver lobe.

The pO2 and blood flow analysis using an oxyLite/oxyFlo combinational sensory probe showed that no hypoxia was established in the precancerous liver of HBx transgenic mice by transient left HAL. After 1 and 7 days, all mice receiving left HAL and saline or 3 mg/kg or 6 mg/kg tirapazamine survived. There were similar body weight change and normal range of total bilirubin levels in serum after transient left HAL combined with tirapazamine, but serum alanine transaminase (ALT) level was elevated on Day 1 in all tirapazamine-treated groups in a dose-dependent manner. Histopathology examination of the left lobe of the liver showed 1% necrosis in mice treated with 3 mg/kg and 6 mg/kg rirapazamine, which was consistent with the elevation of ALT on Day 1. However no histopathological changes were seen on Day 7, suggesting that the liver injury on Day 1 was transient, and fully recovered on Day 7. This result showed that there were no major differences between the normal liver of C57BL/6 mice and the precancerous liver of HBx transgenic mice with regard to tirapazamine toxicity.

Based on this study, the potentially safe doses of tirapazamine combined with transient left HAL in this model were shown to be 6 mg/kg or lower by i.v. infusion. However, the HCC bearing HBx transgenic mice are physically ill and it is possible that liver function is more compromised than in the precancerous liver. Therefore, a lower dose of 3 mg/kg of tirapazamine i.v. was chosen for HCC treatment and efficacy analysis when combined with left HAL.

Efficacy Analysis of Tirapazamine Versus Doxorubicin Combined with HAL in HBx Transgenic Mice

With the establishment of an appropriate dose of tirapazamine in the HBx transgenic mouse model combined with transient HAL, the efficacy of tirapazamine was investigated and compared with a commonly used chemotherapy agent, doxorubicin, in transarterial chemoembolization (TACE). A small group was first tested to compare the effects of saline (n=1), doxorubicin (10 mg/kg, n=1) and tirapazamine (3 mg/kg, n=1) to treat HBx transgenic mice that had palpable HCC, along with transient left HAL. Mice were sacrificed 1 day after treatment with tirapazamine and transient left HAL as previously described. The ALT level had a much higher elevation in tirapazamine treated mice than in doxorubicin-treated mice. The histopathological examination at Day 1 post-treatment showed that tirapazamine induced more than 99% necrosis in the HCC within the territory of HAL, in contrast to about 5% necrosis in doxorubicin-treated HCC. This indicated that tirapazamine was much more effective than doxorubicin when combined with HAL.

Next the same study was conducted to examine the histopathology changes at 7 days post treatment. The number of mice used for each group were saline (n=2), doxorubicin (10 mg/kg, n=2) and tirapazamine (3 mg/kg, n=3). Tumor blood flow was monitored by the oxyFlo sensor and showed that blood flow dropped to 30% by HAL for HCC treated with either tirapazamine or doxorubicin, which was sufficient to induce tumor hypoxia.

Analysis of mouse bodyweight showed no statistically significant change, but the average weight in doxorubicin-treated mice appeared slightly lower (data not shown). The levels of total bilirubin in serum were all within the normal range among the three groups throughout the 7 days (data not shown). The serum ALT level reached a peak on Day 1 in the group treated with tirapazamine and transient left HAL, which was similar to previous observations (data not shown). Subsequently, ALT levels decreased and returned to normal around Day 3. Doxorubicin and transient HAL also induced ALT elevation on Day 1 but less than tirapazamine and left HAL, and both recovered to normal levels by Day 2. During dissection on Day 7, it was evident that tumor necrosis occurred in HCC treated with tirapazamine and transient HAL by its pale color, but not in HCC from mice treated with saline or doxorubicin (data not shown). Histopathologic analysis by H&E staining confirmed that HCCs in the territory of the left HAL had 90-99% necrosis after the combination treatment of tirapazamine and left HAL. Little to no pathological changes were detected in HCCs treated with saline or doxorubicin combined with left HAL (data not shown). Since these mice frequently had multiple HCC, there were tumor nodules present in the right lobe that served as internal controls. Tumor necrosis did not occur in any of the right lobes in any animal. In the tumor-free part of the left lobe of the liver, there was no evidence of necrosis seen, indicating that the combination of tirapazamine and left HAL did not result in any significant liver injury in the same lobe by Day 7. Although there may have been transient damage on Day 1 as suggested by elevation of ALT, it was fully recovered on Day 7.

To examine any residual viable tumor that could be present, the whole tumors from the group treated with tirapazamine and left HAL were dissected and examined extensively. Particular attention was focused in the peripheral region since it was possible that HCC could have oxygen provided by diffusion and leave some viable tumor in the peripheral area. The three mice treated with a combination of tirapazamine and left HAL induced tumor necrosis at 90%, 99% and 99%, respectively (data not shown). Tumor necrosis was also observed in the peripheral tumor located next to the normal liver or outer capsules. These results indicated that tirapazamine is an effective cytotoxic agent when combined with hypoxic conditions induced by HAL.

The success of this animal model study lead to a new strategy involving combination of tirapazamine with trans-arterial embolization (TAE), using the TAE to induce profound tumor hypoxia to activate tirapazamine. Two phase I studies using this strategy showed promising results with a Complete Response (CR) rate at 50-60% [Abi-Jaoudeh N, et al. J. Hepatocellular Carcinoma (2021) 8: 421-434; Liu C H, et al. J. Vascular & Interventional Radiology (2022) 33 (8): 926-933).

### LT-001 TATE Monotherapy Study

A phase 1 dose-escalating study was completed in patients with unresectable intermediate stage HCC and published by Abi-Jaoudeh, et al. [J. Hepatocellular Carcinoma (2021) 8: 421-434]. The maximal TPZ dose was 10 mg/m2 IV. and 20 mg/m2 I.A. Among the 27 enrolled HCC patients, 25 were evaluable for efficacy analysis. Two patients were lost to follow-up.

### Safety Evaluation

All the enrolled patients tolerated the study treatment well. There was no dose-limiting toxicity (DLT) in all cohorts, and MTD of tirapazamine in combination with TAE was not established. Dose escalation was terminated due to a plateaued efficacy at 20 mg/m² administered intra-arterially (IA). This dose is far lower than those used in prior phase 3 trials at 260-330 mg/m² i.v. and MTD of 390 mg/m² i.v., although via a different route of administration. Subsequent patients in the expansion cohort were treated with a flat dose of 35 mg, selected based on rounded up from the dose of 20 mg/m². No patient required any dose modification from the planned tirapazamine dose in all subsequent treatments. The treatment was done “on demand”. Once CR by mRECIST was achieved, patients were observed during the regular follow-up and treated only if they were documented to have disease progression or recurrence. Treatment can continue as long as patients are not refractory toward TATE or until patients have extra-hepatic progression or death.

Treatment emergent AEs with grade 3 or higher with an incidence above 5% included hypertension (25.9%), AST increased (14.8%), and ALT increased (11.1%). The treatment-related AEs with an incidence above 5% included hypertension (11.1%), AST increased (7.4%), bradycardia (7.4%), ALT increased (7.4%), and fatigue (7.4%).

### Efficacy Evaluation

Among the 27 enrolled treatment-naïve patients, all received cycle 1, 14 patients (52%) received cycle 2, 6 (22%) received cycle 3, and 2 (7%) received cycle 4. The average number of treatments per patient was 1.8 treatments over a median duration on study of 359 days (range 9-723). Due to the nature of the single arm phase I study, the efficacy was assessed by the complete response (CR) rate, overall response rate (ORR), and duration of CR or response (CR+PR). The response was assessed by MRI scans using both mRECIST and RECIST criteria. MRI scans are more appropriate than contrast-enhanced CT scans to evaluate HCC response after Lipiodol-based TACE due to accumulation of Lipiodol in the embolized territory that interferes with the assessment of the tumor viability using contrast CT scans. CR is a high bar with even more predictive to a long OS. The duration of CR or sustained CR by mRECIST was associated with a low likelihood of recurrence and prolongation of OSⁱ. Achieving durable CR also spares patients from further treatment until progression and may bring additional benefits such as better quality of life and reduced healthcare costs.

The average number of treatments per patient was 2.2 over a median follow-up of 303 days (range=23-856 as of Mar. 5, 2019). Twenty-five patients were evaluable for treatment response, and the best response was shown by Waterfall plots (data not shown). Among the 25 patients, 15 (60.0%, 95% CI: 38.7, 78.9) achieved CR in target lesions, and 21 (84.0%, 95% CI: 63.9, 95.5) achieved CR+PR based on mRECIST (data not shown). If counted by tumor lesions, there were 46 target lesions in these 25 patients. The CR rate by lesions is 30/46 or 65.2%, and the ORR (CR+PR) is 36/46 or 78.3% (data not shown).

The duration of CR or CR+PR was plotted in Kaplan Meier curve plots (data not shown). Both mRECIST and RECIST criteria were used for analysis to show the difference. Assessment by the mRECIST criteria, which measure the contrast-enhanced viable tumors, exhibited an early response at the first scheduled MRI scans, whereas assessment by the RECIST criteria showed a slower response since it takes time for necrotic tumor to be absorbed. The median duration of target lesion response was not reached (95% CI 103, NR) for 15 patients who achieved CR by mRECIST, and not reached (95% CI 197, NR) for the 21 patients who achieved CR or PR (data not shown).

Only 2/15 (13.3%) of the CR patients and 6/21 (28.6%) of CR+PR patients developed local recurrence in the embolized territory. The long duration of CR with a low incidence of local recurrence supports the theory that TATE treatment is capable of eradicating cancer stem cells.

The LT-002 study (Liu, C H, et al. J. Vascular and Interventional Radiology (2022): 33 (8): 926-933) enrolled Asian HCC patients who were in intermediate stage with the largest tumor no bigger than 10 cm, but with no limit in the number of tumor lesions except that if the number was over 5, they should be in the same lobe. All other eligibility was same as that of LT-001 study. The expansion cohort of the trial was divided into two groups, one without any prior embolization, and the other with prior embolization. The study population was therefore more advanced compared with that of LT-001 study [Abi-Jaoudeh, et al. J. Hepatocellular Carcinoma (2021) 8: 421-434]. The analysis results are described below.

The last visit of the last patient occurred in March 2020 and the database was locked in June 2022. In the analysis based on the final data, there were 17 patients enrolled and treated in this trial and treated with 5, 10 and 20 mg/m² intraarterially (I.A.) in a 3+3 design, followed by an expansion cohort using a fixed dose 35 mg I.A., which is equivalent to 20 mg/m² cohort. By the time of analysis on Jul. 17, 2021, the median range of follow-up was 5.7 months (range, 1.2-19.0 months). The median time on study is 170 days (95% CI, 52, NR) with a range from 36 to 569 days. Among the 17 patients, 8 (47.1%) patients achieved CR as the best target tumor lesion response (95% CI 23.0, 72.2), 11 (64.7%) achieved CR or partial response (PR), objective response rate (ORR) (95% CI, 38.3, 85.8) (data not shown). The best ORR is identical to the number of best target lesion response. Compared with those with or without prior TACE, the CR and ORR rates did not show any difference.

The duration of response has a range from 106 to 528 days, with the median not reached yet (data not shown). For the 8 patients who achieved CR, 5 of them still remain CR up to the date of analysis. For the 11 patients who achieved CR or PR, 8 remained in response, with median duration of response not reached.

The median Time to Progression by the mRECIST criteria (data not shown) was not reached (95% CI, 154, “NR”)

In all the clinical trials conducted to date, tirapazamine was formulated in a citrate acidic buffer with a pH of 4, in which the active ingredient tirapazamine was much more stable than in a basic condition. However, this acidic buffer formulation has a caveat of inducing significant pain during parenteral administration, and even worse during intra-arterial injection. When tirapazamine was combined with TAE and administered through a catheter placed in the hepatic artery, severe pain mandated general anesthesia during the procedure [Abi-Jaoudeh N, et al. J. Hepatocellular Carcinoma (2021) 8: 421-434; Liu, C H, et al. J. Vascular & Interventional Radiol. (2022) 33 (8): 926-933]. Another issue is that tirapazamine exhibits limited water solubility (less than 1 mg/mL) and it also takes time for tirapazamine to be dissolved into an aqueous solution.

In clinical usage, the volume of the tirapazamine drug product in the existing formulation and its excipient are proportionally increased with the dose increase due to the limitation of drug water solubility. The highly acidic citrate buffer at pH-4 poses a potential toxicity in causing metabolic acidosis from the buffer when the volume of the formulated drug product is large. In a toxicology study with mini-pigs, when a high dose of tirapazamine up to 120 mg/m² or higher was given to the mini-pigs, even though the dose of tirapazamine was still far below the MTD of human (390 mg/m²) and was supposed to be a non-toxic dose, the min-pigs died from severe acidosis within an hour after injection of the clinical batch of tirapazamine due to the toxicity of the pH 4 buffer. Therefore, there is an urgent need to develop a formulation that avoids these risks and toxicities.

The present disclosure provides formulations of tirapazamine with improved water solubility and without the strong acid condition of past formulations.

### SUMMARY OF THE INVENTION

According to one aspect, the present disclosure provides a cyclodextrin inclusion complex comprising a β-cyclodextrin host molecule substituted by hydroxypropyl groups (hydroxypropyl-β-cyclodextrin, or HPβCD) or by sulfopropylether groups (sulfobutylether-β-cyclodextrin or SBEβCD) and comprising a cavity containing tirapazamine as a guest, wherein (i) the tirapazamine guest is at least partially included into the cavity of the β-cyclodextrin host molecule; wherein the extent of inclusion ranges from about 1% to about 50%, inclusive; and (ii) a molar ratio of the cyclodextrin host to the tirapazamine guest ranges from about 14:1 to about 2:1, inclusive.

According to some embodiments of the cyclodextrin inclusion complex, the molar ratio of the β-cyclodextrin host to the tirapazamine guest in the complex is about 2:1; and a 0.7 mg/ml solution of tirapazamine complexed in at least a 1% solution of the substituted β-cyclodextrin is water soluble. According to some embodiments of the cyclodextrin inclusion complex, pH of the 0.7 mg/mL solution of tirapazamine complexed to the β-cyclodextrin ranges from about pH 5.3 to about pH 6.4, inclusive.

According to some embodiments of the cyclodextrin inclusion complex, the dissolved complex is stable for at least 24 hr when stored at 20°−25° C. (room temperature) or at 5° C.

According to some embodiments of the cyclodextrin inclusion complex, the β-cyclodextrin host molecule is substituted by hydroxypropyl groups (hydroxypropyl-β-cyclodextrin, or HPβCD). According to some embodiments of the cyclodextrin inclusion complex, solubility of the complexed TPZ in at least a 1% solution of the HPβCD host at room temperature ranges from about 0.7 mg/mL to 2.55 mg/mL, inclusive, at a pH range of about 5.8 to 6.2, inclusive.

According to some embodiments of the cyclodextrin inclusion complex, solubility of the complexed TPZ in at least the 1% solution of the HPβCD host at room temperature at a molar ratio of the β-cyclodextrin host to the tirapazamine guest of 2.0 is about 0.7-1 mg/mL at pH of 6.

According to another aspect, the present disclosure provides a pharmaceutical composition comprising a cyclodextrin inclusion complex comprising a β-cyclodextrin host molecule substituted by hydroxypropyl groups (hydroxypropyl-β-cyclodextrin, or HPβCD) or by sulfopropylether groups (sulfobutylether-β-cyclodextrin or SBEβCD) and comprising a cavity containing tirapazamine as a guest, wherein: the carrier is an aqueous carrier; the tirapazamine guest is at least partially included into the cavity of the β-cyclodextrin host molecule; the extent of inclusion ranges from about 1% to about 50%, inclusive; and a molar ratio of the cyclodextrin host to the tirapazamine guest ranges from about 14:1 to about 2:1, inclusive.

According to some embodiments of the pharmaceutical composition, the molar ratio of the cyclodextrin host to the tirapazamine guest is about 2:1; the β-cyclodextrin host molecule is substituted by hydroxypropyl groups (hydroxypropyl-β-cyclodextrin, or HPβCD); and about a 0.7-1 mg/mL solution of the complexed tirapazamine guest in at least a 1% solution of the substituted β-cyclodextrin host is water soluble.

According to some embodiments of the pharmaceutical composition, pH of the solution comprising the tirapazamine guest complexed with the β-cyclodextrin host ranges from about pH 5.3 to about pH 6.4, inclusive.

According to some embodiments of the pharmaceutical composition, the pharmaceutical composition comprising the complexed tirapazamine comprises improved stability at room temperature compared to the stability of non-complexed tirapazamine alone.

According to some embodiments of the pharmaceutical composition, the aqueous carrier is water, normal saline, Ringer's solution or a dextrose solution.

According to some embodiments of the pharmaceutical composition, the pharmaceutical composition comprising the β-cyclodextrin-complexed tirapazamine is formulated for administration intra-arterially or by intravenous infusion.

According to some embodiments of the pharmaceutical composition, the pharmaceutical composition comprising the β-cyclodextrin-complexed tirapazamine comprises reduced toxicity of injection-related pain when compared to the toxicity of the non-complexed tirapazamine alone.

According to another aspect, the present disclosure provides a method of treating a liver tumor comprising (a) targeting the liver tumor by administering a pharmaceutical composition comprising a cyclodextrin inclusion complex comprising a β-cyclodextrin host molecule substituted by hydroxypropyl groups (hydroxypropyl-β-cyclodextrin, or HPβCD) comprising a cavity containing tirapazamine as a guest, wherein the carrier is an aqueous carrier; pH of a 0.7-1 mg/mL aqueous solution of the complexed tirapazamine guest ranges from pH 5.3 to 6.4, inclusive; the tirapazamine guest is at least partially included into the cavity of the β-cyclodextrin host molecule, wherein the extent of inclusion ranges from about 1% to about 50%, inclusive; and a molar ratio of the cyclodextrin host to the tirapazamine guest ranges from about 14:1 to about 2:1, inclusive; (b) transiently ligating the hepatic artery of the subject so that the cyclodextrin inclusion complex comprising tirapazamine is transiently retained within liver tissue comprising the liver tumor; and (c) producing targeted necrosis within the liver tumor and not viable liver tissue.

According to some embodiments of the method, the pharmaceutical composition comprising the cyclodextrin inclusion complex of tirapazamine comprises reduced toxicity of injection-related pain when compared to noncomplexed tirapazamine alone.

According to some embodiments of the method, the transient ligation of the hepatic artery is for a time period of at least about 40 minutes.

According to some embodiments of the method, the administering is intra-arterially or by intravenous infusion.

According to some embodiments of the method, the molar ratio of the cyclodextrin host to the tirapazamine guest is about 2:1; the β-cyclodextrin host molecule is substituted by hydroxypropyl groups (hydroxypropyl-β-cyclodextrin, or HPβCD); and a 0.7-1 mg/mL solution of the tirapazamine guest complexed with at least a 1% solution of the substituted cyclodextrin host is water soluble.

According to some embodiments of the method, the aqueous carrier is water, normal saline, Ringer's solution or a dextrose solution.

According to some embodiments of the method, the liver tumor is a hepatocellular carcinoma.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the structures of the three native cyclodextrins. (Taken from Poulson, B G et al. Polysaccharides (2022) 3: 1-31).

FIG. 2 shows the appearance of the tirapazamine starting material used in the examples.

FIG. 3 shows the appearance of 1 mg/ML tirapazamine in 5% and in 2.5% hydroxypropyl beta cyclodextrin (HPβCD at t=0 and after 24 hours

FIG. 4 shows the appearance of tirapazamine in sulfobutylether-β-cyclodextrin (SBEβCD) after 14 hours at room temperature.

FIG. 5 shows the appearance of tirapazamine/benzyl alcohol (TPz/BA) and TPZ/nicotinamide suspended in water.

FIG. 6 shows the appearance of tirapazamine/arginine and TPZ/meglumine in sterile water for injection (WFI).

FIG. 7 is a plot of solubility (mg/ml) on the Y axis and molar ratio (HPβCD:TPZ) on the Y axis. It shows the correlation between TPZ solubility and the molar ratio of HPβCD:TPZ.

FIG. 8 is a hypothetical structure of the TPZ-HPβCD inclusion complex at 1 mg/ml

FIG. 9 is a plot of pH on the Y axis versus molar ratio (HPβCD:TPZ on the X axis. It shows a strong linear relationship between pH and the molar ratio of HPβCD/TPZ.

FIG. 10 shows the appearance of 0.6 mg/mL tirapazamine in three different vehicles. In FIG. 10A, vehicle is HPβCD at pH 6. In FIG. 10B, vehicle is citrate buffer 140 mM at pH 6. In FIG. 10C, vehicle is normal saline.

FIG. 11 shows Hematoxylin & Eosin (H & E) staining of representative formalin-fixed paraffin-embedded (FFPE) liver from Group 1-9 showing normal liver tissue (no evidence of necrosis).

FIG. 12 shows H & E staining of representative FFPE liver tumor tissue from Group 1-9 showing no evidence of necrosis.

FIG. 13 shows H & E staining of representative FFPE liver tumor tissue from Group 1-10 showing no evidence of necrosis.

FIG. 14 shows H & E staining of representative FFPE liver tissue from Group 1-11 showing no evidence of necrosis in either normal liver (left) or liver tumor tissue(right).

FIG. 15 shows H & E staining of representative FFPE liver tissue from Group 1-11 showing no evidence of necrosis in either normal liver or liver tumor tissue.

FIG. 16A shows H & E staining of a representative FFPE Group 2-1 liver tumor with extensive necrosis at a low power view (100×). FIG. 16B shows liver tumor at a high power magnification showing the liver tumor necrosis with inflammatory infiltrates.

FIG. 17 shows H & E staining of a representative FFPE Group 2-2 necrotic liver tumor.

FIG. 18 shows H & E staining of a representative FFPE Group 2-4 liver tumor showing an area of complete necrosis surrounded by viable tumor tissue.

FIG. 19 shows H & E staining of a representative FFPE Group 3-6 liver tumor showing an area of complete necrosis surrounded by viable tumor tissue.

FIG. 20 shows H & E staining of a representative FFPE Group 3-7 liver tumor showing an area of necrosis surrounded by viable tumor and normal liver tissue.

FIG. 21 shows H & E staining of a representative FFPE Group 3-7 liver tissue showing a necrotic tumor surrounded by viable tumor tissue.

FIG. 22 shows H & E staining of a representative FFPE Group 3-8 liver tumor showing an area of complete necrosis and an area of viable tumor tissue.

FIG. 23 shows pharmacokinetic (PK) analysis of tirapazamine compositions. Plasma concentrations of Tirapazamine were determined in collected PK samples by LC-MS after rats were injected with Tirapazamine in normal saline (NS) vs. in HPβCD (HBC) at 3.33 mg/kg or 7 mg/kg intra-arterially.

FIG. 24 shows PK analysis of SR4317, a metabolite of Tirapazamine. Plasma concentrations of SR4317 were determined in collected PK samples by LC-MS after rats were injected with Tirapazamine in normal saline (NS) vs. in HPβCD (HBC) at 3.33 mg/kg or 7 mg/kg intra-arterially.

FIG. 25 shows PK analysis of SR4330, another metabolite of Tirapazamine. Plasma concentrations of SR4330 were determined in collected PK samples by LC-MS after rats are injected with Tirapazamine in normal saline (NS) vs. in HPβCD (HBC) at 3.33 mg/kg or 7 mg/kg intra-arterially.

### DETAILED DESCRIPTION OF THE INVENTION

### Definitions

As used herein and in the appended claims, the singular forms “a” “an”, and “the” include plural reference unless the context clearly dictates otherwise. Thus, for example, reference to a “peptide” is a reference to one or more peptides and equivalents thereof known to those skilled in the art, and so forth.

As used herein, the term “about” means plus or minus 20% of the numerical value of the number with which it is being used. Therefore, for example, about 50% means in the range of 40%-60%, inclusive, i.e., 40%, 41%, 42%, 43%, 44%, 45%, 46%, 47%, 48%, 49%, or 50%.

The term “active” refers to the ingredient, component or constituent of the compositions of the described invention responsible for the intended therapeutic effect.

The term “adverse event” or “AE” as used herein refers to an unfavorable medical event that occurs in a subject who is given a therapeutic product but does not necessarily have a causal relationship with the treatment. It may be any adverse and unwanted signs (including an abnormal laboratory result), symptoms, or temporary illness associated with the use of the product, whether or not it is related to the product. The correlation between adverse events and test medications is affirmative, likely related, may be relevant, may be irrelevant, and certainly not relevant. The severity of adverse events according to the National Cancer Institute's Common Terminology Criteria for Adverse Events (CTCAE) Scale is shown in Table 1 below.

TABLE 1
Severity of Adverse Events

| CTCAE Scale | Description |
|---|---|
| Mild (Grade 1) | Asymptomatic or mild; or only seen clinically or |
| | diagnostically; or no treatment required |
| Moderate (Grade 2) | Requires minor, local or non-invasive treatment; |
| | age-appropriate instrumental activities of daily |
| | life are limited |
| Severe (Grade 3) | Serious or medically significant but not immediately |
| | life-threatening; resulting in hospitalization or |
| | prolonged hospital stay; disability; limited daily |
| | activities of the individual |
| Life Threatening | Life-threatening; urgent treatment required |
| (Grade 4) | |
| Death (Grade 5) | Death associated with AE |

The term “alanine transaminase” or “ALT” as used herein refers to an enzyme found inside liver cells.

The term “aspartate transferase” or “AST” refers to a liver enzyme that is released in the blood when the liver is damaged.

The term “angiogenesis” as used herein refers to the process by which new blood vessels take shape from existing blood vessels by “sprouting” of endothelial cells, thus expanding the vascular tree. Although angiogenesis and production of angiogenic factors are fundamental for tumor progression in form of growth, invasion, and metastasis [Ribatti, D. and Pezzella, F. Cells (2021) 10 (3): 639], a non-angiogenic form of tumor growth in organs with a dense vascular network, such as the lung, the liver, and the brain has been described [Id, citing Pezzella, F., et al. Am. J. Pathol. (1997) 151: 1417-1423; Pezzella, F., et al. Eur. J. Cancer (1996) 32: 2494-2500; Vermeulen, P B, et al. J. Pathol. (2001) 195: 336-342; Rubenstein, J L, et al. Neoplasia (2000) 2: 306-314; Dome, B, et al. J. Pathol. (2002) 197: 355-362; Holash, J. Science (1999): 284: 1994-1998; Szabo, V., et al. J. Pathol. (2015) 235: 384-396]. Non-angiogenic tumors grow in the absence of angiogenesis by two main mechanism: (1) cancer cells infiltrating and occupying the normal tissues to exploit pre-existing vessels [termed “vascular co-option” or “vessel co-option [Id., citing Vermeulen, P B, et al. J. Pathol. (2001) 195: 336-342]; and (2) the cancer cells themselves form channels able to provide blood flow (termed “vasculogenic mimicry” [Id., citing Donnen, T., et al. Nat. Rev. Cancer (2018) 18: 323-336].

The term “angiogenic factors” as used herein refers to a class of molecules that play a fundamental role in the process of blood vessel formation. Angiogenic factors play a role in regulating angiogenesis. Besides vasculogenic and angiogenic properties, these compounds mediate a complex series of patterning activities during organogenesis. Examples include: VEGF: The VEGF family comprises 5 ligands (VEGFA, B, C, D, and E). VEGF levels reflect the aggressiveness of tumors [Id., citing Aguilar-Cazares, D., et al. Front. Onocl. (2019) 9: 1399]. VEGF overexpression in HCC cells enhances tumor growth and metastasis by promoting angiogenesis. Circulating plasma VEGF levels are elevated in patients with HCC and correlate with high tumor microvessel density (MVD) and poor prognosis [Id., citing Lacin, S. and Yalcin, S. Technol. Cancer Res. Treat. (2020) 19: 1533033820971677]. Binding of VEGFA and VEGFB to VEGF receptor 1 (VEGFR1) leads to the formation of new vessels. The binding of VEGFA, B, C, and D to VEGFR2 stimulates the proliferation and migration of ECs, and angiogenesis. The actions of VEGFC and VEGFD through VEGFR3 result in lymphangiogenesis. VEGFR2 is expressed in almost all ECs and is activated by binding of VEGFA, B, C, or D. VEGFA is the most critical ligand among these VEGFs. The binding of VEGFA/VEGFR2 leads to a phosphorylation cascade that triggers downstream cellular signaling pathways, including the PI3K/AKT and RAF/MAPK pathways, thereby resulting in ECs proliferation and migration, and the formation of branches of new blood vessels necessary for rapid tumor growth and metastasis [Id., citing Apte, R S, et al. Cell (2019) 176: 1248-64, Chen, H., et al. Intl Mol. Sci. (2022) 23: 1475]. The permeability of the newly formed vessels usually increases, thus forming areas of high interstitial pressure and severe hypoxia or necrosis, which further promote HCC progression and angiogenesis [Id., citing Zhu, A X, et al. Nat. Rev. Clin. Oncol. (2011) 8: 292-301].

PDGFs. PDGFs are encoded by 4 genes (PDGFA, B, C, and D) belonging to the cystine knot protein superfamily and are secreted as homodimeric proteins. PDGFs stimulate the growth and migration of glial cells, fibroblasts, and vascular smooth muscle cells [Id., citing Demoulin, J B and Essaghir, A. Cytokine Growth Factor Rev. (2014) 25: 273-283]. PDGFs and PDGF receptors (PDGFRs) are also expressed in a variety of tumors, including HCC [Id., citing Chen, B., et al. Clin. Res. Hepatol. Gastroenterol. (2018) 42: 126-133; Papadopoulos, N. and Lennartsson, J. Mol. Aspects Med. (2018) 62: 75-88]. Activation of the PDGF/PDGFR signaling pathway is correlated with tumor cell proliferation and metastasis via modulation of multiple downstream pathways, including the PI3K/PKB and MAPK/ERK pathways [Id., citing Zou, X., et al. Intl J. Biol. Macromol. (2022) 202: 539-557]. In addition to stimulating cancer cell proliferation, PDGF promotes angiogenesis [Id., citing Tsioumpekou, M., et al. Theranostics (2020) 10: 1122-1135]. In HCC, elevated PDGFR-α levels correlate with MVD and poorer prognosis [Id., citing Shah, A A., et al. Curr. Drug Metab. (2021) 22: 50-59]. Li, L., et al. [Id., citing Cytokine (2021) 141: 155436] have reported that the miR-325-3p-regulated CXCL17/CXCR8 axis in HCC cells regulates PDGF expression and consequently affects angiogenesis. At the molecular level, NUPR1 enhances PDGFA expression in HCC cells, and the released PDGFA facilitates angiogenesis via the PDGFA/MEK/ERK cascade in ECs [Id., citing Chen, C Y, et al. Theranostics (2019) 9: 2361-2379].

FGFs. FGFs are heparin-binding growth factors. [Id., citing Presta, M., et al. Pharmacol. Ther. (2017) 179: 171-187]. The FGF family consists of 22 members including 18 ligands and 4 homologous factors. The FGF1, FGF2, FGF4, and FGF8 subfamilies are the most frequently investigated FGFs in the angiogenic process of HCC. Among these factors, FGF2 is the best known and researched. FGF2 is expressed in HCC cells but is scarcely detectable in nonparenchymal cells or noncancerous liver tissue. It interacts mainly with its receptor, FGFR1, and subsequently mediates angiogenesis through the RAF/MAPK pathway [Id., citing Wang, Y, et al. Cancers (Basel) (2021) 13: 1360]. FGF2 plays multiple roles in various stages of angiogenesis [Id., citing Lieu, C. et al. Clin. Cancer Res. (2011) 17: 6130-6139]. FGF2 not only recruits various host cells to the tumor microenvironment (TME) but also enhances VEGFA-dependent neovascularization during tumor progression—a process essential for subsequent tumor growth and metastasis [Id., citing Pailotta, M T, et al. J. Cell Sci. (2020) 133: 250449]. FGF2 and VEGFA are associated with increased capillarization of sinusoids during angiogenesis in HCC30, and FGF upregulates integrin expression, which in turn alters the cellular state of ECs during angiogenesis.

Angiopoietin. Angiopoietin-1 (Ang1) and -2 (Ang2) are ligands of the tyrosine kinase receptor Tie2, which is expressed on ECs and promotes angiogenesis [Id., citing Bupathi, M., et al. Onco Targets Ther. (2014) 7: 1927-1932]. Ang1 and Ang2 are highly homologous and have similar binding affinity toward Tie2. Ang1 is a widely expressed pro-angiogenic factor in adult tissues that regulates the stabilization and maturation of newly formed vessels by enhancing endothelial cell-to-cell junctions and recruiting pericytes and smooth muscle cells [Id., citing Vanderborght, B., et al. Cells (2020) 9: 2382]. In contrast, Ang2 is generally expressed during vascular remodeling processes [Id., citing Akwii, R G, et al. Cells (2019) 8: 471]. Bupathi et al. reported that Ang2 expression increases in liver cirrhosis and is further elevated in HCC, thus indicating that the angiopoietin pathway is involved in HCC angiogenesis. [Bupathi, M., et al. Onco Targets Ther. (2014) 7: 1927-1932]. Ang2, but not Ang1, significantly increases from early-stage to advanced-stage HCC, and has a high predictive power for overall survival (OS) and progression-free survival (PFS) [Id., citing Choi, G H, et al. World J. Gastroeterol. (2021) 27: 4453-4467]. Generally, Ang2 antagonizes the effect of Ang1 and induces vessel regression in tumors in the absence of VEGFA. Although Ang2 attenuates vascular integrity, it stimulates EC proliferation and migration in the presence of VEGF signaling [Id., citing Roskar, L., et al. Biomolecules (2021) 12: 7]. Co-overexpression of Ang2 and VEGF in HCC results in markedly greater tumor development and angiogenesis, and less intratumoral apoptosis and vessel maturation, than observed with overexpression of either Ang2 or VEGF alone. In addition, inhibition of VEGF signaling abolishes the effects of Ang2 and VEGF co-overexpression, thus indicating that Ang2 synergistically enhances VEGF-mediated HCC development and angiogenesis [Id., citing Yoshiji, H., et al. Gut (2005) 54: 1768-1775].

The terms “apoptosis” or “programmed cell death” refer to a highly regulated and active process that contributes to biologic homeostasis comprising a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways.

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to death domain (DD) containing receptors like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligomerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits inhibitor of apoptosis (IAP) proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome c is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation, can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. [Loberg, R D, et al., J. Biol. Chem. (2002) 277 (44): 41667-41673]. It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “aqueous” is to be understood in the meaning that the pharmaceutical composition contains water as a solvent, whereby also one or more additional solvents may be optionally present.

In pharmacology, the term “AUC” or “area under the curve” of a plot of plasma concentration of a drug versus time after dosage provides an estimate of the overall exposure to the drug and is meaningful for assessing the net pharmacologic response to a given dose of drug [Scheff, J D, et al. Pharm. Res. (2011) 28 (5): 1081-1089, citing Krzyzanski, W and Jusko, W J. J. Pharm. Sci. (1998) 87 (1): 67-72].

The term “autophagy” as used herein refers to a cellular degradation and recycling process that is highly conserved in all eukaryotes. In mammalian cells, there are three primary types of autophagy: microautophagy, macroautophagy, and chaperone-mediated autophagy (CMA). While each is morphologically distinct, all three culminate in the delivery of cargo to the lysosome for degradation and recycling [Parzych, K R and Klionsky, D J. Antioxid. Redox Signal. (2014) 20 (30: 460-73, citing Yang, Z. and Klionsky, DJ. Curr. Opin. Cell Biol. (2010) 22: 124-131). During microautophagy, invaginations or protrusions of the lysosomal membrane are used to capture cargo [Id., citing Mijaljica, D. et al. Autophagy (2011) 7: 673-682]. Uptake occurs directly at the limiting membrane of the lysosome and can include intact organelles. CMA differs from microautophagy in that it does not use membranous structures to sequester cargo but instead uses chaperones to identify cargo proteins that contain a particular pentapeptide motif, these substrates are then unfolded and translocated individually directly across the lysosomal membrane [Id., citing Massey, A., et al. Intl. J. Biochem. Cell Biol. (2004) 36: 2420-2434]. In contrast to microautophagy and CMA, macroautophagy involves sequestration of the cargo away from the lysosome. In this case, de novo synthesis of double-membrane vesicles—autophagosomes—is used to sequester cargo and subsequently transport it to the lysosome [Id., citing Yorimitsu, T. and Klionsky, DJ. Cell Death Differ. (2005) 12: 1542-1552].

The term “binding” and its other grammatical forms as used herein means a lasting attraction between chemical substances.

The term “binding specificity” as used herein involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “bioavailability” and its various grammatical forms as used herein mean the rate and extent to which an active becomes available at the site of action in vivo. It can be a direct reflection of absorption. Bioavailability is defined as the fraction of the originally administered drug that arrives in systemic circulation and depends on the properties of the substance and the mode of administration. Generally, poor solubility of a drug leads to low absorption, low bioavailability, and to challenges with metabolism or permeability.

The term “biocompatible” as used herein refers to a material that is generally non-toxic to the recipient and does not possess any significant untoward effects to the subject and, further, that any metabolites or degradation products of the material are non-toxic to the subject. Typically, a substance that is “biocompatible” causes no clinically relevant tissue irritation, injury, toxic reaction, or immunological reaction to living tissue.

The term “biodegradable” as used herein refers to a material that will erode to soluble species or that will degrade under physiologic conditions to smaller units or chemical species that are, themselves, non-toxic (biocompatible) to the subject and capable of being metabolized, eliminated, or excreted by the subject.

The term “biomarker” (or “biosignature”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes). In evaluating potential therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community. A “predictive biomarker” is a biomolecule that indicates therapeutic efficacy, i.e., an interaction that exists between the biomolecule and therapy that impacts patient outcome. A “prognostic biomarker” is a biomolecule that indicates patient survival independent of the treatment received. It is an indicator of innate tumor aggressiveness.

The term “Cₘₐₓ” as used herein refers to the highest concentration of a drug in the blood, cerebrospinal fluid, or target organ after a dose is given.

The term “capillary” as used herein refers to the smallest type of blood vessel, which is involved in the exchange of fluids and gases between tissues and the blood. A capillary connects an arteriole (small artery) to a venule (small vein) to form a network of blood vessels in almost all parts of the body. The wall of a capillary is thin and leaky.

The term “capillarization” as used herein refers to the formation and development of a network of capillaries in a part of the body.

The term “carrier” as used herein describes a material that does not cause significant irritation to an organism and does not abrogate the biological activity and properties of the active compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials known in the art which are nontoxic and do not interact with other components.

The term “Child-Pugh Score” (also referred to as the Child-Pugh classification, the Child-Turcotte-Pugh (CTP) calculator, and the Child Criteria) as used herein refers to a system for assessing the prognosis of chronic liver disease, primarily cirrhosis. It provides a forecast of the increasing severity of the liver disease and expected survival rate. The score is determined by scoring five clinical measures of liver disease (total bilirubin, serum albumin, prothrombin time, ascites, hepatic encephalopathy) and the possibility of eventual liver failure. A score of 1, 2 or 3 is given to each measure, with 3 being the most severe. Class A is defined as 5-6 points and has a 1-5 year survival rate of 95%. Class B is defined as 7-9 points and has a 1-5 year survival rate of 75%. Class C is defined as 10-15 points and has a 1-5 year survival rate of 50%.

The term “clearance” as use herein at the simplest level refers to a drug's rate of elimination by all routes normalized to the concentration (C) of drug in some biological fluid. Thus, when clearance is constant, the rate of drug elimination is directly proportional to drug concentration. It indicates the volume of biological fluid such as blood or plasma from which drug would have to be completely removed to account for the elimination and is expressed as a volume per unit of time. Clearance by means of various organs of elimination is additive. Division of the rate of elimination by each organ by a concentration of drug (e.g., plasma concentration) will yield the respective clearance by that organ.

The term “compatible” as used herein refers to components of a composition that are capable of being combined with each other in a manner such that there is no interaction that would substantially reduce the efficacy of the composition under ordinary use conditions.

The terms “complete response” or “complete remission” or “CR” as used herein refer to the disappearance of all signs of cancer in response to treatment. This does not always mean the cancer has been cured.

The term “component” as used herein, is meant to refer to a constituent part, element or ingredient.

The term “composition” as used herein, is meant to refer to a material formed by a mixture of two or more substances.

As used herein, the term “condition” as used herein, is meant to refer to a variety of health states and is meant to include disorders or diseases caused by any underlying mechanism or disorder.

As used herein, the term “contact” and its various grammatical forms is meant to refer to a state or condition of touching or of immediate or local proximity. Contacting a composition to a target destination may occur by any means of administration known to the skilled artisan.

The term “costimulation” as used herein refers to the second signal required for completion of lymphocyte activation and prevention of anergy, which is supplied by engagement of CD28 by CD80 and CD86 (T cells) and of CD40 by CD40 Ligand (B cells).

The term “costimulatory molecule” as used herein refers to molecules that are displayed on the cell surface that have a role in enhancing the activation of a T cell that is already being stimulated through its TCR. For example, HLA proteins, which present foreign antigen to the T cell receptor, require costimulatory proteins which bind to complementary receptors on the T cell's surface to result in enhanced activation of the T cell. Co-stimulatory molecules are highly active immunomodulatory proteins that play a critical role in the development and maintenance of an adaptive immune response (Kaufman and Wolchok eds., General Principles of Tumor Immunotherapy, (2007) Chpt 5, 67-121). The two-signal hypothesis of T cell response involves the interaction between an antigen bound to an HLA molecule and with its cognate T cell receptor (TCR), and an interaction of a co-stimulatory molecule and its ligand. Specialized APCs, which are carriers of a co-stimulatory second signal, are able to activate T cell responses following binding of the HLA molecule with TCR. By contrast, somatic tissues do not express the second signal and thereby induce T cell unresponsiveness (Id.). Many of the co-stimulatory molecules involved in the two-signal model can be blocked by co-inhibitory molecules that are expressed by normal tissue (Id.). In fact, many types of interacting immunomodulatory molecules expressed on a wide variety of tissues may exert both stimulatory and inhibitory functions depending on the immunologic context (Id.). The term “co-stimulatory receptor” as used herein refers to a cell surface receptor on naïve lymphocytes through which they receive signals additional to those received through the antigen receptor, and which are necessary for the full activation of the lymphocyte. Examples are CD30 and CD40 on B cells, and CD27 and CD28 on T cells.

The term “cytokeratin” as used herein refers to proteins of cytoskeletal intermediate filaments, whose main function is to enable cells to withstand mechanical stress. In humans, 20 different cytokeratin isotypes have been identified.

The term “damage-associated molecule patterns” (DAMPs) as used herein refers to endogenous danger molecules that are released from damaged or dying cells, which activate the innate immune system by interacting with pattern recognition receptors (PRRs).

The term “derivative” as used herein means a compound that may be produced from another compound of similar structure in one or more steps. A “derivative” or “derivatives” of a compound retains at least a degree of the desired function of the compound. Accordingly, an alternate term for “derivative” may be “functional derivative.” Derivatives can include chemical modifications of the compound, such as akylation, acylation, carbamylation, iodination or any modification that derivatizes the compound. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives. The imidazole nitrogen of histidine can be derivatized to form N-im-benzylhistidine.

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like. “Assay markers” are measurable components whose presence or absence can be detected and correlated to a particular detectable response.

The term “detectable response” as used herein, is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The terms “disease progression” or “progressive disease” as used herein refers to a cancer that continues to grow or spread.

The term “dissolution rate” as used herein refers to the amount of a drug that dissolves per unit time. The term “inherent dissolution rate” is the dissolution rate of a pure API under constant conditions of surface area, rotation speed, pH and ionic strength of the dissolution medium. Inherent dissolution rate is applicable to the determination of thermodynamic parameters associated with different crystalline phases and their solution-mediated phase transformations, investigation of the mass transfer phenomena during the dissolution process, determination of pH-dissolution rate profiles, and the evaluation of the impact of different pH values and the presence of surfactants on the solubilization of poorly soluble compounds.

The term “dose” as used herein, is meant to refer to the quantity of a therapeutic substance prescribed to be taken at one time.

The term “dose escalation study” as used herein refers to a type of study where enrolled patients receive different doses of an investigational agent to determine the recommended phase 2 dose.

The term “dose limiting toxicities” or “DLTs” as used herein refers to side effects of a treatment that are serious enough to prevent an increase in dose of that treatment.

Dose-effect curves. The intensity of effect of a drug (y-axis) can be plotted as a function of the dose of drug administered (X-axis). (Goodman & Gilman's The Pharmacological Basis of Therapeutics, Ed. Joel G. Hardman, Lee E. Limbird, Eds., 10th Ed., McGraw Hill, New York (2001), p. 25, 50). These plots are referred to as dose-effect curves. Such a curve can be resolved into simpler curves for each of its components. These concentration-effect relationships can be viewed as having four characteristic variables: potency, slope, maximal efficacy, and individual variation.

The location of the dose-effect curve along the concentration axis is an expression of the potency of a drug. Id. For example, if the drug is to be administered by transdermal absorption, a highly potent drug is required, since the capacity of the skin to absorb drugs is limited.

The slope of the dose-effect curve reflects the mechanism of action of a drug. The steepness of the curve dictates the range of doses useful for achieving a clinical effect.

The term “maximal or clinical efficacy” refers to the maximal effect that can be produced by a drug. Maximal efficacy is determined principally by the properties of the drug and its receptor-effector system and is reflected in the plateau of the curve. In clinical use, a drug's dosage may be limited by undesired effects.

Biological variability. An effect of varying intensity may occur in different individuals at a specified concentration or a drug. It follows that a range of concentrations may be required to produce an effect of specified intensity in all subjects.

Lastly, different individuals may vary in the magnitude of their response to the same concentration of a drug when the appropriate correction has been made for differences in potency, maximal efficacy and slope.

The duration of a drug's action is determined by the time period over which concentrations exceed the minimum effective concentration (MEC). Following administration of a dose of drug, its effects usually show a characteristic temporal pattern. A plot of drug effect vs. time illustrates the temporal characteristics of drug effect and its relationship to the therapeutic window. A lag period is present before the drug concentration exceeds the MEC for the desired effect. Following onset of the response, the intensity of the effect increases as the drug continues to be absorbed and distributed. This reaches a peak, after which drug elimination results in a decline in the intensity of the effect that disappears when the drug concentration falls back below the MEC. The therapeutic window reflects a concentration range that provides efficacy without unacceptable toxicity. Generally, another dose of drug can be administered to maintain concentrations within the therapeutic window over time.

The terms “drug load (%)” and “drug loading capacity” are used interchangeably to refer to a ratio of the weight of a drug/active agent in the HPβCD inclusion complex relative to the total weight of the inclusion complex, expressed as a percentage. It reflects the drug content of the inclusion complex.

The term “effective dose” as used herein generally refers to that amount of therapeutic agent sufficient to induce a therapeutic effect. An effective dose may refer to the amount of the therapeutic agent sufficient to delay or minimize the onset of symptoms. An effective dose may also refer to the amount of the therapeutic agent that provides a therapeutic benefit in the treatment or management of a disease, disorder or condition. Further, an effective dose is the amount with respect to a therapeutic agent alone, or in combination with other therapies, that provides a therapeutic benefit in the treatment or management of a disease. An effective dose may also be the amount sufficient to enhance the subject's (e.g., a human's) own immune response. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

For any therapeutic agent described herein the effective dose may be initially determined from preliminary in vitro studies and/or animal models. A therapeutically effective dose may also be determined from human data. The applied dose may be adjusted based on the relative bioavailability and potency of the administered agent. Adjusting the dose to achieve maximal efficacy based on the methods described above and other well-known methods is within the capabilities of the ordinarily skilled artisan.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where an agent's plasma concentration can be measured and related to the therapeutic window, additional guidance for dosage modification can be obtained.

The term “effector cell” as used herein refers to a cell that carries out a final response or function. The main effector cells of the immune system, for example, are activated lymphocytes and phagocytes.

The term “effector functions” as used herein refers to the actions taken by effector cells and antibodies to eliminate foreign entities, and includes, without limitation, cytokine secretion, cytotoxicity, and antibody-mediated clearance.

The term “eligible subject” as used herein refers to a subject that satisfies the requirements to be treated with the therapy of the present disclosure under the professional judgment of the patient's physician. Eligibility criteria may include the subject's age, type and stage of cancer, current health status, medical history, and previous treatments.

The term “endothelial to mesenchymal transition” (“EndMT”) as used herein refers to a complex biological process in which endothelial cells adopt a mesenchymal phenotype displaying typical mesenchymal morphology and functions, including the acquisition of cellular motility and contractile properties. It involves numerous molecular and signaling pathways that are triggered and modulated by multiple and often redundant mechanisms depending on the specific cellular context and on the physiological or pathological status of the cells. Endothelial cells undergoing EndMT lose the expression of endothelial cell-specific proteins such as CD31/platelet-endothelial cell adhesion molecule, von Willebrand factor, and vascular-endothelial cadherin and initiate the expression of mesenchymal cell-specific genes and the production of their encoded proteins including α-smooth muscle actin, extra domain A fibronectin, N-cadherin, vimentin, fibroblast specific protein-1, also known as S100A4 protein, and fibrillar type I and type III collagens. [Piera-Velazquez, S P and Jimenez, SA. Physiological Revs. (2019) 99: 1281-1324].

The term “extracellular matrix” (or “ECM”) as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fribronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached to one or more glycosaminoglycans.

The terms “formulation” and “composition” are used interchangeably herein to refer to a product of the described invention that comprises all active and inert ingredients.

The term “free radical” as used herein refers to a highly reactive and usually short-lived molecular fragment having one or more unpaired electrons. Free radicals are highly chemically reactive molecules. Because a free radical needs to extract a second electron fron a neighboring molecule to pair its single electron, it often reacts with other molecules, which initiates the formation of many more free radical species in a self-propagating chain reaction. This ability to be self-propagating makes free radicals highly toxic to living organisms. Oxidative injury can lead to widespread biochemical damage within the cell. The molecular mechanisms responsible for this damage are complex. For example, free radicals can damage intracellular macromrolecules, such as nucleic acids (e.g., DNA and RNA), proteins, and lipids. Free radical damage to cellular proteins can lead to loss of enzymatic function and cell death. Free radical damage to DNA can cause problems in replication or transcription, leading to cell death or uncontrolled cell growth. Free radical damage to cell membrane lipids can cause the damaged membranes to lose their ability to transport oxygen, nutrients or water to cells.

The term “half-life” or (“t_{1/2}”) for the simplest case (e.g., a one-compartment model) refers to the time it takes for the plasma concentration or the amount of the drug in the body to be reduced by 50%. However, drug concentration in plasma often follows a multiexponential pattern of decline, from which two or more half-life terms may be calculated. For any drug following multi-compartment linear drug disposition, the term “operational multiple dosing half-life” is equal to the dosing interval at steady-state where the maximum concentration at steady-state is twice the maximum concentration found for the first dose and where the fall off to the trough plasma/blood concentration from the maximum plasma/blood concentration at steady-state is consistent with this half-life. [Sahin, S. and Benet, L Z. Pharm Res. (2008) 25 (12): 2869-2877). The term “terminal plasma half-life” as used herein refers to the time required to divide the concentration of a drug in plasma by two after reaching pseudo-equilibrium, [Toutain, P-L and Bousquet-Melou, A. J. Veterinary Pharmacol. & Therapeutics. (2004) 27: 427-439].

The term “hydrophilic” as used herein refers to a material or substance having an affinity for polar substances, such as water.

The term “hydrophobic” as used herein refers to a material or substance having an affinity for nonpolar or neutral substances.

The term “hypoxia” as used herein generally refers to sub-physiologic tissue oxygen levels (<5-10 mm Hg). [Challapalli, A., et al. Clin. Transl. Imagin (2017) 5: 225-253].

The term “IDO” as used herein refers to the enzyme indoleamine 2,3-dioxygenase, which catabolizes tryptophan, an essential amino acid, in order to produce the immunosuppressive metabolite kynurenine.

The term “immune system” as used herein refers to the body's system of defenses against disease, which comprises the innate immune system and the adaptive immune system. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

As used herein, the term “immune checkpoints” refers to the array of inhibitory pathways necessary for maintaining self-tolerance and that modulate the duration and extent of immune responses to minimize damage to normal tissue. In T cells, the ultimate amplitude and quality of the immune response, which is initiated through antigen recognition by the TCR, is regulated by a balance between co-stimulatory and inhibitory signals (immune checkpoints). [Pardoll, D M. Nat. Rev. Cancer (2012) 12(4): 252-264]. Immune checkpoint molecules such as PD-1, PD-L1, CTLA-4 are cell surface signaling receptors that play a role in modulating the T-cell response in the tumor microenvironment. Tumor cells have been shown to utilize these checkpoints to their benefit by up-regulating their expression and activity. With the tumor cell's ability to commandeer some immune checkpoint pathways as a mechanism of immune resistance, it has been hypothesized that checkpoint inhibitors that bind to molecules of immune cells to activate or inactivate them may relieve their inhibition of an immune response. Immune checkpoint inhibitors have been reported to block discrete checkpoints in an active host immune response allowing an endogenous anti-cancer immune response to be sustained. Recent discoveries have identified immune checkpoints or targets, like PD-1, PD-L1, PD-L2, CTLA-4, TIGIT, TIM-3, LAG-3, CCR4, OX40, OX40L, IDO, and A2AR, as proteins responsible for immune evasion.

The term “immune escape” or “immune evasion” as used herein refers to a strategy to evade a host's immune response. It is characterized by the inability of the immune system to eliminate transformed cells prior to and after tumor development. The host's contribution is manifested by its inability to recognize antigens expressed by tumor cells, a phenomenon known as “host ignorance.” It happens because of defects in both the innate and adaptive arms of the immune system. The tumor's contribution is manifested by the adaptation of tumor cells to evade the immune system or by developing a microenvironment that suppresses the immune system. [Qian J. et al. (2011) Immune Escape. In: Schwab M. (eds) Encyclopedia of Cancer. Springer, Berlin, Heidelberg. https://doi.org/10.1007/978-3-642-16483-5_2975].

The term “immune homeostasis” refers to the delicate and finely regulated balance of appropriate immune activation and suppression in tissues and organs, driven by a myriad of cellular players and chemical factors. [da Gama Duarte, J., et al. Immunology and Cell Biology (2018) 96: 497-506]

The term “immunohistochemistry” or “ICH” as used herein refers to methods used to detect an antigen in cells or tissue with the employment of specific primary antibodies. Secondary reagents (e.g., secondary antibody) used to detect antibody binding to the antigen in the cells or tissues are usually linked to an enzyme or fluorescent dye to visualize the antigen.

As used herein, the term “immunosuppressive” and its other grammatical forms refers to suppressing or diminishing an immune response either directly or indirectly.

The term “inclusion complex” as used herein refers to a molecular compound having the characteristic structure of an adduct, meaning an unbonded association of two molecules in which one molecule (the host molecule) spatially encloses another (the guest molecule or drug). The guest molecule is entrapped either totally or in part within the framework of the host molecule using only physical forces. No covalent bonding is involved. Cyclodextrins are typical host molecules and can contain a variety of guest molecules and compounds. The inserted compound of the inclusion complex is considered “complexed” with the cyclodextrin. A compound that is not part of an inclusion complex is considered “alone” or “non-complexed.”

The term “inflammation” as used herein refers to the physiologic process by which vascularized tissues respond to injury. See, e.g., FUNDAMENTAL IMMUNOLOGY, 4th Ed., William E. Paul, Ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “integrin” as used herein refers to a family of widely expressed heterodimeric transmembrane adhesion glycoproteins comprising noncovalently bound α and β subunits that link the ECM to a cell's cytoskeleton. Integrins function as mechanotransducers and can transform mechanical forces created by the ECM or the cytoskeleton into chemical signals; they are the primary ECM receptors mediating ECM remodeling. Tumors leverage ECM remodeling to create a microenvironment that promotes tumorigenesis and metastasis. CAFs mediate ECM remodeling and cause ECM stiffness and degradation [Najafi, M., et al. J. Cell Biochem. (2019) 120 (30: 2782-2790). In response to changes in the ECM, integrin signaling also regulates many other interrelated cellular processes including proliferation, survival, cell migration and invasion. Quiescent endothelial cells are anchored to the basement membrane, a structured layer of ECM composed mainly of laminin and collagen type IV. In contrast, growing (angiogenic) endothelial cells are surrounded by provisional ECM containing fibrin, vitronectin, fibronectin and partially degraded collagens. Growth factors upregulate the expression of dimeric integrin receptors (αvβ3, αvβ5, α1β1, α5β1), which recognize specific motifs in ECM molecules (often the RGD (arginine-glycine-aspartic) sequence). Angiogenic growth factor receptors require integrin interactions for their signaling function. [Rak. J. In Chapter 15, Hematology 7^{th} Ed., Hoffman, R., Ben, Jr., E J, Silberstein, L E, Heslop, H E, Weitz, J I, Anastasi, J., Salama, M E, and Abutalib, S A, Eds., Elsevier, Inc. (2018): p. 154-155].

The term “isomer” as used herein refers to one of two or more molecules having the same number and kind of atoms and hence the same molecular weight but differing in chemical structure. Isomers may differ in the connectivities of the atoms (structural isomers), or they may have the same atomic connectivities but differ only in the arrangement or configuration of the atoms in space (stereoisomers). Stereoisomers may include, but are not limited to, E/Z double bond isomers, enantiomers, and diastereomers. Structural moieties that, when appropriately substituted, can impart stereoisomerism include, but are not limited to, olefinic, imine or oxime double bonds; tetrahedral carbon, sulfur, nitrogen or phosphorus atoms; and allenic groups. Enantiomers are non-superimposable mirror images. A mixture of equal parts of the optical forms of a compound is known as a racemic mixture or racemate. Diastereomers are stereoisomers that are not mirror images. The invention provides for each pure stereoisomer of any of the compounds described herein. Such stereoisomers may include enantiomers, diasteriomers, or E or Z alkene, imine or oxime isomers. The invention also provides for stereoisomeric mixtures, including racemic mixtures, diastereomeric mixtures, or E/Z isomeric mixtures. Stereoisomers can be synthesized in pure form (Nógrádi, M.; Stereoselective Synthesis, (1987) VCH Editor Ebel, H. and Asymmetric Synthesis, Volumes 3-5, (1983) Academic Press, Editor Morrison, J.) or they can be resolved by a variety of methods such as crystallization and chromatographic techniques (Jaques, J.; Collet, A.; Wilen, S.; Enantiomer, Racemates, and Resolutions, 1981, John Wiley and Sons and Asymmetric Synthesis, Vol. 2, 1983, Academic Press, Editor Morrison, J). In addition, the compounds of the described invention may be present as enantiomers, diasteriomers, isomers or two or more of the compounds may be present to form a racemic or diastereomeric mixture.

The term “Kaplan-Meier survival curve” as used herein refers to the probability of surviving in a given length of time while considering time in many small intervals. It is commonly used to analyze time-to-event (survival) data, such as the time until death or the time until a specific event occurs. Time is plotted on the x-axis and the survival rate is plotted on the y-axis. Each subject is characterized by three variables: (1) their serial time; (2) their status at the end of their serial time (occurrence of an event of interest or censored); and (3) the study group they are in. “Serial time” refers to the clinical course duration for each subject having a beginning and an end along the timeline of the complete study. An “interval”, which is graphed as a horizontal line, is the serial time duration of known survival. An interval therefore is terminated only by the event of interest. “Censoring” means the total survival time for that subject cannot be accurately determined; this can happen when something negative for the study occurs, such as the subject drops out, is lost to follow-up, or required data is not available, or, conversely, something good happens, such as the study ends before the subject had the event of interest occur. Censoring can occur within the study or terminally at the end. Censored subjects are indicated as tick marks; these do not terminate the interval. [Rich, J T, et al. Otolaryngol. Head Neck Surg. (2010) 143 (3): 331-336]. The Kaplan Meier plot assumes that: (i) at any time subjects who are censored (i.e., lost) have the same survival prospects as subjects who continue to be followed; (ii) the survival probabilities are the same for subjects recruited early and late in the study; and (iii) the event (e.g., death) happens at the time specified. Probabilities of occurrence of an event are computed at a certain point of time with successive probabilities multiplied by any earlier computed probabilities to get a final estimate. The survival probability at any particular time is calculated as the number of subjects surviving divided by the number of subjects at risk. Subjects who have died, dropped out, or have been censored from the study are not counted as at risk.

The term “lethal dose 10” or “LD10” as used herein refers to a statistically derived maximum dose at which 10% of a group of organisms would be expected to die.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions.

Two broad classes of lymphocytes are recognized: the B-lymphocytes (B-cells), which are precursors of antibody-secreting cells, and T-lymphocytes (T-cells).

B-lymphocytes. B-lymphocytes are derived from hematopoietic cells of the bone marrow. A mature B-cell can be activated with an antigen that expresses epitopes that are recognized by its cell surface. The activation process may be direct, dependent on cross-linkage of membrane immunoglobulin (Ig) molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses. (Paul, W. E., “Chapter 1: The immune system: an introduction,” Fundamental Immunology, 4^{th} Edition, Ed. Paul, W. E., Lippicott-Raven Publishers, Philadelphia (1999)).

T-lymphocytes. T-lymphocytes derive from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. (Paul, W. E., “Chapter 1: The immune system: an introduction,” Fundamental Immunology, 4^{th} Edition, Ed. Paul, W. E., Lippicott-Raven Publishers, Philadelphia (1999)).

T cells recognize antigens on the surface of other cells and mediate their functions by interacting with, and altering, the behavior of these antigen-presenting cells (APCs). APCs display three types of protein molecules on their surface that have a role in activating a T cell to become an effector cell: (1) Major histocompatibility complex (MIC) proteins, which present foreign antigen to the T cell receptor; (2) costimulatory proteins which bind to complementary receptors on the T cell surface; and (3) cell-cell adhesion molecules, which enable a T cell to bind to the antigen-presenting cell (APC) for long enough to become activated. (“Chapter 24: The adaptive immune system,” Molecular Biology of the Cell, Alberts, B., et al., Garland Science, NY, 2002).

T-cells are subdivided into two distinct classes based on the cell surface receptors they express. The majority of T cells express T cell receptors (TCR) consisting of α and β chains. A small group of T cells express receptors made of γ and δ chains. Among the α/β T cells are two sublineages: those that express the coreceptor molecule CD4 (CD4+ T cells); and those that express CD8 (CD8+ T cells). CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated. CD8+ T cells can develop into cytotoxic T-lymphocytes (CTLs) capable of efficiently lysing target cells that express antigens recognized by the CTLs.

Suppressor or Regulatory T (Treg) cells. A controlled balance between initiation and downregulation of the immune response is important to maintain immune homeostasis. Both apoptosis and T cell anergy (a tolerance mechanism in which the T cells are intrinsically functionally inactivated following an antigen encounter (Schwartz, R. H., “T cell anergy,” Annu. Rev. Immunol. (2003) 21: 305-334) are important mechanisms that contribute to the downregulation of the immune response. A third mechanism is provided by active suppression of activated T cells by suppressor or regulatory CD4+T (Treg) cells.

The term “macrophage” as used herein refers to a mononuclear, actively phagocytic cell arising from monocyte stem cells in the bone marrow. These cells are widely distributed in the body and vary in morphology and motility. Phagocytic activity is typically mediated by serum recognition factors, including certain immunoglobulins and components of the complement system, but also may be nonspecific. Macrophages also are involved in both the production of antibodies and in cell-mediated immune responses, particularly in presenting antigens to lymphocytes. They secrete a variety of immunoregulatory molecules. Macrophages have been classified based on their mode of activation: classically activated/M1 macrophages respond to interferon-gamma (IFN-7) by releasing pro-inflammatory cytokines and are involved in TH1 cell mediated resolution of acute infection. Alternatively activated/M2 macrophages respond to cytokines from TH2 cells and are involved in wounding and fibrosis. [Ghajar, C M, et al., “The role of the microenvironment in tumor initiation, progression, and metastasis,” In Mendelsohn, J, et al., the Molecular Basis of Cancer, Elsevier Saunders, Philadelphia, citing Pollard, J W. Nat. Rev. Immunol. (2009) 9: 259-270]. The diverse functions of macrophages are executed in a tissue- and context-specific fashion by a number of discrete macrophage subtypes, which aid these developmental processes by remodeling collagen and secreting a host of pro-angiogenic, pro-inflammatory and matrix-degrading factors (Id., citing Qian, B Z, Pollard, J W. Cell (2010) 141: 39-51).

The abbreviation “MAPK” as used herein refers to Mitogen-Activated Protein Kinase (MAPK) signaling, which activates a three-tiered cascade with MAPK kinase kinases (MAP3K) activating MAPK kinases (MAP2K) and finally a MAP kinase (MAPK). MAPKs are protein Ser/Thr kinases that convert extracellular stimuli into a wide range of cellular responses. (Cargnello, M. and Roux, PP, Microbiol. Mol. Biol. Rev. (2011) 75(1): 50-83). The major MAPK pathways involved in inflammatory diseases are extracellular regulating kinase (ERK), p38 MAPK, and c-Jun NH2-terminal kinase (INK). Upstream kinases include TGFβ-activated kinase-1 (TAK1) and apoptosis signal-regulating kinase-1 (ASK1). Downstream of p38 MAPK is MAPK activated protein kinase 2 (MAPKAPK2 or MK2).

The term “maximum tolerated dose” or “MTD” as used herein refers to the highest dose of a drug that does not produce unacceptable toxicity.

The term “mediated” and its various grammatical forms as used herein refers to depending on, acting by or connected through some intervening agency.

The terms “minimum effective concentration”, “minimum effective dose”, or “MEC” are used interchangeably to refer to the minimum concentration of a drug required to produce a desired pharmacological effect in most patients.

The term “modulate” as used herein means to regulate, alter, adapt, or adjust to a certain measure or proportion.

The term “myeloid” as used herein means of or pertaining to bone marrow. The term “myeloid cell” as used herein refers to any white blood cell other than lymphocytes.

The term “myeloid suppressor cells” or “myeloid-derived suppressor cells”, or “MDSCs” as used herein refers to a heterogeneous population of cells characterized by myeloid origin, immature state, and ability to potently suppress T cell responses. These cells regulate immune responses and tissue repair in healthy individuals and the population rapidly expands during inflammation.

The term “natural killer cell” or “NK cell” as used herein refers to a type of innate lymphoid cell important in innate immunity to viruses and other intracellular pathogens and in antibody-dependent cell-mediated cytotoxicity (ADCC). NK cells express activating and inhibitory receptors, but not the antigen-specific receptors of T or B cells.

The term “necrosis” as used herein refers to an irreversible insult that interferes with a vital structure or function of an organelle (plasma membrane, mitochondria, etc.) of a cell and does not trigger apoptosis. Such insults include infectious agents (e.g., bacteria, viruses, fungi, parasites), oxygen deprivation or hypoxia, and extreme environmental conditions such as heat, radiation, or exposure to ultraviolet irradiation. At the cellular level, necrosis is characterized by cell and organelle swelling, ATP depletion, increased plasma membrane permeability, release of macromolecules and eventually inflammation. The processes by which cells undergo death by necrosis vary according to the cause, organ and cell type. While the best studied is ischemic necrosis of cardiac myocyte, the basic processes involved are comparable to those in other organs. Some of the unfolding events may occur simultaneously; others may be sequential. These are:
(1) interruption of blood supply decreases delivery of oxygen and glucose;
(2) anaerobic glycolysis leads to overproduction of lactate and decreased intracellular pH;
(3) distortion of the activities of ionic pumps in the plasma membrane skews the ionic balance of the cell;
(4) activation of phospholipase A₂ (PLA₂) and proteases disrupts the plasma membrane and cytoskeleton;
(5) the lack of oxygen impairs mitochondrial electron transport, thus decreasing ATP synthesis and facilitating production of reactive oxygen species (ROS);
(6) mitochondrial damage promotes the release of cytochrome c to the cytosol;
(7) the cell dies. [Rubin's Pathology: Clinicopathologic Foundations of Medicine, 5^{th} Ed. McDonald, J M, Michalopoulos, G K, Trojanowski, J Q, Ward, P A, Eds Lippincott Williams & Wilkins, M D (2008). pp. 23-26.]

Types of necrosis include, for example:
liquefactive necrosis (occurs when the rate of dissolution of the necrotic cell is considerably faster than the rate of repair; the polymorphonuclear leukocytes of the acute inflammatory reaction contain potent hydrolases capable of digesting dead cells. [Id.]
coapulative necrosis, which refers to light microscopic alterations in a dead or dying cell including pyknosis (the nucleus becomes smaller and stains deeply basophilic as chromatin clumping continues); karyorrhexis (meaning the pyknotic nucleus breaks up into many smaller fragments scattered about the cytoplasm); and karyolysis (referring to the extrusion of the pyknotic nucleus from the cell or progressive loss of chromatin staining). [Id.]
caseous necrosis (which is characteristic of tuberculosis and attributed to the toxic effects of the microbacterial cell well. In casseous necrosis, the necrotic cells fail to retain their cellular outlines; the dead cells persist indefinitely as amorphous, coarsely granular, eosinophilic debris). [Id.]
fat necrosis, which specifically affects adipose tissue and most commonly results from pancreatitis or trauma; the process begins when digestive enzymes normally found only in the pancreatic duct and small intestine, are released from injured pancreatic acinar cells and ducts into the extracellular spaces. On extracellular activation, these enzymes digest the pancreas itself and surrounding tissues, including adipose cells. [Id.]
fibrinoid necrosis (which is an alteration of injured blood vessels in which insudation and accumulation of plasma proteins cause the wall to stain intensely with eosin. The eosinophila of the accumulated plasma proteins obscures the underlying alterations in the blood vessel making it difficult, if not impossible, to determine whether there truly is necrosis in the vascular wall.) [Id.]
gangrenous necrosis (used to describe ischemic necrosis of the lower limbs, sometimes upper limbs or digits). [Id.]

The term “neutrophil” as used herein refers to the most numerous type of white blood cell in human peripheral blood. Neutrophils are phagocytic cells with a multilobed nucleus and granules that stain with neutral stains that enter infected tissues and engulf and kill extracellular pathogens.

The term “neutrophil extracellular traps” or NETs” as used herein refers to a meshwork of nuclear chromatin that is released into the extracellular space by neutrophils undergoing apoptosis at sites of infection, serving as a scaffold that traps extracellular bacteria to enhance their phagocytosis by other phagocytes.

The term “nuclear factor kappa B” and the abbreviation “NFκB” as used herein refer to a proinflammatory transcription factor that switches on multiple inflammatory genes, including cytokines, chemokines, proteases, and inhibitors of apoptosis, resulting in amplification of the inflammatory response [Barnes, P J, (2016) Pharmacol. Rev. 68: 788-815]. The molecular pathways involved in NF-κB activation include several kinases.

The term “Okuda staging score” as used herein includes parameters related to the liver functional status—albumin, ascites, bilirubin—and related to the tumor stage. It incorporates both cancer-related variables and liver function related variables to determine prognosis [Okuda, K., et al. Cancer (1985) 56: 918-928].

The term “optical rotation” refers to the change of direction of the plane of polarized light to either the right or the left as it passes through a molecule containing one or more asymmetric carbon atoms or chirality centers. The direction of rotation, if to the right, is indicated by either a plus sign (+) or a d−; if to the left, by a minus (−) or an /−. Molecules having a right-handed configuration (D) usually are dextrorotatory, D(+), but may be levorotatory, L(−). Molecules having left-handed configuration (L) are usually levorotatory, L(−), but may be dextrorotatory, D(+). Compounds with this property are said to be optically active and are termed optical isomers. The amount of rotation of the plane of polarized light varies with the molecule but is the same for any two isomers, though in opposite directions.

The term “overall response rate” or “ORR” refers to the proportion of patients who have a partial response (PR) or complete response (CR) to therapy. It does not include stable disease.

The term “overall survival” refers to the length of time from either the date of diagnosis or the start of treatment for a disease, such as cancer, to time of death from any cause.

The term “oxidation” as used herein refers to any reaction in which electrons are transferred. Electrons also may be displaced within a molecule without being completely transferred from it.

The term “parenteral” as used herein refers to a route of administration where the drug or agent enters the body without going through the stomach or “gut” and thus does not encounter the first pass effect of the liver. Examples include, without limitation, introduction into the body by way of an injection (i.e., administration by injection), including, for example, subcutaneously (i.e., an injection beneath the skin), intra-arterially (an injection into an artery); intramuscularly (i.e., an injection into a muscle); intravenously (i.e., an injection into a vein), intrathecally (i.e., an injection into the space around the spinal cord or under the arachnoid membrane of the brain), intraventricular injection, intracisternal injection, or infusion techniques. A parenterally administered composition is delivered using a needle.

The term “partial response” as used herein defined by RECIST or mRECIST criteria refers to at least a 30% decrease in the sum of the target lesions.

The term “pharmaceutical composition” is used herein to refer to a composition that is employed to prevent, reduce in intensity, cure or otherwise treat a target condition or disease.

The term “pharmaceutically acceptable,” is used to refer to the carrier, diluent or excipient being compatible with the other ingredients of the formulation or composition and not deleterious to the recipient thereof. The carrier must be of sufficiently high purity and of sufficiently low toxicity to render it suitable for administration to the subject being treated. The carrier further should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use in animals, and more particularly in humans.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulfuric, nitric, phosphoric, maleic, acetic, salicylic, p-toluene sulphonic, tartaric, citric, methane sulphonic, formic, malonic, succinic, naphthalene-2-sulfonic, and benzene sulfonic. Also, such salts may be prepared as alkaline metal or alkaline earth metal salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The term “pharmacokinetics” or “PK” is the study of how the body of a subject interacts with administered substances for the entire duration of exposure. It attempts to summarize the movement of drugs throughout the body of the subject and the actions of the body of the subject on the drug. Pharmacokinetics generally examines four main parameters: absorption (A), distribution (D), metabolism (M) and excretion (E).

The term absorption” as used herein refers to the process that brings a drug from its administration into the systemic circulation. It affects the speed and concentration at which a drug may arrive at its desired location of effect. The absorption process also often includes liberation or the process by which the drug is released from its pharmaceutical dosage form.

The term “distribution” as used herein refers to how a substance is spread throughout the body. Distribution varies based on the biochemical properties of the drug and the physiology of the individual. In the simplest sense, distribution may be influenced by two main factors: diffusion and convection, which in turn may be influenced by the polarity, size or binding abilities of the drug, the fluid state of the patient (hydration and protein concentration) or the physical characteristics (e.g., size, shape) of the individual To be effective, a drug must reach its designated compartmental destination, described by the volume of distribution, and not be protein-bound to be active. Only free drug can act at its pharmacologically active sites, cross into other fluid compartments, or be eliminated. The term “volume of distribution (Vd)” as used herein refers to the amount of drug in the body divided by the plasma drug concentration

The term “metabolism” as used herein refers to the processing of the drug by the body into subsequent compounds. It often converts the drug into more water-soluble substances that will progress to renal clearance or may be required to convert the drug into active metabolites.

The term “excretion” as used herein refers to the process by which the drug is eliminated from the body.

The term “kinetics” as used herein depicts a drug's half-life. The two major forms of drug kinetics are zero order and first order. The term “zero order kinetics” as used herein refers to a constant rate of metabolism and/or elimination independent of the concentration of a drug. There is a variable half-life that decreases as the overall serum concentrations decrease. In contrast, first order kinetics relies on the proportion of the plasma concentration of the drug; it has a constant (t) with decreasing plasma clearance over time.

PI3K/Akt/mTOR Signaling Pathway. The phosphatidylinositol-3-kinase (PI3K)/Akt and the mammalian target of rapamycin (mTor) signaling pathways are crucial to many aspects of cell growth and survival. [Porta, C., et al., “Targeting PI2K/Akt/mTor signaling in cancer.” Frontiers in Oncology (2014) doi.10.3389/fpmc.2014.00064]. They are so interconnected that they could be regarded as a single pathway that, in turn, heavily interacts with many other pathways, including that of hypoxia inducible factors (HIFs).

PI3Ks constitute a lipid kinase family characterized by the capability to phosphorylate inositol ring 3′-OH group in inositol phospholipids. [Id., citing Fruman, D A, et al., Phosphoinositide kinases. Annu. Rev. Biochem. (1998) 67: 481-507]. Class I PI3Ks are heterodimers composed of a catalytic (CAT) subunit (i.e., p110) and an adaptor/regulatory subunit (i.e., p85). This class is further divided into two subclasses: subclass IA (PI3Kα, β, and δ), which is activated by receptors with protein tyrosine kinase activity, and subclass IB (PI3K7), which is ac-tivated by receptors coupled with G proteins [Id., citing Fruman, D A, et al. Phosphoinositide kinases. Annu. Rev. Biochem. (1998) 67: 481-507].

Activation of growth factor receptor protein tyrosine kinases results in autophosphorylation on tyrosine residues. PI3K is then recruited to the membrane by directly binding to phosphotyrosine consensus residues of growth factor receptors or adaptors through one of the two SH2 domains in the adaptor subunit. This leads to allosteric activation of the CAT subunit. PI3K activation leads to the production of the second messenger phosphatidylinositol-3,4,5-triphosphate (PI3,4,5-P3) from the substrate phosphatidylinositol-4,5-bisphosphate (PI-4,5-P2). PI3,4,5-P3 then recruits a subset of signaling proteins with pleckstrin homology (PH) domains to the membrane, including protein serine/threonine kinase-3′-phosphoinositide-dependent kinase I (PDK1) and Akt/protein kinase B (PKB) (Id., citing Fruman, D A, et al., Phosphoinositide kinases. Annu. Rev. Biochem. (1998) 67: 481-507, Fresno-Vara, J A, et al., PI3K/Akt signaling pathway and cancer. Cancer Treat. Rev. (2004) 30: 193-204). Akt/PKB, on its own, regulates several cell processes involved in cell survival and cell cycle progression.

Akt. Akt (also known as protein kinase B) is a 60 kDa serine/threonine kinase. It is activated in response to stimulation of tyrosine kinase receptors such as platelet-derived growth factor (PDGF), insulin-like growth factor, and nerve growth factor (Shimamura, H, et al., J. Am. Soc. Nephrol. (2003) 14: 1427-1434; Datta K., et al. Mol Cell Biol (1995) 15: 2304-2310; Kulik G, Klippel A, Weber M J, Mol Cell Biol (1997) 17: 1595-1606; Yao R, Cooper G M, Science (1995) 267: 2003-2006). Stimulation of Akt has been shown to be dependent on phosphatidylinositol 3-kinase (PI3-kinase) activity (Fruman D A, et al. Annu Rev Biochem (1998) 67: 481-507; Choudhury G G, et al. Am J Physiol (1997) 273: F931-938; Franke T F, et al. Cell (1995) 81: 727-736; Franke T F, Kaplan D R, Cantley L C. Cell (1997) 88: 435-437).

Akt has been shown to act as a mediator of survival signals that protect cells from apoptosis in multiple cell lines (Brunet A, et al. Cell (1999) 96: 857-868; Downward J, Curr Opin Cell Biol (1998) 10: 262-267). For example, phosphorylation of the pro-apoptotic Bad protein by Akt was found to decrease apoptosis by preventing Bad from binding to the anti-apoptotic protein Bcl-XL (Dudek H, et al. Science (1997) 275: 661-665; Datta S R, et al. Cell (1997) 91: 231-241). Akt was also shown to promote cell survival by activating nuclear factor-kB (NF-kB) (Cardone M H, et al. Science (1998) 282: 1318-1321; Khwaja A, Nature (1999) 401: 33-34) and inhibiting the activity of the cell death protease caspase-9 (Kennedy S G, et al. Mol Cell Biol (1999) 19: 5800-5810).

mTOR signaling pathway: Mechanistic target of rapamycin (mTOR) is an atypical serine/threonine kinase that is present in two distinct complexes. The first, mTOR complex 1 (mTORC1), is composed of mTOR, Raptor, GβL, and DEPTOR and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis, or limit catabolic processes such as autophagy. The small GTPase Rheb, in its GTP-bound state, is a necessary and potent stimulator of mTORC1 kinase activity, which is negatively regulated by its GTPase-activating protein (GAP), the tuberous sclerosis heterodimer TSC1/2. TSC1 and TSC2 are the tumour-suppressor genes mutated in the tumour syndrome TSC (tuberous sclerosis complex). Their gene products form a complex (the TSC1-TSC2 (hamartin-tuberin) complex), which, through its GAP activity towards the small G-protein Rheb (Ras homologue enriched in brain), is a critical negative regulator of mTORC1 (mammalian target of rapamycin complex 1). (Huang, J. Manning B D, Biochem J. (2008) 412(2): 179-190). Most upstream inputs are funneled through Akt and TSC1/2 to regulate the nucleotide-loading state of Rheb. In contrast, amino acids signal to mTORC1 independently of the PI3K/Akt axis to promote the translocation of mTORC1 to the lysosomal surface where it can become activated upon contact with Rheb. This process is mediated by the coordinated actions of multiple complexes, including the v-ATPase, Ragulator, the Rag GTPases, and GATOR1/2. The second complex, mTOR complex 2 (mTORC2), is composed of mTOR, Rictor, GβL, Sin1, PRR5/Protor-1, and DEPTOR. mTORC2 promotes cellular survival by activating Akt, regulates cytoskeletal dynamics by activating PKCα, and controls ion transport and growth via SGK1 phosphorylation. Aberrant mTOR signaling is involved in many disease states

As used herein, the term “paracrine signaling” refers to short range cell-cell communication via secreted signal molecules that act on adjacent cells.

The term “PAMPs” is an abbreviation for pathogen-associated molecular patterns. PAMPS are structural patterns present in components or products common to a wide variety of microbes but not host cells. PAMPS are ligands for pattern recognition molecules (PRMs).

The term “pattern recognition molecules” or “PRMs” as used herein refer to proteins recognizing PAMPs. Soluble PRMs include the collectins, acute phase proteins and NOD proteins. Membrane-bound PRMs are pattern recognition receptors.

The term “pattern recognition receptors” or “PRRs” refers to widely distributed membrane bound PRMs fixed in either the plasma membrane of a cell or in the membranes of its endocytic vesicles. The term PRRs includes toll-like receptors (TLRs) and scavenger receptors. Engagement of PRRs induces pro-inflammatory cytokines.

The term “PD-1” or “programmed cell death protein 1” as used herein refers to an inhibitory receptor expressed on the surface of activated T cells. Its ligands, PD-L1 and PD-L2, are expressed on the surface of DCs or macrophages. PD-1 and its ligands PD-L1/PL-L2 act as co-inhibitory factors that can limit the development of the T cell response. PD-L1 is overexpressed on tumor cells or on non-transformed cells in the tumor microenvironment [Pardoll, DM. Nat. Rev. Cancer (2012) 12: 252-264]. PD-L1 expressed on the tumor cells binds to PD-1 receptors on the activated T cells, which leads to the inhibition of the cytotoxic T cells. These deactivated T cells remain inhibited in the tumor microenvironment.

The term “progression” as used herein refers to the course of disease as it becomes worse or spreads in the body.

The term “progression free survival (PFS) defined by RECIST or mRECIST criteria refers to the time from randomization or beginning of treatment until objective tumor progression i.e., progression of an existing tumor size by 20% or appearance of a new lesion or death.

The term “proliferate” and its various grammatical forms as used herein is meant to refer to the process that results in an increase of the number of cells and is defined by the balance between cell division and cell loss through cell death or differentiation.

The term “proteasome” as used herein refers to a large intracellular multisubunit protease that degrades proteins, producing peptides.

The term “purification” and its various grammatical forms as used herein refers to the process of isolating or freeing from foreign, extraneous, or objectionable elements.

The term “racemate” as used herein refers to an equimolar mixture of two optically active components that neutralize the optical effect of each other and is therefore optically inactive.

The term “RECIST” or “Response Evaluation Criteria In Solid Tumors” as used herein refers to a standard way to measure how well a cancer patient responds to treatment. It is based on whether tumors shrink, stay the same, or get bigger. To use RECIST, there must be at least one tumor that can be measured on x-rays, CT scans, or MRI scans. RECIST criteria are based on the measurement of the longest diameter of a patient's tumor lesions. The types of response a patient can have are a complete response (CR), a partial response (PR), progressive disease (PD), and stable disease (SD). [Eisenhauer, E A, et al. Eur. J. Cancer (2009) 45 (2): 228-247]. Major limitations of RECIST that universally affect the response assessment regardless of tumor types or agents include variability of tumor size measurements and tumoral heterogeneity both within a lesion and among different lesions in a patient. [Nishino, M. A M. Soc'y Clinical Oncol. Edu. Book. (2018) 38: 1019-1029]. The term “modified RECIST (mRECIST) criteria” only concerns hepatocellular carcinoma and only takes into account the viable portion defined as the contrast-enhanced portion of the tumor on hepatic arterial phase images. [Yu, H., et al. BMJ Open (2022) 12 (6): e052294].

The term “recurrent cancer” or “recurrence” means a cancer that has come back, usually after a period of time during which the cancer could not be detected. The cancer may come back to the same place as the primary tumor or to another place in the body.

The term “refractory cancer” or “resistant cancer” means a cancer that does not respond to treatment. The cancer may be resistant at the beginning of treatment or it may become resistant during treatment.

The term “relapse” refers to the return of a disease or the signs and symptoms of a disease after a period of improvement.

The terms “relapse-free survival” (RFS) or “disease-free survival” (DFS) mean the length of time after primary treatment for a cancer ends that the patient survives without any signs or symptoms of that cancer.

The term “release” and its various grammatical forms, in the context of cyclodextrin inclusion complexes, refers to dissolution of an active drug component and diffusion of the dissolved or solubilized species by a combination of the following processes: (1) hydration of the cyclodextrin, (2) diffusion of a solution into the cyclodextrin; (3) dissolution of the drug; and (4) diffusion of the dissolved drug out of the cyclodextrin.

The term “sign” as used herein refers to a healthcare provider's evidence of disease.

The term “SNAIL” (or “Snail”) as used herein refers to a zinc-finger transcription factor that belongs to a larger superfamily known as SNAI and participates in cell differentiation and survival [Ganesan, R., et al. Mol. Oncol. (2016) 10 (5): 663-676, citing Nieto, MA. Nat. Rev. Mol. Cell Biol. (2002) 3: 155-166]. Snail's main action mode is by inducing epithelial-to-mesenchymal transition (EMT) by suppression of E-cadherin transcription, which is responsible for cell adhesion and migratory capabilities [Id., citing Bolos, V., et al. J. Cell Sci. (2003) 116: 499-511]. EMT plays a major role in cancer progression and invasion [Id., citing Choi, Y, et al., Hum. Pathol. (2013) 44: 2581-2589].

The term “solid state defense” as used herein refers to a mechanism whereby a macromolecule binds a radical-generating compound, de-excites an excited state species, or quenches a free radical. The most important solid-state defense in the body is the pigment melanin, which scavenges odd electrons to form stable radical species, thus terminating radical chain reactions. Enzymatic defenses against active free radical species include superoxide dismutase, catalases, and the glutathione reductase/peroxidase system. Superoxide dismutase (SOD) is an enzyme that destroys superoxide radicals. Catalase, a heme-based enzyme which catalyzes the breakdown of hydrogen peroxide into oxygen and water, is found in all living cells, especially in the peroxisomes, which, in animal cells, are involved in the oxidation of fatty acids and the synthesis of cholesterol and bile acids. Glutathione, a tripeptide composed of glycine, glutamic acid, and cysteine that contains a nucleophilic thiol group, is widely distributed in animal and plant tissues. It exists in both the reduced thiol form (GSH) and the oxidized disulfide form (GSSG). In its reduced GSH form, glutathione acts as a substrate for the enzymes GSH-S-transferase and GSH peroxidase, both of which catalyze reactions for the detoxification of xenobiotic compounds, and for the antioxidation of reactive oxygen species and other free radicals.

The term “solid tumor” as used herein refers to an abnormal mass of tissue that usually does not contain cysts or liquid areas. Solid tumors may be benign (a growth that does not invade nearby tissue or spread to other parts of the body) or malignant (meaning to grow in an uncontrolled way; malignant tumors can invade nearby tissues and spread to other parts of the body through the blood and lymph system). Different types of solid tumors are named for the type of cells that form them. Types of solid tumors are sarcomas, carcinomas, and lymphomas; leukemias (cancers of the blood) generally do not form solid tumors.

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent. A “suspension” is a dispersion (mixture) in which a finely divided species is combined with another species, with the former being so finely divided and mixed that it doesn't rapidly settle out. In everyday life, the most common suspensions are those of solids in liquid. Among the acceptable vehicles and solvents that may be employed are water, Ringer's solution, and isotonic sodium chloride solution.

According to the European Pharmacopoeia, the solubility of a compound in water in the range of 15° C. to 25° C. is defined as follows:

### Solvent in mL Per Gram Compound

| | |
|---|---|
| Very readily soluble | <1 |
| Readily soluble | from 1 to 10 |
| Soluble | from >10 to 30 |
| Hardly soluble | from >30 to 100 |
| Poorly soluble | from >100 to 1,000 |
| Very poorly soluble | from >1,000 to 10,000 |
| Water-insoluble | >10,000 |

The term “solubilizing agents” as used herein refers to those substances that enable solutes to dissolve.

A “solution” generally is considered as a homogeneous mixture of two or more substances. It is frequently, though not necessarily, a liquid. In a solution, the molecules of the solute (or dissolved substance) are uniformly distributed among those of the solvent.

The term “solvate” as used herein refers to a complex formed by the attachment of solvent molecules to that of a solute.

The term “solvent” as used herein refers to a substance capable of dissolving another substance (termed a “solute”) to form a uniformly dispersed mixture (solution).

The term “stability” and its various grammatical forms as used herein refers to the capability of a particular formulation to remain within its physical, chemical, microbiological, therapeutic and toxicological specifications.

The term “stable disease” as used herein according to RECIST refers to fitting the criteria neither for progressive disease nor for a partial response.

The terms “subject” or “individual” or “patient” are used interchangeably to refer to a member of an animal species of mammalian origin, including but not limited to, mouse, rat, cat, goat, sheep, horse, hamster, ferret, pig, dog, guinea pig, rabbit and a primate, such as, for example, a monkey, ape, or human.

As used herein, the phrase “subject in need” of treatment for a particular condition is a subject having that condition, diagnosed as having that condition, or at risk of developing that condition. According to some embodiments, the phrase “subject in need” of such treatment also is used to refer to a patient who (i) will be administered a composition of the described invention; (ii) is receiving a composition of the described invention; or (iii) has received at least one a composition of the described invention, unless the context and usage of the phrase indicates otherwise.

Unless otherwise stated, “substantially pure” in reference to an inclusion complex intends a preparation of the inclusion complex that contains about or less than about 0.5% impurity per peak and less than about 2% total impurities, wherein the impurity intends a compound other than an inclusion complex of a compound and the HPBCD. Substantially pure preparations include preparations that contain less than about 0.5% impurity per peak and less than about 2% total impurities.

The term “symptom” as used herein refers to a patient's subjective evidence of disease.

The term “transarterial embolization” or “TAE” as used herein refers to a procedure in which the blood supply to a tumor or an abnormal area of tissue is blocked. The mechanism by which arterial embolization preferentially kills HCC but spares adjacent liver tissues arises from the dual blood supply from the portal vein (PV) and the remaining 25% from the hepatic artery (HA). In contrast, HCC almost exclusively receives its blood supply from the HA. Based on this pattern, embolization has been used to selectively block the arterial blood supply to HCC, causing transient but profound ischemia and depriving HCC of essential oxygen and nutrients, thus killing the tumors. (Lin, 2016). However, because of the heterogeneity of the tumor vessels within HCC, the embolization of the tumor-feeding arteries usually results in different degrees of ischemia and hypoxia, ranging from about 0.1 to about 10 M oxygen, inclusive, in HCC after embolization. [Lin, W H, et al. Proc. Nat'l Acad. Sci. USA (2016) 113 (42): 11937-11942].

The term “transarterial chemoembolization” or “TACE” as used herein refers to a procedure that places chemotherapy and embolic agents into a blood vessel feeding a cancerous tumor to cut off the tumor's blood supply and trap the chemotherapy within the tumor.

The term “transarterial tirapazamine embolization” or “TATE” as used herein refers to a treatment procedure in which transarterial embolization is combined with treatment with tirapazamine.

The term “transdifferentiation” as used herein refers to a process whereby somatic cells are reprogrammed into another lineage without going through an intermediate proliferative pluripotent stem cell stage.

The terms “therapeutic amount”, “effective amount”, an “amount effective”, or “pharmaceutically effective amount” of an active agent are used interchangeably to refer to an amount that is sufficient to provide the intended benefit of treatment. However, dosage levels are based on a variety of factors, including the age, weight, sex, medical condition of the patient, the severity of the condition, the route of administration, and the particular active agent employed. Thus, the dosage regimen may vary widely but can be determined routinely by a physician using standard methods.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED₅₀, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest, reduction, or elimination of the progression of a disease manifestation.

The term “therapeutic window” as used herein is meant to refer to a concentration range that provides therapeutic efficacy without unacceptable toxicity. In a drug context, following administration of a dose of a therapeutic agent/drug, its effects usually show a characteristic temporal pattern. A lag period is present before the drug concentration exceeds the minimum effective concentration (“MEC”) for the desired effect. Following onset of the response, the intensity of the effect increases as the drug continues to be absorbed and distributed. This reaches a peak, after which drug elimination results in a decline in the intensity of the effect that disappears when the drug concentration falls back below the MEC. Accordingly, the duration of a drug's action is determined by the time period over which concentrations exceed the MEC. The therapeutic goal is to obtain and maintain concentrations within the therapeutic window for the desired response with a minimum of toxicity. Drug response below the MEC for the desired effect will be subtherapeutic, whereas for an adverse effect, the probability of toxicity will increase above the MEC. Increasing or decreasing drug dosage shifts the response curve up or down the intensity scale and is used to modulate the drug's effect. Increasing the dose also prolongs a drug's duration of action but at the risk of increasing the likelihood of adverse effects. Accordingly, unless the drug is nontoxic, increasing the dose is not a useful strategy for extending a drug's duration of action.

The term “time to progression” as used herein (TTP) refers to the length of time from the date of diagnosis or the start of treatment for the disease until the disease starts to get worse or spread to other parts of the body.

The term “tolerance” as used herein refers to a failure to respond to a particular antigen. Tolerance to self-antigens is an essential feature of the immune system; when tolerance is lost, the immune system can destroy self-tissue, as happens in autoimmune disease.

The term “toxicity” as used herein refers to the degree to which a substance can harm humans or animals. Acute toxicity involves harmful effects in an organism through a single or short-term exposure.

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s). Treatment includes eliciting a clinically significant response without unacceptable levels of side effects. Treatment also includes prolonging survival as compared to expected survival if not receiving treatment.

The term “Treg” or “regulatory T cells” as used herein refers to effector CD4 T cells that inhibit T cell responses and are involved in controlling immune reactions and preventing autoimmunity. The natural regulatory T cell lineage that is produced in the thymus is one subset. The induced regulatory T cells that differentiate from naïve CD4 T cells in the periphery in certain cytokine environments is another subset. Tregs are most commonly identified as CD3+CD4+CD25+FoxP3+ cells in both mice and humans. Additional cell surface markers include CD39, 5′ Nucleotidase/CD73, CTLA-4, GITR, LAG-3, LRRC32, and Neuropilin-1. Tregs can also be identified based on the secretion of immunosuppressive cytokines including TGF-beta, IL-10, and IL-35. Cell surface molecules CTLA-4, LAG-3, and neuropilin-1 (Nrp1) impair dendritic cell (DC)-mediated conventional T cell activation: CTLA-4 and LAG-3 outcompete CD28 and T cell receptor expressed on conventional T cells for binding to CD80/86 and NMC class II on DCs, and Nrp1 stabilizes DC-Treg contact, thereby preventing antigen presentation to conventional T cells [Ikebuchi, R., et al. Front. Immunol. (2019) doi.org/10.3389/fimmu.2019.01098].

The term “tumor associated macrophages” or “TAMs” as used herein refers to an immunosuppressive macrophage subtype found in the tumor microenvironment that is involved in the progression and metastasis of cancer. TAMs are broadly considered M2-like, which can be further classified into the M2a phenotype (induced by IL-4 or IL-13), M2b phenotype (IL-10 high, IL-12 low) and M2c phenotype (TNF-α low) according to distinct signal stimuli. They produce abundant growth factors, extracellular matrix (ECM) remodeling molecules and cytokines for the regulation of cancer proliferation via noncoding RNAs, exosomes and epigenetics [Yan, S. and Wan, G. The FEBS Journal (2021) 288 (21): 6174-6186, citing Qian, B Z and Pollard, J W. (Cell (2010) 141: 39-51]. Activated M2 macrophages distinctively express arginase 1 (ARG1). TAMs can demonstrate direct inhibition on the cytotoxicity of T-lymphocytes through multiple mechanisms and characteristics of tumor evolution, including immune checkpoint engagement via expression, production of inhibitory cytokines [such as IL-10 and transforming growth factor (TGF)-β] and metabolic activities consisting of depletion of 1-arginine (or other metabolites) and the production of reactive oxygen species (ROS). The suppressive immune response renders cancer cells capable of escaping from immune surveillance.

The terms “tumor burden” and “tumor load” are used interchangeably to refer to the number of cancer cells, the size of a tumor, or the amount of cancer in the body.

The term “tumor grade” as used herein and described in Table 2 refers to how normal or abnormal cancer cells look under a microscope. The more normal the cells look, the less aggressive the cancer and the more slowly it grows and spreads. The more abnormal the cells look, the more aggressive the cancer and the faster it is likely to grow and spread.

TABLE 2
Histologic Grade (G)

| | |
|---|---|
| GX | Grade cannot be accessed |
| G1 | Well differentiated |
| G2 | Moderately differentiated |
| G3 | Poorly differentiated |
| G4 | Undifferentiated |

The term “tumor stage” as used herein refers to the extent of a cancer, meaning how large a tumor is and how far the cancer has spread. A cancer is always referred to by the stage it was given at diagnosis, even if it changes over time. New information about how a cancer has changed over time is added to the original stage. Stages I, II and III indicate cancer is present. The higher the number, the more advanced the cancer is. Stage IV indicates that the cancer has spread to distant parts of the body.

Staging systems for hepatocellular carcinoma (HCC) have not been universally adopted. Table 3 describes one system implemented is the American Joint Committee on Cancer (AJCC) tumor/node/metastasis (TNM) classification system, which takes into account tumor characteristics including the extent/size of the tumor (T), spread to nearby lymph nodes (N); metastasis to distant sites (M) and vascular invasion. Patel, A., Hepatocellular Carcinoma Staging, TNM Classification for Hepatocellular Carcinoma, https://emedicine.medscape.com/article/2007061, accessed Feb. 29, 2024, citing Guideline, National Comprehensive Cancer Network, Hepatocellular Carcinoma. NCCN, accessed Oct. 26, 2023, and American Joint Committee on Cancer. Liver. Amin, M B, et al., Eds. AJCC Cancer Staging Manual. 8^{th} Ed. New York: Springer; 2016].

TABLE 3
TMN Classification for HCC
Primary Tumor (T)

| | |
|---|---|
| TX | Primary tumor cannot be assessed |
| T0 | No evidence of primary tumor |
| T1 | Solitary tumor <2 cm, or >2 cm without vascular invasion |
| T1a | Solitary tumor <2 cm |
| T1b | Solitary tumor >2 cm without vascular invasion |
| T2 | Solitary tumor >2 cm with vascular invasion; or multiple tumors, |
| | none >5 cm |
| T3 | Multiple tumors, at least one of which is >5 cm |
| T4 | Single tumor or tumors of any size involving a major branch of the |
| | portal vein or hepatic vein, or tumor(s) with direct invasion of |
| | adjacent organs other than the gallbladder or with perforation of |
| | visceral peritoneum |

Regional lymph nodes (N)

| | |
|---|---|
| NX | Regional lymph nodes cannot be assessed |
| N0 | No regional lymph node metastasis |
| N1 | Regional lymph node metastasis |

Distant metastasis (M)

| | |
|---|---|
| M0 | No distant metastasis |
| M1 | Distant metastasis |

TABLE 4
Anatomic stage/prognostic groups, TNM classification for HCC

| Stage | T | N | M |
|---|---|---|---|
| 1A | T1a | N0 | M0 |
| 1B | T1b | N0 | M0 |
| II | T2 | N0 | M0 |
| IIIA | T3 | N0 | M |
| IIIB | T4 | N0 | M0 |
| IVA | Any T | N1 | M0 |
| IVB | Any T | Any N | M1 |

Another staging system is the “Barcelona-Clinic-Liver Cancer Staging System” or “BCLC”, a validated system that stratifies HCC patients based on tumor size, extent, liver function and performance status, as shown in Table 5.

TABLE 5
Barcelona-Clinic-Liver Cancer Staging System

| | | | | |
|---|---|---|---|---|
| | Performance | | Okuda | |
| Stage | Status | Tumor Stage | Stage | Liver Function |

A¹: Early HCC

| | | | | |
|---|---|---|---|---|
| A1 | 0 | Single, <5 cm | I | No portal |
| | | | | hypertension, |
| | | | | normal |
| | | | | bilirubin |
| A2 | 0 | Single, <5 cm | I | Portal |
| | | | | hypertension, |
| | | | | normal |
| | | | | bilirubin |
| A3 | 0 | Single, <5 cm | I | Portal |
| | | | | hypertension, |
| | | | | abnormal |
| | | | | bilirubin |
| A4 | 0 | 3 tumors, <3 cm | I-II | Child-Pugh |
| | | | | A-B |
| B¹: | 0 | Large, | I-II | Child-Pugh |
| intermediate | | multinodular | | A-B |
| HCC | | | | |
| C²: | 1-2 | Vascular invasion | I-II | Child-Pugh |
| Advanced | | or extrahepatic | | A-B |
| HCC | | spread | | |
| D³: | 3-4 | Any | I-II | Child-Pugh C |
| End-Stage | | | | |
| HCC | | | | |

¹Stage A and B: all criteria need to be fulfilled
²Stage C: At least one of the following criteria needs to be fulfilled: performance status 1-2 or vascular invasion/intrahepatic spread;
³Stage D: At least one of the following criteria needs to be filled: performance status 3-4 or Okuda Stage III/Child Pugh C

The term “tumor microenvironment” or “TME” as used herein refers to the dynamic and complex ecosystem in which tumor cells exist.

The terms “tumorigenesis” “oncogenesis” and “carcinogenesis” are used interchangeably to refer to the transformation of normal cells into cells-of-origin (COOs) and the development of cells-of-origin into tumors.

The term “Twist1” as used herein refers to a basic helix-loop-helix domain-containing transcription factor. It forms homo- or hetero-dimers in order to bind the Nde1 E-box element and activate or repress its target genes. During development, Twist1 is essential for mesoderm specification and differentiation. Heterozygous loss-of-function mutations of the human Twist1 gene cause several diseases including the Saethre-Chotzen syndrome. The Twist1-null mouse embryos die with unclosed cranial neural tubes and defective head mesenchyme, somites and limb buds. Twist1 is expressed in breast, liver, prostate, gastric and other types of cancers, and its expression is usually associated with invasive and metastatic cancer phenotypes. In cancer cells, Twist1 is upregulated by multiple factors including SRC-1, STAT3, MSX2, HIF-1α, integrin-linked kinase and NF-κB. Twist1 significantly enhances epithelial-mesenchymal transition (EMT) and cancer cell migration and invasion, hence promoting cancer metastasis. Twist1 promotes EMT in part by directly repressing E-cadherin expression by recruiting the nucleosome remodeling and deacetylase complex for gene repression and by upregulating Bmi1, AKT2, YB-1, etc. [Qin, Q., et al. Cell Research (2012) 22: 90-106].

The term “ubiquitin” as used herein refers to a small protein that can be attached to other proteins and functions as a protein interaction module or to target them for degradation by the proteasome.

The term “ubiquitin-proteasome system” or “UPS” as used herein that refers to a quality control system in the cell that involves K48-linked ubiquitination of target proteins that are then recognized by the proteasome for degradation.

The term “ubiquitination” as used herein refers to a process of attachment of one or many subunits of ubiquitin to a target protein, which can mediate either degradation by the proteasome or formation of scaffolds used for signaling, depending on the nature of the linkages.

The term “van der Waals forces” as used herein refers to relatively weak electric forces that attract neutral molecules to one another in gases, in liquefied and solidified gases, and in almost all organic liquids and solids.

The term “viscosity” as used herein refers to the property of a fluid that resists the force tending to cause the fluid to flow. Viscosity is a measure of the fluid's resistance to flow. The resistance is caused by intermolecular friction exerted when layers of fluids attempt to slide by one another. Viscosity can be of two types: dynamic (or absolute) viscosity and kinematic viscosity. Absolute viscosity or the coefficient of absolute viscosity is a measure of the internal resistance. Dynamic (or absolute) viscosity is the tangential force per unit area required to move one horizontal plane with respect to the other at unit velocity when maintained a unit distance apart by a fluid. Dynamic viscosity is usually denoted in poise (P) or centipoise (cP), wherein 1 poise=1 g/cm², and 1 cP=0.01 P. Kinematic viscosity is the ratio of absolute or dynamic viscosity to density. Kinematic viscosity is usually denoted in Stokes (St) or Centistokes (cSt), wherein 1 St=10-4 m²/s, and 1 cSt=0.01 St.

“Water for injection” or “WFI” as used herein refers to a form of sterile water used to deliver medications or drugs to patients intravenously, in making solutions, and as a cleaning agent. The United States Pharmacopeia sets the standards at less than 10 CFU per 100 milliliters of aerobic bacteria, less than 500 parts per billion of total organic carbon and fewer than 0.25 EU per milliliter of endotoxins. The resulting product is used by pharmaceutical manufacturers as well as doctors and other healthcare providers.

As used herein, a “wt %” or “weight percent” or “percent by weight” or “wt/wt %” of a component, unless specifically stated to the contrary, refers to the ratio of the weight of the component to the total weight of the composition in which the component is included, expressed as a percentage.

### EMBODIMENTS

According to one aspect, the present disclosure provides cyclodextrin inclusion complexes of tirapazamine. According to some embodiments, the cyclodextrin host is a 3-cyclodextrin derivative. According to some embodiments the β-cyclodextrin derivative is charged or uncharged. According to some embodiments, the uncharged derivative is a β-cyclodextrin substituted by hydroxypropyl groups (beta-hydroxypropyl cyclodextrin or HPβCD). According to some embodiments, the charged derivative is a β-cyclodextrin substituted by sulfopropylether groups (sylfobutylether-ocyclodextrin, or SBE-3-CD). According to some embodiments, the 3-cyclodextrin derivative comprising the tirapazamine guest is soluble in water. According to some embodiments, the β-cyclodextrin derivative comprising the tirapazamine guest is stable when stored for at least 1 hr, at least 24 hr or at least 48 hr at room temperature. According to some embodiments, the β-cyclodextrin derivative comprising the tirapazamine guest is stable when stored for at least 1 hr, at least 24 hr or at least 48 hr at 5° C.

According to some embodiments, the tirapazamine guest molecule is partially or completely included into the cavity of the host β-cyclodextrin derivative molecule. According to some embodiments, the tirapazamine molecule is partially included into the cavity of the host cyclodextrin molecule. According to some embodiments, the extent that the tirapazamine guest is included into the cavity of the host β-cyclodextrin derivative molecule ranges from about 1% to about 50%, i.e., at least 1%, at least 2%, at least 3%, at least 4%, at least 5%, at least 6%, at least 7%, at least 8%, at least 9%, at least 10% included into the cavity of a host β-cyclodextrin derivative molecule. According to some embodiments, the tirapazamine guest is at least 11%, at least 12%, at least 13%, at least 14%, at least 15%, at least 16%, at least 17%, at least 18%, at least 19%, at least 20% included into the cavity of a host β-cyclodextrin derivative molecule. According to some embodiments, the tirapazamine guest is at least 21%, at least 22%, at least 23%, at least 24%, at least 25%, at least 26%, at least 27%, at least 28%, at least 29%, at least 30% included into the cavity of a host β-cyclodextrin derivative molecule. According to some embodiments, the tirapazamine guest is at least 31%, at least 32%, at least 33%, at least 34%, at least 35%, at least 36%, at least 37%, at least 38%, at least 39%, at least 40% included into the cavity of a host 3-cyclodextrin derivative molecule. According to some embodiments, the tirapazamine guest is at least 41%, at least 42%, at least 43%, at least 44%, at least 45%, at least 46%, at least 47%, at least 48%, at least 49%, at least 50% included into the cavity of a host β-cyclodextrin derivative molecule. According to some embodiments, the tirapazamine guest is at least 60% included into the cavity of a host β-cyclodextrin derivative molecule. According to some embodiments, the tirapazamine guest is at least 70% included into the cavity of a host β-cyclodextrin derivative molecule. According to some embodiments, the tirapazamine guest is at least 80% included into the cavity of a host β-cyclodextrin derivative molecule. According to some embodiments, the tirapazamine guest is at least 90% included into the cavity of a host β-cyclodextrin derivative molecule. According to some embodiments, the tirapazamine guest is at least 95% included into the cavity of a host β-cyclodextrin derivative molecule. According to some embodiments, the tirapazamine guest is fully included into the cavity of the host β-cyclodextrin derivative molecule.

According to some embodiments, the molar ratio of the host β-cyclodextrin derivative to the guest tirapazamine in the inclusion complex ranges from about 14:1 to about 2:1, inclusive, i.e., the molar ratio is about 14:1, about 13:1, about 12:1, about 11:1, about 10:1, about 9:1, about 8:1, about 7:1, about 6:1, about 5:1, about 4:1 about 3:1, or about 2:1. According to some embodiments, the molar ratio of the β-cyclodextrin host to the tirapazamine guest in the inclusion complex is about 2:1.

According to some embodiments, a 1 mg/ml solution of the tirapazamine guest complexed in at least a 1% solution of the substituted β-cyclodextrin is water soluble.

According to some embodiments, the pH of the complexed tirapazamine solution ranges from about pH 5.3 to about pH 6.4, i.e., about pH 5.3, about pH 5.4, about pH 5.5., about pH 5.6, about pH 5.7, about pH 5.8, about pH 5.9, about pH 6.0, about pH 6.1, about pH 6.2, about pH 6.3, or about pH 6.4. According to some embodiments, the pH of the complexed tirapazamine solution in a 1% solution of the substituted β-cyclodextrin is pH 6.0.

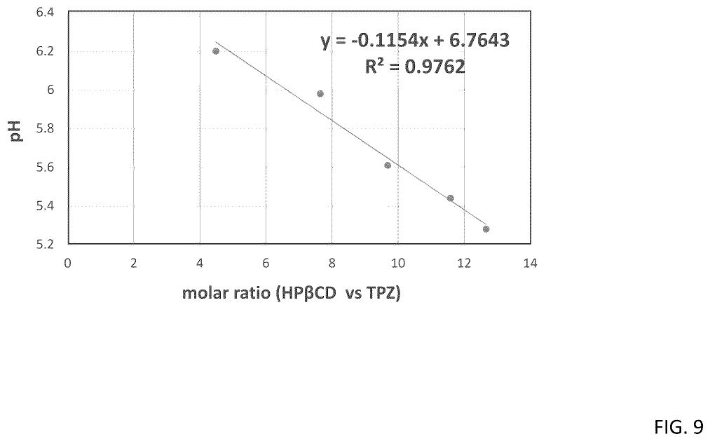

According to some embodiments, the relationship of pH to the molar ratio of the hydroxypropyl β-cyclodextrin-tirapazamine inclusion complex is a straight line that can be described by the relationship y=−0.1154x+6.7643, as shown in FIG. 9.

According to some embodiments, the tirapazamine guest is soluble in a 1% to 25% inclusive aqueous solution of the β-cyclodextrin derivative host at room temperature, i.e., about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% solution of the β-cyclodextrin derivative host. According to some embodiments, solubility of the tirapazamine guest in the 1% to 25%, inclusive, aqueous solution of the β-cyclodextrin derivative host at room temperature ranges from about 1 mg/mL to about 2.55 mg/ml inclusive, i.e., at least 1.05 mg/mL, at least 1.10 mg/mL, at least 1.15 mg/mL, at least 1.20 mg/mL, at least 1.25 mg/mL, at least 1.30 mg/mL, at least 1.35 mg/mL, at least 1.40 mg/mL, at least 1.45 mg/mL, at least 1.50 mg/mL, at least 1.55 mg/mL, at least 1.60 mg/mL, at least 1.65 mg/mL, at least 1.70 mg/mL, at least 1.75 mg/mL, at least 1.80 mg/mL, at least 1.85 mg/mL, at least 1.90 mg/mL, at least 1.95 mg/mL, at least 2.00 mg/mL, at least 2.05 mg/mL, at least 2.10 mg/mL, at least 2.15 mg/mL, at least 2.20 mg/mL, at least 2.25 mg/mL, at least 2.30 mg/mL, 2.35 mg/mL, 2.40 mg/mL, 2.45 mg/mL, 2.50 mg/mL, or 2.55 mg/mL.

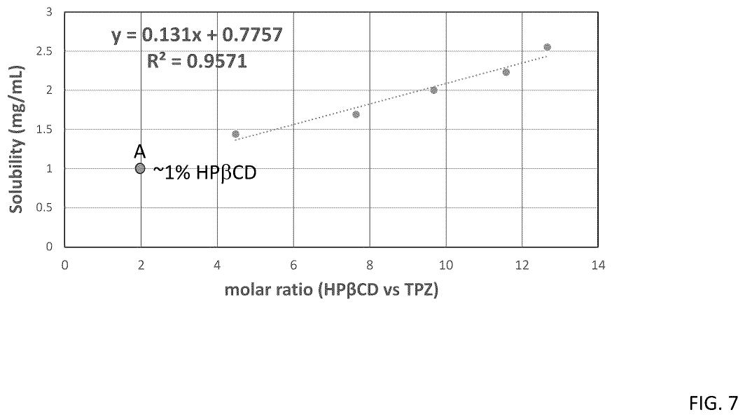

According to some embodiments, the relationship of solubility of the complexed tirapazamine in mg/mL to the molar ratio of the hydroxypropyl β-cyclodextrin:tirapazamine complex is a straight line that can be described by the relationship: y=0.131x+7757 as shown in FIG. 7.

According to some embodiments, solubility of the tirapazamine in a 1% solution of HPβCD at room temperature at a molar ratio of β-cyclodextrin derivative: tirapazamine of 2.0 is about 1 mg/ml at a pH of 6.0.

According to some embodiments, the formed tirapazamine-HPβCD complex may be characterized by one or more techniques. For example, according to some embodiments, HPLC can be used for identification and quantification of inclusion complexes and degradation products.

According to some embodiments, the structure of the tirapazamine-HPβCD may be determined by thermal analysis of the inclusion complex. For example, differential scanning calorimetry (DSC) is a thermoanalytical technique useful in detecting phase transitions in solid samples by measuring the amount of heat absorbed or released during such transitions. According to some embodiments, X-ray diffraction (XRD) patterns can be studied to verify whether complexation causes any structural changes in the TPZ. According to some embodiments, a scanning X-ray diffractometer can be used to obtain X-ray diffraction patterns for TPZ, HPβCD, TPZ-HPβCD complex, and a TPZ-HPβCD physical mixture. According to some embodiments, the drug content of the inclusion complex may be determined from the drug load.

### Cyclodextrin Inclusion Complexes Generally

Cyclodextrins (CDs) are a group of chemically and physically stable macromolecules produced by enzymatic degradation of starch. They are water-soluble and biocompatible in nature, with a hydrophilic outer surface and lipophilic cavity. They have the shape of a truncated cone or torus (ring shape) rather than a perfect cylinder because of the chair conformation of the glucopyranose units, which are linked by α-(1,4) bonds (Gidwani B, Vyas A. Biomed Res Int. 2015; 198268, citing Merisko-Liversidge E, et al. Eur J Pharm Sci. 2003 February; 18(2): 113-20). CDs consist of six or more glucopyranose units.

CDs are classified as natural and derived cyclodextrins. Natural cyclodextrins comprise three well-known, industrially produced (major and minor) cyclic oligosaccharides. The most common natural CDs are a, J, and 7, consisting of 6, 7, and 8 glucopyranose units, respectively (Id., citing Nash R A. Cyclodextrins. In: Wade A, Weller P J, editors. Handbook of pharmaceutical excipients. London: Pharm. Press & Am. Pharm. Assoc.; 1994. p. 145-8), although there is evidence for the natural existence of δ-, ζ-, ξ- and even η-cyclodextrin (9-12 residues) (Id., citing Hirose T, Yamamoto Y. Japanese Patent JP 55480 (2001)).

The main interest in cyclodextrins resides in their ability to form inclusion complexes with several compounds [Id., citing Hedges R A. Chem Rev (1998) 98: 2035-44; Lu X, Chen Y. J. Chromatogr. A (2002) 955: 133-40; Baudin C, et al. Int. J. Environ. Anal. Chem. (2000) 77: 233-42; Kumar R, et al. Bioresour Technol (2001) 28: 209-11; Koukiekolo R, et al. Eur J Biochem (2001) 268: 841-8]. From the X-ray structures it appears that in CDs, the secondary hydroxyl groups (C2 and C3) are located on the wider edge of the ring and the primary hydroxyl groups (C6) on the other edge, and that the apolar C3 and C5 hydrogens and ether-like oxygens are at the inside of the torus-like molecules. This results in a molecule with a hydrophilic outside, which can dissolve in water, and an apolar cavity which provides a hydrophobic matrix, described as a ‘micro heterogeneous environment’ (Id., citing Szetjli J. TIBTRCH (1989) 7: 171-174). As a result of this cavity, CDs are able to form inclusion complexes with a wide variety of hydrophobic guest molecules. One or two guest molecules can be entrapped by one, two or three cyclodextrins (Id.).

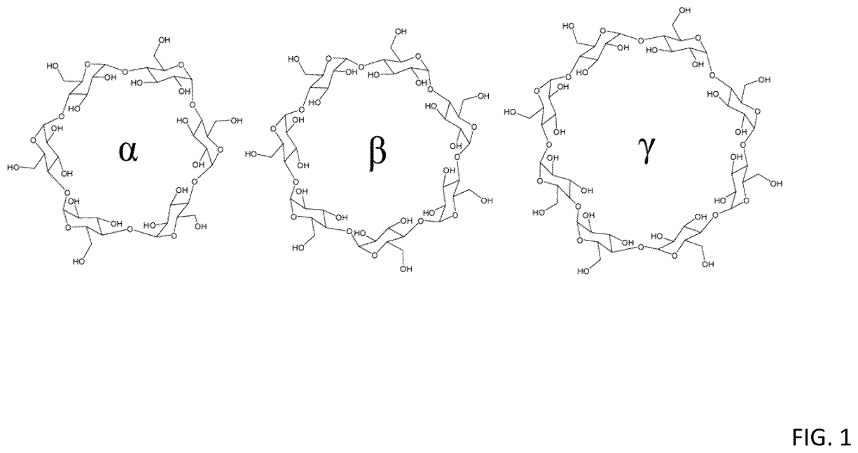

The structural formulae of the three native cyclodextrins is shown in FIG. 1. [Taken from Poulson, B G et al. Polysacccharides (2022) 3 (1): 1-31]. The chair conformation of the D-glucose subunits causes the three native cyclodextrins to form a “truncated” cone instead of a straight symmetrical cylinder. [Id.].

### Properties of Cyclodextrins

The CDs of the three major types: α-cyclodextrin, β-cyclodextrin, and γ-cyclodextrin, are referred to as first generation or parent cyclodextrins. β-Cyclodextrin is the most accessible, the lowest-priced, and generally considered the most useful (Id.). γ-Cyclodextrin is much more soluble in aqueous solutions than β-cyclodextrin, and it possesses relatively good complexing abilities [Loftsson T, Brewster M E. Pharma Tech Eur. 1997; 9: 26-35]. The main properties of the major cyclodextrins are given in Table 6 [Del Valle E M M. Process Biochem. (2004) 39(9): 1033-1046].

TABLE 6
Properties of cyclodextrins

| | α- | β- | γ- |
|---|---|---|---|
| Property | Cyclodextrin | Cyclodextrin | Cyclodextrin |
| Number of glucopyranose | 6 | 7 | 8 |
| units | | | |
| Molecular weight (g/mol) | 972 | 1135 | 1297 |
| Solubility in water at 25° C. | 14.5 | 1.85 | 23.2 |
| (%, w/v) | | | |
| Outer diameter (Å) | 14.6 | 15.4 | 17.5 |
| Cavity diameter (Å) | 4.7-5.3 | 6.0-6.5 | 7.5-8.3 |
| Height of torus (Å) | 7.9 | 7.9 | 7.9 |
| Cavity volume (Å³) | 174 | 262 | 427 |

The natural cyclodextrins have limited aqueous solubility and their complex formation with lipophilic drugs often results in precipitation of solid drug-cyclodextrin complexes. For example, the solubility of β-cyclodextrin in water is only approximately 19 mg/mL or 1.85% (w/v) at room temperature. This low aqueous solubility is, at least partly, associated with strong intramolecular hydrogen bonding in the cyclodextrin crystal lattice. Substitution of any of the hydrogen bond-forming hydroxyl groups, even by hydrophobic moieties such as methoxy groups, will increase the aqueous solubility of β-cyclodextrin [Loftsson T, Brewster M E. Pharma Tech Eur. (1997) 9: 26-35].

Studies of cyclodextrins in solution are supported by a large number of crystal structure studies. Cyclodextrins crystallize in two main types of crystal packing, channel structures and cage structures, depending on the type of cyclodextrin and guest compound [Del Valle E M M. Process Biochem. (2004) 39 (9): 1033-1046]. These crystal structures show that cyclodextrins in complexes adopt the expected ‘round’ structure with all glucopyranose units in the ⁴C₁ chair conformation. Furthermore, studies with linear maltohexoses, which form an antiparallel double helix, indicate that α-cyclodextrin is the form in which the steric strain (meaning the increase in potential energy of a molecule due to repulsion between electrons in atoms that are not directly bonded to each other) due to cyclization is least while γ-cyclodextrin is most strained [Id., citing Szetjli J. Chem Rev (1998) 98: 1743-1753].

Apart from these naturally occurring cyclodextrins, many cyclodextrin derivatives have been synthesized. These derivatives usually are produced by aminations, esterifications or etherifications of primary and secondary hydroxyl groups of the cyclodextrins. Depending on the substituent, the solubility of the cyclodextrin derivatives is usually different from that of their parent cyclodextrins. Virtually all derivatives have a changed hydrophobic cavity volume, and these modifications can improve solubility, stability against light or oxygen, and help control the chemical activity of guest molecules. In addition, as these manipulations frequently produce large numbers of isomeric products, chemical modification can transform the crystalline cyclodextrins into amorphous mixtures, increasing their aqueous solubility and complexity [Loftsson T, Brewster M E. Pharma Tech Eur. (1997) 9: 26-35, citing Pitha J, et al. Intl J Pharm. (1986) 29: 73-82].

The pharmaceutical safety of many of the cyclodextrins currently available has been examined [Id., citing Irie T, Uekama K. J Pharm Sci (1997) 86: 147-162; Fromming K-H, Szejtli. Cyclodextrins in Pharmacy; Kluwer Academic Publishers, Dordrecht, The Netherlands (1994); Duchêne D, Wouessidjewe D. Pharmaceutical and Medical Applications of Cyclodextrins, in S. Dumitriu, Ed., Polysaccharides in Medical Applications; Marcel Dekker, New York, USA, (1996): 575-602]. Topical and oral administration of the parent α-, β- and γ-cyclodextrins, as well as that of their hydrophilic derivatives (for example, 2-hydroxypropyl-β-cyclodextrin, sulfobutylether β-cyclodextrin and maltosyl-β-cyclodextrin) is considered to be safe in most circumstances.

Hydrophilic cyclodextrins poorly penetrate lipophilic biological membranes, meaning that they have negligible oral, dermal or ocular bioavailability [Id., citing Hirayama F, Uekama K. Methods of Investigating and Preparing Inclusion Compounds, in D. Duchêne, Ed., Cyclodextrins and Their Industrial Uses; Editions de Sante, Paris, France, (1987): 131-172]. These materials represent, therefore, true drug carriers. γ-Cyclodextrin, and the hydrophilic β-cyclodextrin derivatives (for example, 2-hydroxypropyl-β-cyclodextrin and probably sulfobutylether β-cyclodextrin) can be used in parenteral dosage forms based on their documented intravenous safety. β-Cyclodextrin and its lipophilic, water-soluble, methylated derivatives cannot be used in parenteral dosage forms. The limited water solubility of β-cyclodextrin causes the compound to precipitate in the kidney, which can induce nephrotoxicity, and the lipophilic cyclodextrins exert detergent-like effects and destabilize biological membranes, including red blood cells (Id.)

Because of their ability to link covalently or noncovalently specifically to other cyclodextrins, cyclodextrins can be used as building blocks for the construction of supramolecular complexes. Their ability to form inclusion complexes with organic host molecules offers possibilities to build supra molecular threads. In this way molecular architectures such as catenanes, rotaxanes, polyrotaxanes, and tubes, can be constructed. Such building blocks, which cannot be prepared by other methods, can be employed, for example, for the separation of complex mixtures of molecules and enantiomers [Del Valle E M M. Process Biochem. (2004) 39 (9): 1033-1046, citing Szetjli J. Chem Rev (1998) 98: 1743-1753].

### Inclusion Complex Formation

The most notable feature of cyclodextrins is their ability to form solid inclusion complexes (host-guest complexes) with a very wide range of solid, liquid and gaseous compounds by a molecular complexation [Id., citing Villiers A. Compt Rendu 1891; 112: 536]. In these complexes, a guest molecule is held within the cavity of the cyclodextrin host molecule. Complex formation is a dimensional fit between host cavity and guest molecule [Id., citing Munoz-Botella S, et al. Ars Pharm (1995) 36: 187-198]. The lipophilic cavity of cyclodextrin molecules provides a microenvironment into which appropriately sized non-polar moieties can enter to form inclusion complexes [Id., citing Loftsson T, Brewster M E. J Pharm Sci (1996) 85: 1017-1025]. No covalent bonds are broken or formed during formation of the inclusion complex (Id., citing Schneiderman E, Stalcup A M. J Chromatogr B (2000) 745: 83-102). The main driving force of complex formation is the release of enthalpy-rich water molecules from the cavity. Water molecules are displaced by more hydrophobic guest molecules present in the solution to attain an apolar-apolar association and decrease of cyclodextrin ring strain resulting in a more stable lower energy state [Id., citing Szetjli J. Chem Rev (1998) 98: 1743-1753].

The binding of guest molecules within the host cyclodextrin is not fixed or permanent but rather is a dynamic equilibrium. Binding strength depends on how well the ‘host-guest’ complex fits together and on specific local interactions between surface atoms. Complexes can be formed either in solution or in the crystalline state, and water is typically the solvent of choice. Inclusion complexation can be accomplished in a co-solvent system and in the presence of any non-aqueous solvent. Cyclodextrin architecture confers upon these molecules a wide range of chemical properties markedly different from those exhibited by non-cyclic carbohydrates in the same molecular weight range (Id.).

Inclusion in cyclodextrins exerts a profound effect on the physicochemical properties of guest molecules as they are temporarily locked or caged within the host cavity giving rise to beneficial modifications of guest molecules, which are not achievable otherwise [Id., citing Schmid G. Trends Biotechnol (1989) 7: 244-248]. These properties include solubility enhancement of highly insoluble guests, stabilization of labile guests against the degradative effects of oxidation, visible or UV light and heat, control of volatility and sublimation, physical isolation of incompatible compounds, chromatographic separations, taste modification by masking off flavors, unpleasant odors and controlled release of drugs and flavors.

The ability of a cyclodextrin to form an inclusion complex with a guest molecule is a function of two key factors. The first is steric and depends on the relative size of the cyclodextrin compared to the size of the guest molecule or certain key functional groups within the guest. If the guest is the wrong size, it will not fit properly into the cyclodextrin cavity. The second critical factor is the thermodynamic interactions between the different components of the system (cyclodextrin, guest, solvent). For a complex to form, there must be a favorable net energetic driving force that pulls the guest into the cyclodextrin (Id.).

In general, there are four energetically favorable interactions that help shift the equilibrium to form an inclusion complex: (1) the displacement of polar water molecules from the apolar cyclodextrin cavity; (2) the increased number of hydrogen bonds formed as the displaced water returns to the larger pool; (3) a reduction of the repulsive interactions between the hydrophobic guest and the aqueous environment; and (4) an increase in the hydrophobic interactions as the guest inserts itself into the apolar cyclodextrin cavity (Id.).

While the initial equilibrium to form the complex is very rapid (often within minutes), the final equilibrium can take much longer to reach. Once inside the cyclodextrin cavity, the guest molecule makes conformational adjustments to take maximum advantage of the weak van der Waals forces that exist (Id.).

Dissociation of the inclusion complex is a relatively rapid process usually driven by a large increase in the number of water molecules in the surrounding environment. The resulting concentration gradient shifts the equilibrium to the left. In highly dilute and dynamic systems like the body, the guest has difficulty finding another cyclodextrin to reform the complex and is left free in solution (Id.).

The central cavity of the cyclodextrin molecule is lined with skeletal carbons and ethereal oxygens of the glucose residues. It is, therefore, lipophilic. The polarity of the cavity has been estimated to be similar to that of aqueous ethanolic solution (Id., citing Fromming K H, Szejtli J. Cyclodextrins in pharmacy. Topics in inclusion science. (1994) Dordrecht: Kluwer Academic Publishers). It provides a lipophilic microenvironment into which suitably sized drug molecules may enter and include.

Cyclodextrin inclusion is a stoichiometric molecular phenomenon in which usually only one guest molecule interacts with the cavity of a cyclodextrin molecule to become entrapped. In the case of some low molecular weight molecules, more than one guest molecule may fit into the cavity, and in the case of some high molecular weight molecules, more than one cyclodextrin molecule may bind to the guest. In principle, only a portion of the molecule must fit into the cavity to form a complex. As a result, one-to-one molar ratios are not always achieved, especially with high or low molecular weight guests. A variety of non-covalent forces, such as van der Waals forces, hydrophobic interactions and other forces, are responsible for the formation of a stable complex (Id.).

### Compositions/Formulations

According to another aspect, a therapeutic amount of the host β-cyclodextrin derivative molecule comprising tirapazamine as a guest can be formulated with a pharmaceutically acceptable carrier to form a pharmaceutical composition. According to some embodiments, the pH of the formulation ranges from about pH 5.2 to about pH 7.

According to some embodiments, the carrier is an aqueous carrier.

Additives that can be used with the inclusion complexes described herein (e.g., an inclusion complex of a compound with a cyclodextrin) may include, for example, one or more excipients, one or more stabilizers, one or more preservatives (e.g., including antimicrobial preservatives), one or more pH adjusting and/or buffering agents, one or more tonicity adjusting agents, one or more thickening agents, one or more suspending agents, one or more binding agents, one or more viscosity enhancing agents, one or more antioxidants, one or more sweetening agents and the like, either alone or together with one or more additional pharmaceutical agents, provided that the additional components are pharmaceutically acceptable. According to some embodiments, the formulation may include combinations of two or more of the additional components as described herein (e.g., any of 2, 3, 4, 5, 6, 7, 8, or more additional components).

According to some embodiments, the additives may include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, polyvinylpyrrolidinone, low melting waxes, ion exchange resins, and the like, as well as combinations of any two or more thereof. Other suitable pharmaceutically acceptable excipients are described in Remington's Pharmaceutical Sciences, Mack Pub. Co., New Jersey 18th edition (1996), Handbook of Pharmaceutical Excipients, Pharmaceutical Press and American Pharmacists Association, 5th edition (2006), and Remington: The Science and Practice of Pharmacy, Lippincott Williams & Wilkins, Philadelphia, 20th edition (2003) and 21st edition (2005).

The pharmaceutical compositions of the described invention can be formulated for parenteral administration, for example, by injection, such as by bolus injection or continuous infusion. The pharmaceutical compositions can be administered by continuous infusion subcutaneously over a predetermined period of time. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents.

For parenteral administration, a pharmaceutical composition can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. The vehicle or lyophilized powder may contain additives that maintain isotonicity (e.g., sodium chloride, mannitol) and chemical stability (e.g., buffers and preservatives). The formulation is sterilized by commonly used techniques. According to some embodiments, the administration of the formulation is coupled with transient hepatic artery ligation (HAL).

The inclusion complexes may also be formulated for topical administration, especially when the target of treatment includes areas or organs readily accessible by topical application, including diseases of the eye, the skin, the lung, or the lower intestinal tract. Suitable topical formulations are readily prepared for each of these areas or organs. Topical application for the lower intestinal tract can be affected in a rectal suppository formulation or in a suitable enema formulation. Topically applied transdermal patches may also be used.

For oral administration, the pharmaceutical compositions can be formulated readily by combining the active agent(s) with pharmaceutically acceptable carriers well known in the art. Such carriers enable the actives of the disclosure to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, alter adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the described invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In addition to the formulations described previously, the compositions of the described invention can also be formulated as a depot preparation. Such long-acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

According to the foregoing embodiments, the pharmaceutical composition may be administered once, for a limited period of time or as a maintenance therapy over an extended period of time, for example until the condition is ameliorated, cured or for the life of the subject. A limited period of time may be for 1 week, 2 weeks, 3 weeks, 4 weeks and up to one year, including any period of time between such values, including endpoints. According to some embodiments, the pharmaceutical composition may be administered for about 1 day, for about 3 days, for about 1 week, for about 10 days, for about 2 weeks, for about 18 days, for about 3 weeks, or for any range between any of these values, including endpoints. According to some embodiments, the pharmaceutical composition may be administered for more than one year, for about 2 years, for about 3 years, for about 4 years, or longer.

Pharmaceutical compositions comprising cyclodextrin inclusion complexes of tirapazamine as disclosed herein also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

According to some embodiments, the pharmaceutical composition improves stability of the tirapazamine when compared to the stability of the non-complexed tirapazamine alone.

According to some embodiments, the pharmaceutical composition reduces toxicity of injected related pain when compared to the toxicity of the non-complexed tirapazamine alone.

According to some embodiments, the pharmaceutical composition delivers a minimum effective concentration of the tirapazamine to locations in vivo to which only a small amount of formulation volume is capable of being administered.

### Methods

According to another aspect, the present disclosure provides a method of treating a liver tumor comprising
(a) targeting the liver tumor by administering a pharmaceutical composition comprising a cyclodextrin inclusion complex comprising a β-cyclodextrin host molecule substituted by hydroxypropyl groups (hydroxypropyl-β-cyclodextrin, or HPβCD) comprising a cavity containing tirapazamine as a guest,
wherein
the carrier is an aqueous carrier;
pH of a 1 mg/ml aqueous solution of the complexed tirapazamine guest ranges from pH 5.3 to 6.4, inclusive;
the tirapazamine guest is at least partially included into the cavity of the 3-cyclodextrin host molecule, wherein the extent of inclusion ranges from about 1% to about 50%, inclusive; and
a molar ratio of the cyclodextrin host to the tirapazamine guest ranges from about 14:1 to about 2:1, inclusive;
(b) transiently ligating the hepatic artery of the subject so that the cyclodextrin inclusion complex comprising tirapazamine is transiently retained within liver tissue comprising the liver tumor; and
(c) producing targeted necrosis within the liver tumor and not viable liver tissue,

According to some embodiments, the pharmaceutical composition comprising the cyclodextrin inclusion complex of tirapazamine reduces toxicity of injection-related pain when compared to non-complexed tirapazamine alone.

According to some embodiments of the method, the transient ligation of the hepatic artery is for a time period of at least about 40 minutes.

According to some embodiments of the method, the administering is intravenously or intra-arterially.

According to some embodiments of the method, the β-cyclodextrin host molecule is substituted by hydroxypropyl groups (hydroxypropyl-β-cyclodextrin, or HPβCD). According to some embodiments of the method, the molar ratio of the cyclodextrin host to the tirapazamine guest is about 2:1. According to some embodiments of the method, a 1 mg/ml solution of the tirapazamine guest complexed with at least a 1% solution of the substituted cyclodextrin host is water soluble.

According to some embodiments of the method, the aqueous carrier is water, normal saline, Ringer's solution or a dextrose solution.

According to some embodiments of the method, immunohistochemical (IHC) techniques can be used to detect tissue biomarkers of hypoxia to immunostain formalin-fixed paraffin-embedded (FFPE) tissue sections using commercial antibodies. [See, e.g., Shidham, V B and Layfield, L J. Cytojournal (2021) 18: 2]. Exemplary markers can include hypoxia markers VEGF and carbonic anhydrase IX (CAIX) [Di Tommaso, L. and Roncalli, M. Front. Medicine (Lausanne) (2017) 4: 10, citing Sciarra, A. et al. Liver Int. (2015) 35: 2466-73; Huang, W-J et al. PLoS One (2015) 10 (3): e0119181], markers to detect TAMS/M2 macrophages CD163, CD204, and CD206 [Rafiul Haque, AQSM, et al. Scientific Reports (2019) 9: 14611], markers of key signaling pathways (e.g., AKT/protein kinase B (PKB), PI3K, GSK-30, GSK-3a, MAK2K, MAP3K, ERK1/2, etc., available, for example, from ThermoFisher), markers of transcription pathways (e.g., Snail, Twist), markers of inflammation (e.g., TNF, IL-1β, IL-6, IL-8), and markers of phosphorylation or activation states.

According to some embodiments of the method, immunohistochemical (IHC) techniques can be used to detect tissue biomarkers of necrosis to immunostain paraffin embedded tissue sections using commercial antibodies. Exemplary markers can include cytokeratin markers [e.g., see Judkins, A R et al. Am. J. Clin. Pathol (1998) 110: 641-646], and damage-associated molecule patterns [see, e.g., Yang, M. et al. Liver Transpl. (2014) 20(11): 1372-1382].

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates, which may need to be independently confirmed.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed.

Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Since β cyclodextrin has low solubility in water, its derivatives 2-hydroxypropyl-β-cyclodextrin (HPβCD) and sulfobutylether-β-cyclodextrin (SBEβCD) were evaluated. A total of six complexing agents were investigated to assess their impact on TPZ: HPβCD, SBEβCD, benzyl alcohol, Nicotinamide, Arginine and Meglumine.

### Materials and Apparatus Used for Examples 1-4

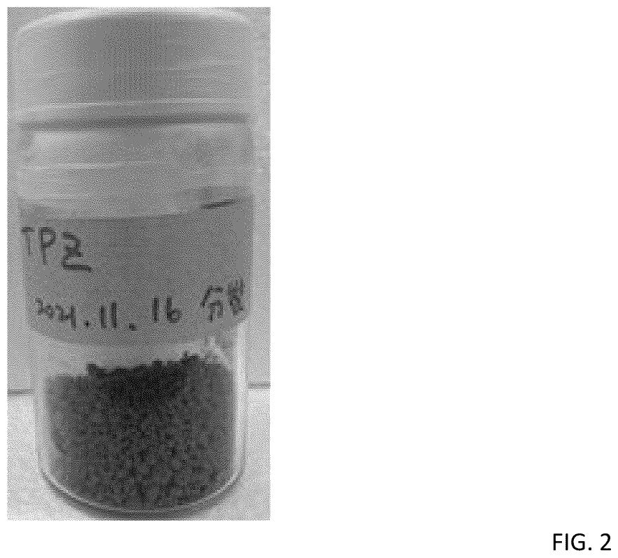

1. Tirapazamine (TPZ). [FIG. 2]
2. HPβCD, hydroxypropyl betacyclodextrin, Batch no. E0181, ROQUETTE
3. SBEβCD, sulfobutylether-β-cyclodextrin, Captisol, Lot no. NC-04A-160149, CyDex
4. BA, benzyl alcohol, Lot no. IEG0931, Spectrum
5. Nicotinamide, Lot no. 111K0026, Sigma
6. Arginine, Lot no. A023418, Acros
7. Meglumine, Lot no. BCCK1203, Sigma
8. WFI, Water for Injection, Taiwan Biotech
9. 0.1 N HCl, Hydrochloric Acid, Lot no. HC268120, Merck
10. Analytical balance, EX225D/AD, OHAUS
11. pH Meter, SevenCompact™ pH/Ion meter S220, METTLER TOLEDO
12. Vortex, VORTEX-GENIE 2, Scientific Industries
13. Ultrasonic Bath, Power sonic 410, HWASHIN
14. 0.2 m Supor filter, PES membrane, PALL

### Example 1: Hydroxypropyl β-Cyclodextrin (HPβCD) as an Excipient to Dissolve Tirapazamine

Low aqueous solubility is a problem encountered during development of an i.v. formulation of tirapazamine (TPZ). Currently, TPZ IV formulations containing 0.7 mg/mL tirapazamine in citrate buffer at pH 4 have been used in early phase clinical studies.

HPβCD is a well-established and safe excipient in both preclinical and clinical applications. In mice, up to 10 gram/kg (by weight) HPβCD has been administered acutely by intra-peritoneal (i.p.) injection. It was neither lethal nor did it produce any toxicity. Following a 1-week intravenous study at a single dose level of 1 g HPβCD, no adverse effects were reported. Intravenous administration of HPβCD was studied in mice, monkeys, rats and dogs after single or repeated doses for up to 90 days. In Cynomolgus monkeys, a single intravenous dose of 10 g/kg of 50% w/v HPβCD was not lethal. In mice, a single intravenous dose of up to 2 g/kg by weight was not lethal. A number of clinical studies showed that HPβCD was well tolerated and safe in the majority of patients receiving HPβCD at daily oral doses of 4-8 g for at least 2 weeks. When higher oral daily doses of 16-24 g were given for 14 days to healthy volunteers, there was an increased incidence of soft stools and diarrhea. Therefore, based on these clinical data, HPβCD was considered to be non-toxic (at least for 14 days) if the daily dose is <16 g.

### Methods

### 1. Solubility of Tirapazamine (TPZ) in 5%, 10%, 15%, 20% and 25% HPβCD:

### 1.1 Test A (Initial):

a. Five different concentrations (5%, 10%, 15%, 20% and 25%) of HPβCD solutions were prepared by dissolving in water and passed through a 0.2 m PES filter.
b. Approximately 10 mg of TPZ were weighed into a glass vial.
c. A small amount of the chosen HPβCD solution was added to the vial, and the mixture was then vortexed vigorously for 1-3 minutes (and may mixed by ultrasonic bath for 5-10 minutes) at room temperature.
d. The mixture was observed by visual inspection to determine whether TPZ dissolves in HPβCD solution.
e. If TPZ did not dissolve, then more HPβCD solution was added; steps c-e were repeated until TPZ was completely dissolved.
f. The total volume of HPβCD solution that made TPZ dissolve was recorded.
g. The above steps were followed to determine the solubility of TPZ in different concentrations of HPβCD solution.

### 1.2 Test B (Stored at Room Temperature):

a. Group A of the TPZ/HPβCD solutions was stored at room temperature away from light and observed to determine if the TPZ precipitated out.
b. For TPZ/HPβCD solutions that precipitated out, more of the HPβCD solution (0.5 mL) was added and then vortexed vigorously for 1-3 min (and may then be ultrasonically mixed for 5-10 min) at room temperature. This step was repeated until the precipitate was completely dissolved.
c. TPZ/HPβCD solutions were stored at room temperature and observed to assess their stability at 1 h, 24 h and 48 h.
d. The pH value of the final solutions was measured.

### 1.3 Test C (Stored at 5° C.):

a. Group B of the TPZ/HPβCD solutions was stored at 5° C. away from light and observed to determine whether the TPZ precipitated out.
b. More HPβCD solution (0.5 mL) was added to those TPZ/HPβCD solutions that precipitated out, then vortexed vigorously for 1-3 min; and then ultrasonic mixed for 5 minutes at room temperature. These steps were repeated until the precipitate was completely dissolved.
c. TPZ/HPβCD solutions were stored at room temperature and observed to assess their stability at 1 h, 24 h and 48 h.
   1.4 Test D: Preparation of 1 mg/mL of TPZ in 5% and 2.5% HPβCD Solutions:
a. 2.5% and 5% HPβCD solutions were prepared by dissolving HPβCD in water and they were then passed through the 0.2 m PES filter.
b. 20 mg of TPZ and 20 mL of 2.5% or 5% HPβCD solution were put into a glass vial, and the mixture was vortexed vigorously for 1 min and then ultrasonic mixed for at least 5 minutes at room temperature to dissolve TPZ.
c. The TPZ/HPβCD solution was separated into two vials and then stored at different temperature (room temperature and 5° C.) for 24 h to observe if it is stable.

### Results

### 1. Solubility of Tirapazamine (TPZ) in 5%, 10%, 15%, 20% and 25% HPβCD:

### 1.1 Test A (Initial):

Results of the solubility testing of TPZ in different concentration HPβCD solutions at initial time are listed in Table 7.

All the TPZ/HPβCD solutions were clear solutions initially and there was no change for the first 3 hours. After 14 hours, precipitates (needle-shaped crystals) were found in all of the TPZ/HPβCD solutions at room temperature and could not be re-dissolved by vigorously vortex mixing.

TABLE 7
Solubility of TPZ in different concentration
HPβCD solutions at initial time.

| | | Vol. of | Solubility, |
|---|---|---|---|
| | | HPβCD | mg/mL |
| Test Sample | TPZ, mg | soln., mL | (initial) |
| TPZ in 25% HPβCD Soln. | 10.18 | 3.5 | ~2.91 |
| TPZ in 20% HPβCD Soln. | 10.04 | 3.5 | ~2.87 |
| TPZ in 15% HPβCD Soln. | 10.00 | 3.5 | ~2.86 |
| TPZ in 10% HPβCD Soln. | 10.11 | 4.5 | ~2.25 |
| TPZ in 5% HPβCD Soln. | 10.05 | 5.5 | ~1.83 |

### 1.2 Test B (Stored at Room Temperature):

The precipitate could be dissolved by adding more of the HPβCD solution, and the TPZ/HPβCD solutions became clear. The appearance of the TPZ/HPβCD solutions did not change after stored at room temperature for 1 h, 24 h and 48 h.

The final solubility and pH values of TPZ in different concentration HPβCD solutions are listed in Table 8.

TABLE 8
Final solubility and pH values of TPZ in
different concentration HPβCD solutions.

| | | Vol. of | | |
|---|---|---|---|---|
| | | HPβCD | | |
| | | soln., | Solubility, | pH |
| Test Sample | TPZ, mg | mL | mg/mL | value |
| TPZ in 25% | 10.18 | 3.5 + 0.5 = 4 | ~2.55 | 5.28 |
| HPβCD Soln. | | | | |
| TPZ in 20% | 10.04 | 3.5 + 1.0 = 4.5 | ~2.23 | 5.44 |
| HPβCD Soln. | | | | |
| TPZ in 15% | 10.00 | 3.5 + 1.5 = 5 | ~2.00 | 5.61 |
| HPβCD Soln. | | | | |
| TPZ in 10% | 10.11 | 4.5 + 1.5 = 6 | ~1.69 | 5.98 |
| HPβCD Soln. | | | | |
| TPZ in 5% | 10.05 | 5.5 + 1.5 = 7 | ~1.44 | 6.20 |
| HPβCD Soln. | | | | |

### 1.3 Test C (Stored at 5° C.):

A few precipitates were found in all the TPZ/HPβCD solutions that were stored at 5° C. at different times (TPZ in 5%, 10%, 15%, 20% and 25% HPβCD solutions each precipitated after 1, 2, 3.5, 3.5 and 21 hours).

The precipitate could be dissolved by adding more of the HPβCD solution, after which the TPZ/HPβCD solutions became clear. The appearance of the TPZ/HPβCD solutions did not change after they were stored at room temperature for 1, 24 and 48 h.

The solubility of TPZ in different concentration HPβCD solutions is listed in Table 9.

TABLE 9
Solubility of TPZ in different concentration
HPβCD solutions for item 1.3 testing.

| Test | TPZ, | Vol. of HPβCD | |
|---|---|---|---|
| Sample | mg | soln., mL | Solubility, mg/mL |
| TPZ in 25% | 10.18 | 3.5 + 0.5 + 0.5 = 4.5 | ~2.26 |
| HPβCD Soln. | | | |
| TPZ in 20% | 10.04 | 3.5 + 1.0 + 0.5 = 5.0 | ~2.01 |
| HPβCD Soln. | | | |
| TPZ in 15% | 10.00 | 3.5 + 1.5 + 0.9 = 5.9 | ~1.69 |
| HPβCD Soln. | | | |
| TPZ in 10% | 10.11 | 4.5 + 1.5 + 0.5 = 6.5 | ~1.56 |
| HPβCD Soln. | | | |
| TPZ in 5% | 10.05 | 5.5 + 1.5 + 0.5 = 7.5 | ~1.34 |
| HPβCD Soln. | | | |

1.4 Preparation of 1 mg/mL of TPZ in 5% and 2.5% HPβCD Solutions:

The experimental setup is shown in Table 10.

TABLE 10
Experimental setup: to assess the appearance of
1 mg/mL of TPZ in 5% and in 2.5% HPβCD solutions

| | 1 mg/mL of TPZ in 2.5% | | 1 mg/mL of TPZ in 5% | |
|---|---|---|---|---|
| Time | HPβCD Solution | | HPβCD Solution | |
| Initial | Room Temperature (RT) | | RT | |
| After 24 hours | 5° C. | RT | 5° C. | RT |

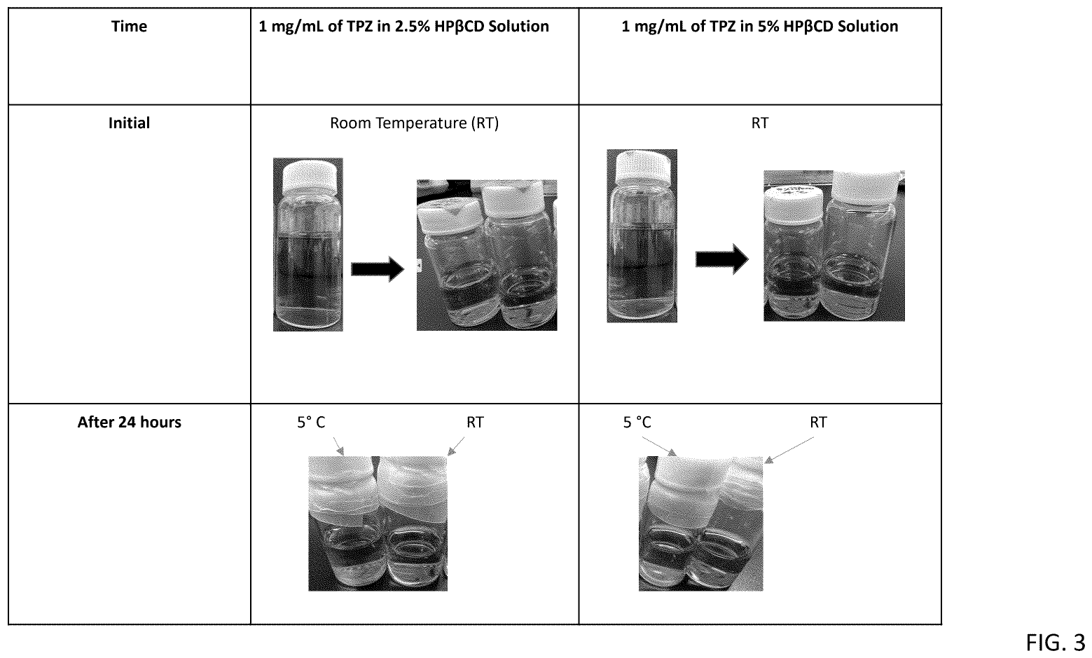

The appearance of 1 mg/ml TPZ in 5% and 2.5% HPβCD solutions is shown in FIG. 3.

TPZ can be dissolved in 5% and 2.5% HPβCD Solutions without difficulty. The 1 mg/mL of TPZ in the 5% and in 2.5% HPβCD solutions had the appearance of clear solutions. The solutions were stable for at least 24 hours at 5° C. and room temperature without forming crystal precipitates.

### Example 2: Sulfobutylether-β-Cyclodextrin (SBEβCD) as the Excipient for Tirapazamine Formulation

### Method: Solubility of Tirapazamine in 5%, 10%, 15%, 20% and 25% SBEβCD:

1. Five different concentrations (5%, 10%, 15%, 20% and 25%) of SBEPCD solutions were prepared by dissolving in water and passed through the 0.2 m PES filter.
2. Approximately 10 mg of TPZ were weighed into a glass vial.
3. Small amount of chosen SBEβCD solution was added to the vial, and the mixture was then vortexed vigorously for 1-3 minutes (and may ultrasonic mixed for 5-10 minutes) at room temperature.
4. The mixture was observed by visual if TPZ dissolves in SBEβCD solution.
5. If TPZ did not dissolve, then more SBEβCD solution was added and Example 2, steps 3-5 were repeated until TPZ was completely dissolved.
6. Following the above steps to determine the solubility of TPZ in different concentrations of SBEβCD solution.
7. The TPZ/SBEβCD solutions were stored at room temperature away from light and observed to see if TPZ precipitated out.
8. More of the SBEβCD solution (0.5 mL) was added to the TPZ/SBEβCD solutions in which TPZ precipitated out; the solutions were vortexed vigorously for 1-3 min (and may be ultrasonic mixed for 5-10 min) at room temperature, then the above step was repeated until the precipitate was completely dissolved.
9. The pH value of the final solutions was measured.
10. TPZ/SBEβCD solutions were stored at room temperature for 24 h and observed to assess stability.

### Results

### Solubility of Tirapazamine in 5%, 10%, 15%, 20% and 25% SBEβCD:

The solubility of TPZ in different concentration SBEβCD solutions at initial time are listed in Table 11.

TABLE 11
Solubility of TPZ in different concentration
SBEβCD solutions at the initial time.

| | | Vol. of | |
|---|---|---|---|
| Test | | SBEβCD | Solubility, mg/mL |
| Sample | TPZ, mg | soln., mL | (initial) |
| TPZ in 25% | 10.19 | 5 | ~2.04 |
| SBEβCD Soln. | | | |
| TPZ in 20% | 10.06 | 5 | ~2.01 |
| SBEβCD Soln. | | | |
| TPZ in 15% | 10.03 | 5 | ~2.01 |
| SBEβCD Soln. | | | |
| TPZ in 10% | 10.07 | 6 | ~1.68 |
| SBEβCD Soln. | | | |
| TPZ in 5% | 10.10 | 6 | ~1.68 |
| SBEβCD Soln. | | | |

All the TPZ/SBEβCD solutions were clear solutions initially and there was no change in the first 3 hours. After 14 hours, all the TPZ/SBEβCD solutions exhibited precipitates (needle-shaped crystals) at room temperature and could not be re-dissolved by vortex mixing.

The precipitates could be dissolved by adding more of SBEβCD solution, and the TPZ/SBEβCD solutions became clear. The appearance of each of the TPZ/SBEβCD solutions was homogenous and no precipitation was observed after stored at room temperature for 1, 24 and 48 h.

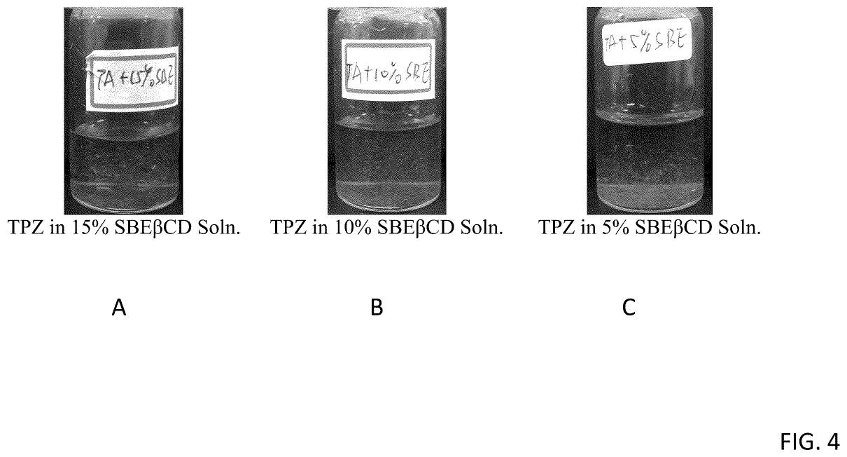

The final solubility and pH values of TPZ in 5% and 10% SBEβCD solutions are shown in Table 12. The appearance of TPZ in the SBEβCD solutions after 14 hours at room temperature is shown in FIG. 4.

TABLE 12
Final solubility and pH values of TPZ in
different concentration SBEβCD solutions.

| | | Vol. of | | |
|---|---|---|---|---|
| Test | | HPβCD | Solubility, | |
| Sample | TPZ, mg | soln., mL | mg/mL | pH value |
| TPZ in 25% | 10.19 | 5 + 0.5 = 5.5 | ~1.85 | 4.45 |
| SBEβCD Soln. | | | | |
| TPZ in 20% | 10.06 | 5 + 1 = 6 | ~1.68 | 4.85 |
| SBEβCD Soln. | | | | |
| TPZ in 15% | 10.03 | 5 + 1 = 6 | ~1.67 | 4.85 |
| SBEβCD Soln. | | | | |
| TPZ in 10% | 10.07 | 6 + 1.5 = 7.5 | ~1.34 | 4.86 |
| SBEβCD Soln. | | | | |
| TPZ in 5% | 10.10 | 6 + 1.5 = 7.5 | ~1.35 | 5.22 |
| SBEβCD Soln. | | | | |

### Example 3: Benzyl Alcohol or Nicotinamide as the Excipients for Tirapazamine Formulation Methods

### Solubility of Tirapazamine in the Presence of Benzyl Alcohol:

10 mg of Tirapazamine and 10 mg of benzyl alcohol are made to 1 mL, if not a clear solution, to 5 mL and then 10 mL of water.
1. 10 mg of TPZ and 10 mg of benzyl alcohol were added to 1 mL of water for injection (WFI) in a glass vial, and the mixture was vortexed at room temperature.
2. The solution was observed by visual inspection in order to assess whether it is clear.
3. If TPZ didn't dissolve, 4 mL of WFI was added to the solution and the mixture vortexed vigorously. This step was repeated until the solution was clear or the final volume was added to 10 mL.
4. The pH value of the final solution was measured.

### Solubility of Tirapazamine in the Presence of Nicotinamide:

10 mg of Tirapazamine and 10 mg of nicotinamide are made to 1 ml, if not a clear solution, to 5 mL and 10 ml of water.
1. 10 mg of TPZ and 10 mg of nicotinamide were added to 1 mL of WFI in a glass vial, and the mixture was vortexed at room temperature.
2. The solution was observed by visual inspection in order to assess whether it is clear.
3. If TPZ didn't dissolve, 4 mL of WFI was added to the solution and the mixture vortexed vigorously. This step was repeated until the solution was clear or the final volume was added to 10 mL.
4. The pH value of the final solution was measured.

### Results

Results of the solubility of TPZ with benzyl alcohol or TPZ with nicotinamide in WFI are listed in Table 13.

TABLE 13
Solubility and pH values of TPZ in the presence
of benzyl alcohol or nicotinamide

| | | Solubility, | |
|---|---|---|---|
| Test Sample | Vol. of WFI, mL | mg/mL | pH value |
| TPZ/Benzyl alcohol = 1/1 = | 1 + 4 + 5 = 10 | 1 | 5.66 |
| 10.04/10.55 mg | | | |
| TPZ/Nicotinamide = 1/1 = | 1 + 4 + 5 = 10 | 1 | 6.39 |
| 10.07/10.13 mg | | | |

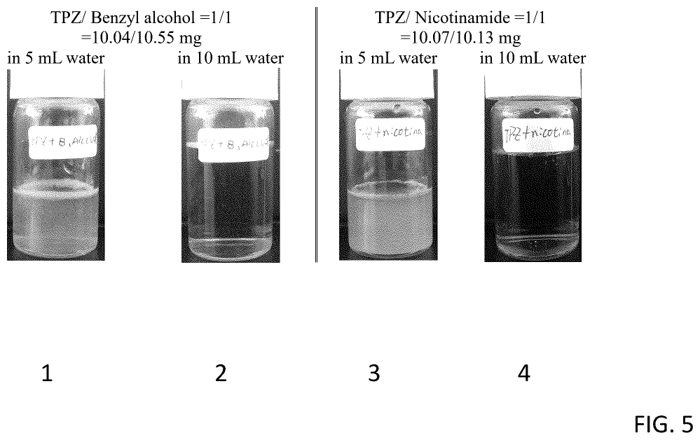

All of the TPZ solutions were clear solutions and without any change at room temperature for 24 hours. The appearance of the TPZ/BA or TPZ/Nicotinamide solutions in WFI is presented in FIG. 5.

### Example 4: Arginine or Meglumine as the Excipient for Tirapazamine Formulation

Arginine (C₆H₁₄N₄O₂) (molecular weight 174.20 g/mol) can be considered to be a strongly basic amino acid. It has three dissociation constants: the pK of the alpha carboxylic group is 2.18; the pK of the alpha amino group is 9.09; and the pK of the guanidinium group is 12.48-13.2. The guanidinium group is positively charged in neutral, acidic and even most basic environments [Retrieved Mar. 11, 2024, from https://pubchem.ncbi.nlm.nih.gov/compound/Arginine].

Meglumine (C₇H₁₇NO₅) (molecular weight 195.21 g/mol)(1-deoxy-1-(methylamino)-D-glucitol) is a hexosamine and a secondary amino compound. It is a crystalline base. [Retrieved Mar. 11, 2024, from https://pubchem.ncbi.nlm.nih.gov/compound/Meglumine].

### Solubility of Tirapazamine in the Presence of Arginine:

10 mg of Tirapazamine and 10 mg of arginine are made to 1 ml, if not a clear solution, to 5 mL and then 10 ml of water.
1. 10 mg of TPZ and 10 mg of arginine were added to 1 mL water in a glass vial, and the mixture was vortexed at room temperature.
2. The solution was observed by visual inspection in order to assess whether it was clear.
3. If TPZ didn't dissolve, 4 mL water was added to the solution and the mixture vortexed vigorously. This step was repeated until the solution was clear or the final volume was added to 10 mL.
4. The pH value of the final solution was measured and adjusted to pH 5-7.

### Solubility of Tirapazamine in the Presence of Meglumine:

10 mg of Tirapazamine and 10 mg of meglumine are made to 1 ml, if not a clear solution, to 5 mL and then 10 ml of water.
1. 10 mg of TPZ and 10 mg of meglumine were added to 1 mL of water in a glass vial, and the mixture was vortexed at room temperature.
2. The solution was observed by visual inspection to assess whether it was clear.
3. If TPZ didn't dissolve, 4 mL of water was added in the solution and the mixture vortexed vigorously. This step was repeated until the solution was clear or the final volume was added to 10 mL.
4. The pH value of the final solution was measured and adjusted to pH 5-7.

### Result

The TPZ/arginine solution and TPZ/meglumine solution with adjusted pH or not were clear solutions. No precipitate was seen after storage at room temperature for 48 hours (Table 14).

TABLE 14
Solubility and pH values of TPZ in the presence of arginine or meglumine.

| | Vol. of WFI, | Solubility, | pH value | |
|---|---|---|---|---|
| Test Sample | mL | mg/mL | (Original) | Adjust pH to 5-7 |
| TPZ/Arginine = 1/1 = | 1 + 4 + 5 = 10 | 1.0 | 10.13 | +0.46 mL of 0.1N HCl |
| 10.00/10.17 mg | | | | (→ pH = 6.88) |
| TPZ/Meglumine = 1/1 = | 1 + 4 + 3.5 = 8.5 | 1.18 | 10.41 | +0.44 mL of 0.1N HCl |
| 10.05/10.06 mg | | | | (→ pH = 6.5) |

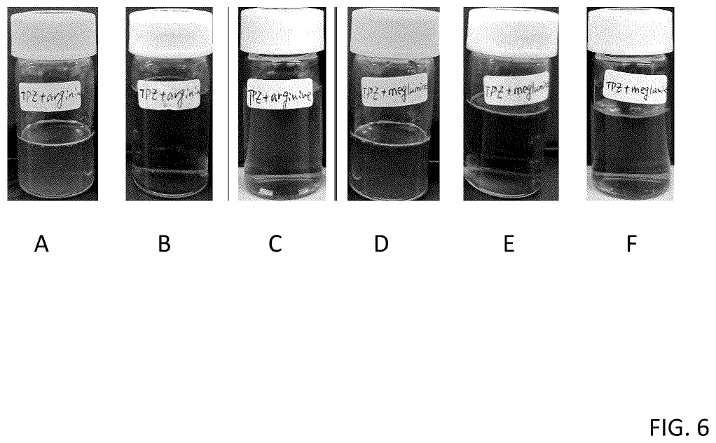

The appearance of the TPZ/arginine and TPZ/meglumine solutions in WFI are shown in FIG. 6.

### Conclusion

Of the six complexing agents evaluated, both HPβCD and SBEβCD increase the solubility of tirapazamine, and HPβCD provides the required solubility near neutral pH (4). Table 15 below summarizes TPZ solubility at various HPβCD concentrations. TPZ solubility, pH and HPβCD concentration do show strong correlations.

TABLE 15
Solubility and measured pH of TPZ at various
HPβCD concentrations at room temperature.

| Test Sample | Solubility (mg/mL) | pH |
|---|---|---|
| TPZ in 25% solution | 2.55 | 5.28 |
| TPZ in 20% solution | 2.23 | 5.44 |
| TPZ in 15% solution | 2.00 | 5.61 |
| TPZ in 10% solution | 1.69. | 5.98 |
| TPZ in 5% solution | 1.44 | 6.2 |

Unlike conventional solid oral formulations that require Drug-CD inclusion complexes to be prepared by various techniques, the HPβCD solution seems to readily accommodate TPZ in its cavity, forming TPZ-HPβCD complexes and enhancing the solubility of TPZ.

To further assess the correlations, Table 15 is replotted using the molar ratio of HPβCD/TPZ as the X axis and solubility (mg/ml) on the Y axis. FIG. 7 shows the correlation between TPZ solubility and molar ratio. It shows a strong linear relationship between solubility and the molar ratio of HPβCD/TPZ. Point A is arrived at by extrapolating the solubility to 1 mg/mL at a molar ratio of 2 (FIG. 7, point A). Point A is at about 1% HPβCD solution when converted back to common experimental conditions.

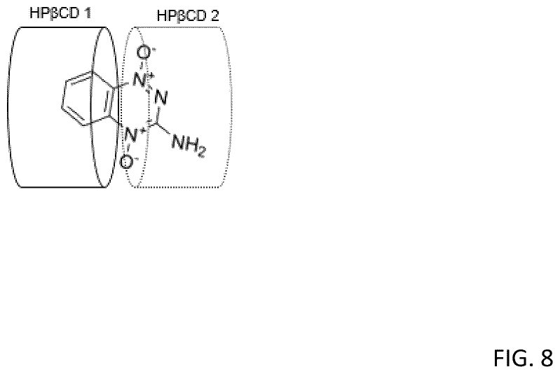

There are two main factors involved in forming an inclusion complex: (1) molecular size; and (2) an availability of a suitable linear or cyclic moiety in the molecule, which is capable of penetrating into the cavity. HPβCD and TPZ fulfill both requirements. First, with a molecular weight (MW) of 1380, HPβCD is big enough to accommodate TPZ (MW 178) in its cavity. Second, TPZ is an aromatic heterocycle di-N-oxide, which is flat, with a cyclic moiety that can penetrate into the HPβCD cavity. Without being limited by theory, it is hypothesized that the TPZ-HPβCD inclusion complex at 1 mg/ml could have a structure like that shown in FIG. 8.

The correlation between pH and the molar ratio has also been assessed. FIG. 9 is a plot of pH on the Y axis with molar ratio of HPβCD/TPZ on the X-axis with data from Table 15. FIG. 9 also shows a strong linear relationship between pH and the molar ratio of HPβCD/TPZ. Extrapolating the molar ratio to 2 arrives at a pH of 7. It suggests that the pH value of a 1% HPβCD/TPZ solution (at a molar ratio of 2) is 7.

The TPZ IV formulation of TPZ in a 1% HPβCD solution therefore provides an improved solubility of 1 mg/ml and a less irritating neutral pH environment.

### Calculation of Activation Energy for the Complex Based on Stability Studies.

Activation energy (Ea) is the least possible energy required to start a chemical reaction. Molecules need some kinetic energy or velocity to collide with other molecules to start a reaction. No reaction will take place if the collision doesn't happen, or if the molecules don't have enough kinetic energy. The energy needed to initiate the reaction is known as Activation energy.

The activation energy (Ea) of a reaction is measured in joules (J), kilojoules per mole (kJ/mol) or kilocalories per mole (kcal/mol)

The Activation Energy Formula is as follows:

$$ln k = ln A - \frac{{E}_{a}}{R} \frac{1}{T} .$$

Where A is a constant, k is the rate constant, Eₐ is the activation energy of degradation in kcal/mol, R is the universal gast constant (1.98 calKmole) and I is the absolute temperature (° K).

In the formulation without HP-beta-CD, Eₐ of TPZ is calculated as 35 kcal/mole based on the long-term stability results. For the HP-beta-CD new formulation, Eₐ of the complex is calculated at 44 kcal/mole using a new formulation methodology called “Accelerated Stability Assessment Program (ASAP)” that can be executed in a few weeks with a few storage conditions and temperature ranging from 40° C. to 70° C. inclusive. The higher the activation energy, the more kinetically stable the complex is. Hence with formation of the TPZ-HP-beta-CD complex, HP-beta-CD leads to some degree of stabilization of TPZ.

### Example 5. Hepatic Artery Ligation in a Murine Spontaneous Orthotopic Hepatocellular Carcinoma Model

Study objective: The objective of this study was to evaluate the antitumor effect of tirapazamine in a murine spontaneous orthotopic hepatocellular carcinoma model in C57BL/6Smoc-lgs2^{em1(CAG-LSL-Myc)Smoc} mice.

This study is a non-GLP study. All procedures related to animal handling, care and the treatment in this study will be performed according to guidelines approved by the Institutional Animal Care and Use Committee (IACUC) of Pharmaron following the guidance of the Association for Assessment and Accreditation of Laboratory Animal Care (AAALAC). In addition, all portions of this study were performed at Pharmaron according to the study protocol provided or approved by the Sponsor and applicable standard operating procedures.

### Study Design

### 1.1 Animals

1.1.1 Species and strain: C57BL/6Smoc-lgs2^{em1(CAG-LSL-Myc)Smoc} mice
1.1.2 Supplier: Shanghai Model Organisms Center, Inc.
1.1.3 Age: 4 to 6 weeks
1.1.4 Total number: 12 mice
1.1.5 Sex: Female

### 1.2 Animal Maintenance

1.2.1 Quarantine: Animals were quarantined for three days before the study. The general health of the animals was evaluated by a veterinarian, and complete health checks were performed. Animals with abnormalities were excluded prior to the study.
1.2.2 Housing: General procedures for animal care and housing were in accordance with the standard, Commission on Life Sciences, National Research Council, Standard Operating procedures (SOPs) of Pharmaron, Inc. The mice were kept in laminar flow rooms at constant temperature and humidity with two animals in each cage. Animals were housed in a polycarbonate cage (300×180×150 mm³) and in an environmentally monitored, well-ventilated room maintained at a temperature of 22±3° C. and a relative humidity of 50%-70%. Fluorescent lighting provided illumination approximately 12 hours per day. The bedding material was corncob, which was changed once per week.
1.2.3 Animal ID: Each animal was assigned an identification number. Each cage card was labeled with study number, group, sex, dose, animal number, initiation date, study director and telephone number. Each individual animal was identified by ear coding.
1.2.4 Diet: Animals had free access to irradiation sterilized dry granule food during the entire study period except the periods specified by the protocol.
1.2.5 Water: Sterile drinking water in a bottle was available to all animals ad libitum during the quarantine and study periods. The bottle and stopper with attached sipper tube were autoclaved prior to use. Samples of water from the animal facility were analyzed; results of water analysis were retained in the facility records and reviewed by the veterinarian or a designee to assure that no known contaminants were present that could interfere with or affect the outcome of studies.

### 1.3 Groups and Treatment

Groups and treatment were started when the orthotopic liver tumor could be palpated. The study groups and number of animals per group are shown in Table 16. The treatments were started when tumor volume reached approximately 500 mm².

TABLE 16
Groups and treatments

| | | | | Dose | Vol | | |
|---|---|---|---|---|---|---|---|
| Group | Treatment | Vehicle | N | (mg/kg) | (mL/kg) | Route | Regimen |
| 1 | Vehicle | 1% HP- | 4 | — | 5 | i.v. | Single |
| | control | beta-CD | | | | | dose |
| | | at pH 6 | | | | | |
| 2 | Tirapazamine | 1% HP- | 4 | 3 | 5 | i.v. | Single |
| | | beta-CD | | | | | dose |
| | | at pH 6 | | | | | |
| 3 | Tirapazamine | Citrate | 1 | 3 | 5 | i.v. | Single |
| | | buffer | | | | | dose |
| | | 140 mM | | | | | |
| | | at pH 6 | | | | | |
| | | Normal saline | 3 | | | | |

### 1.4 Reagents for Vehicle

Table 17 below shows reagent information:

| Reagent | MW | | Supplier | CAS# | CAT# | Lot# |
|---|---|---|---|---|---|---|
| Citric acid | 210.14 | g/mol | Sigma- | 5949-29-1 | 3314-500G | STBK4084 |
| monohydrate | | | Aldrich | | | |
| Sodium citrate | 294.10 | g/mol | Sigma- | 6132-04-3 | C8532-500G | SLCK4724 |
| tribasic dehydrate | | | Aldrich | | | |
| HP-beta-CD | 1541.54 | g/mol | Shanghai | 128446-35-5 | S11011- | A22IS213296 |

| | |
|---|---|
| yuanye | 500G |

1.4.1 Storage condition: room temperature (RT), protect from light.

1.4.2 Safety precautions: Standard laboratory safety procedures were employed for handling the test article, i.e., dust mask, safety glasses, rubber gloves and protective lab clothing were worn.

### 1.5 Test Article

1.5.1 Supplier: TECLISON INC.

1. 5.2 Information

Table 18 below shows test article information:

| Compound ID | Amount | Received Date | Comment |
|---|---|---|---|
| Tirapazamine | 150 mg | 2023 Nov. 10 | None |
| Tirapazamine | 1 mg/mL × | 2023 Nov. 10 | In 2.5% HPβCD |
| | 10 ml × 2 | | |
| | vials | | |

1.5.3 Storage condition: RT, protect from light

1.5.4 Safety precautions: Standard laboratory safety procedures were employed for handling the test article, i.e., dust mask, safety glasses, rubber gloves and protective lab clothing were worn.

### 1.6 Formulation

Formulation particulars are shown in Table 19 below.

| | | | Dosing | |
|---|---|---|---|---|
| | | Concentration | volume | |
| Compounds | Preparation | (mg/mL) | (mL/kg) | Storage |
| Vehicle control | 1% HP-beta-CD at pH = 6 | — | 5 | 4° C. |
| Citrate buffer | (1) Dissolve 5.884 g of citric | — | 5 | 4° C. |
| 140 nM at pH = 6 | acid with water to a final volume | | | |
| | of 200 mL. | | | |
| | (2) Dissolve 8.235 g of sodium | | | |
| | citrate with water to final volume | | | |
| | of 200 mL. | | | |
| | (3) Mix 9.5 ml of the citric acid | | | |
| | solution with 105 ml of sodium | | | |
| | citrate to achieve citrate buffer | | | |
| | 140 mM at pH 6. | | | |
| Tirapazamine | Dissolve 1.2 mg of Tirapazamine | 0.6 | 5 | Prepare |
| | in 2 mL of citrate buffer 140 mM | | | fresh |
| | pH 6. Pass through 0.2 μL filter | | | |
| | to sterilize before injection. | | | |
| Tirapazamine | Dissolve 1.2 mg of Tirapazamine | 0.6 | 5 | Prepare |
| | in 2 mL of 1% HP-beta-CD at | | | fresh |
| | pH 6. Pass through 0.2 μL filter | | | |
| | to sterilize before injection. | | | |
| Tirapazamine | Dissolve 1.2 mg of Tirapazamine | 0.6 | 5 | Prepare |
| | in 2 mL of normal saline. Pass | | | fresh |
| | through 0.2 μL filter to sterilize | | | |
| | before injection. | | | |

### Experimental Method and Measurement Parameters

### 2.1 Transient Hepatic Artery Ligation

Animals were anesthetized by intramuscular (IM) injection of Zoletil™ 50 (Virbac S.A.) combined with Xylazine Hydrochloride. A midline laparotomy was performed to expose the left lobe of the liver and liver hilum to dissect the common hepatic artery for transient ligation. A suture silk was put under the dissected common hepatic artery ligation. Vehicle and tirapazamine were injected into the tail vein of each mouse before the hepatic artery ligation. After completion of the injection, the mice were subjected to common hepatic artery ligation for 40 min, and the silk ligation was subsequently untied. Animals received 5 mg/kg of Meloxicam by s.c. injection for analgesic therapy once per day before being taken down for sample collection.

### 2.2 Measurement Parameters

For routine monitoring, all study animals were monitored not only for tumor growth but also for behavior such as mobility, food and water consumption (by cage side checking only), body weight (BW), eye/hair matting and any other abnormal effect. Any mortality and/or abnormal clinical signs were recorded.

The body weight of all animals was measured daily throughout the study. The measurement date was specified as per study design. Body weight change, expressed in %, was calculated using the following formula:

BW change (%)=(BW_{Day X}/BW_{Day 0})×100, where BW_{Day X} is BW on a given day, and BW_{Day 0} is BW on Day 0 (initiation of treatment

### 2.3 Sample Collection

The liver tumor tissues were collected at 24 h post the single dose for H & E staining as shown in Table 20 below.

TABLE 20
Sample collection

| | | | | | | Liver |
|---|---|---|---|---|---|---|
| | | | Dose | | | tumor |
| Groups | Treatment | Vehicle | (mg/kg) | Timepoint | N | for HE |
| 1 | Vehicle | 1% HP- | — | 24 h | 4 | 4 |
| | control | beta-CD | | | | |
| | | at pH 6 | | | | |
| 2 | Tirapazamine | 1% HP- | 3 | 24 h | 5 | 3 |
| | | beta-CD | | | | |
| | | at pH 6 | | | | |
| 3 | Tirapazamine | Normal saline | 3 | 24 h | 6 | 3 |

| | | |
|---|---|---|
| Total | 10 | 10 |

H & E staining: 10 tumor samples.

Liver tumors were fixed in NBF (10% neutral buffered formalin) for 72 h and then transferred into 50% ethanol for H& E staining.

### 2.4 Termination Criterion

Individual animals in a continuing deteriorating condition were euthanized.

Animals showing obvious signs of severe distress and/or pain were humanely euthanized by carbon dioxide; the animal's death was ensured by cervical dislocation. In the event of the following situations, euthanasia was performed to the affected animals:
(1) animals had lost significant body mass (emaciated). Obvious body weight loss >20% and cannot recover after dosing holiday.
(2) Animals cannot get to adequate food or water.

### 2.5 Data Acquisition

Protocol-required measurements and observations were recorded manually on appropriate forms, or directly on a computerized database.

### Results

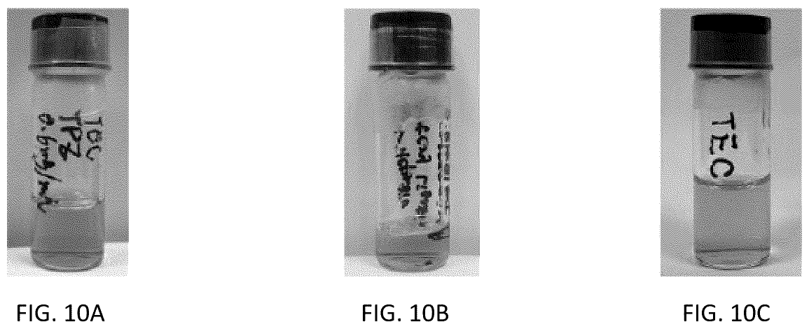

Results are shown in Table 21 below and in FIGS. 10-22.

Table 21 shows body weight and treatment protocols comprising hepatic artery ligation (HAL) for groups 1, 2 and 3 (a and b).

| | | Body | Dosing & | | | |
|---|---|---|---|---|---|---|
| | Animal | weight* | HAL | Sampling | Tumor | |
| Groups | ID | (g) | Date | Date | FFPE** | Comment |
| Group 1 | 9 | 24.4 | Feb. 22, 2024 | Feb. 23, 2024 | 1 | |
| Vehicle control (1% | 10 | 20.7 | Feb. 26, 2024 | Feb. 27, 2024 | 1 | |
| HP-beta-CD at pH 6) | 11 | 21.3 | Feb. 26, 2024 | Feb. 27, 2024 | 1 | |
| single dose, i.v. | 12 | 23.0 | Feb. 26, 2024 | Feb. 27, 2024 | 1 | |
| | Mean | 22.4 | | | | |
| | SD | 1.7 | | | | |
| Group 2 | 1 | 20.3 | Jan. 9, 20224 | Jan. 10, 2024 | 1 | Animal |
| Tirapazamine 3 mg/kg | | | | | | was |
| Vehicle: (1% HP-beta- | | | | | | moribund |
| CD at pH 6) | | | | | | before |
| Single dose, i.v. | | | | | | sample |
| | | | | | | collection |
| | 2 | 22.1 | Jan. 19, 2024 | Jan. 20, 2024 | 1 | |
| | 3 | 22.3 | Jan. 19, 2024 | — | — | Found dead |
| | | | | | | on |
| | | | | | | Jan. 20, 2024 |
| | 4 | 22.3 | Jan. 26, 2024 | Jan. 27, 2024 | 1 | Animal |
| | | | | | | was |
| | | | | | | moribund |
| | | | | | | before |
| | | | | | | sample |
| | | | | | | collection |
| | Mean | 21.8 | | | | |
| | SD | 1.0 | | | | |
| Group 3 | 5 | 21.2 | Feb. 6, 2024 | — | — | Died after |
| Tirapazamine 3 mg/kg | | | | | | dosing on |
| (vehicle: Citrate buffer | | | | | | Feb. 6, 2024 |
| 140 mM at pH 6) #5 | 6 | 23.4 | Feb. 19, 2024 | Feb. 20, 2024 | 1 | |
| (3a) | 7 | 23.8 | Feb. 19, 2024 | Feb. 20, 2024 | 1 | |
| Tirapazamine 3 mg/kg | 8 | 21.6 | Feb. 22, 2024 | Feb. 23, 2024 | 1 | |
| (vehicle: normal saline | | | | | | |
| (#6-#8)(3b) | | | | | | |
| Single dose, i.v. | | | | | | |
| | Mean | 22.5 | | | | |
| | SD | 1.3 | | | | |

*Body weight is measured on the closing day.
**FFPE = formalin fixed paraffin embedded tissue sections of the liver tissue comprising tumor stained with H&E.

H & E staining of the liver tumors. The goal of this animal study was to examine th effect of the new TPZ HPβCD formulation to induce liver tumor necrosis when combined with HAL, which was used to recapitulate the clinical Trans-Arterial Embolization (TAE). A mouse model of HCC that developed spontaneously in mice with a genetic background C57/BL/6Smoc-Igs2^{em1(CAG-LSL-Myc)Smoc} was used. The study was designed as a tumor killing model instead of a tumor growth suppression model. Hence, mice were treated only when a liver tumor could be palpated before undergoing treatment. Due to a high mortality after the surgical intervention by HAL, mice were euthanized at the second day after treatment and the liver tumor was harvested for H&E staining to check for the presence of necrosis. Initially TPZ in citrate buffer (pH=4) was used, which was the prior formulation used in clinical trials. However, the first mouse injected with this acidic buffer died immediately after injection. This prompted an amendment of the study protocol to use normal saline as the alternative vehicle of TPZ for subsequent experiments in that group. Tumor necrosis from all three groups was evaluated by H&E staining after mice were euthanized and tumor harvested.

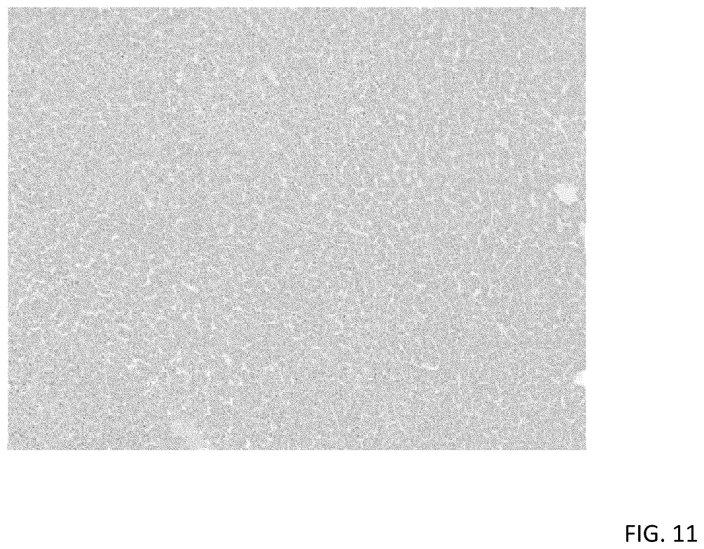

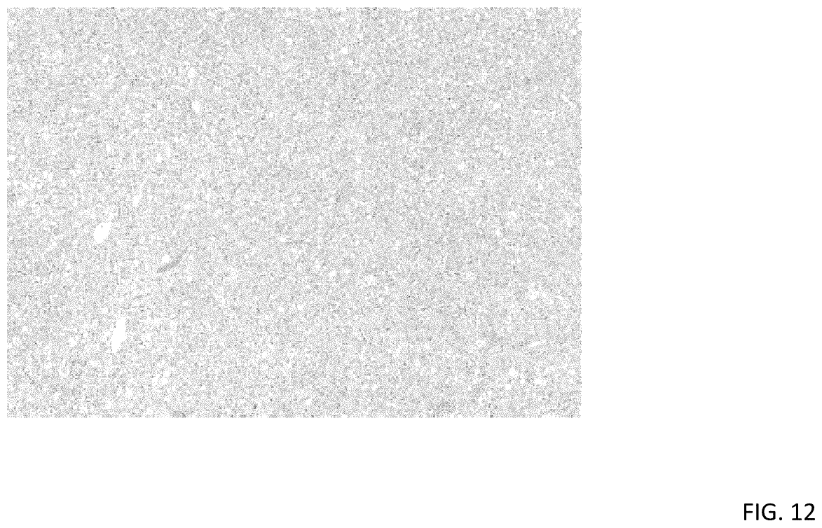

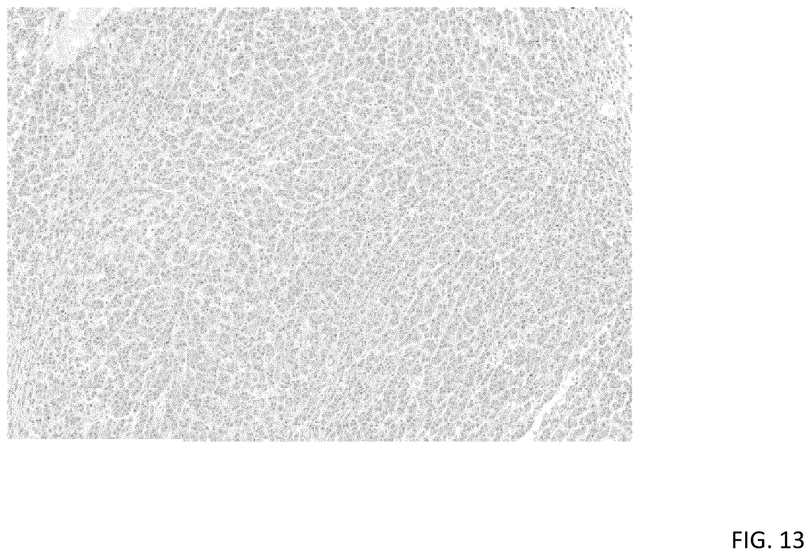

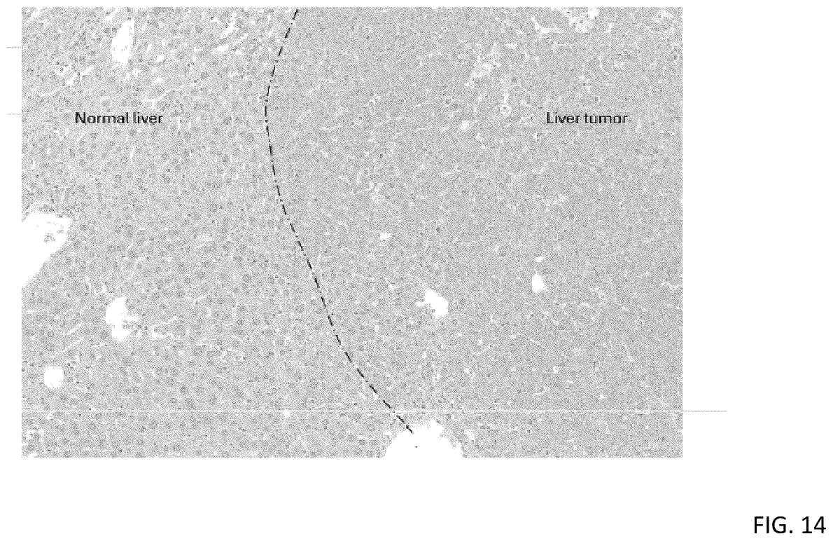

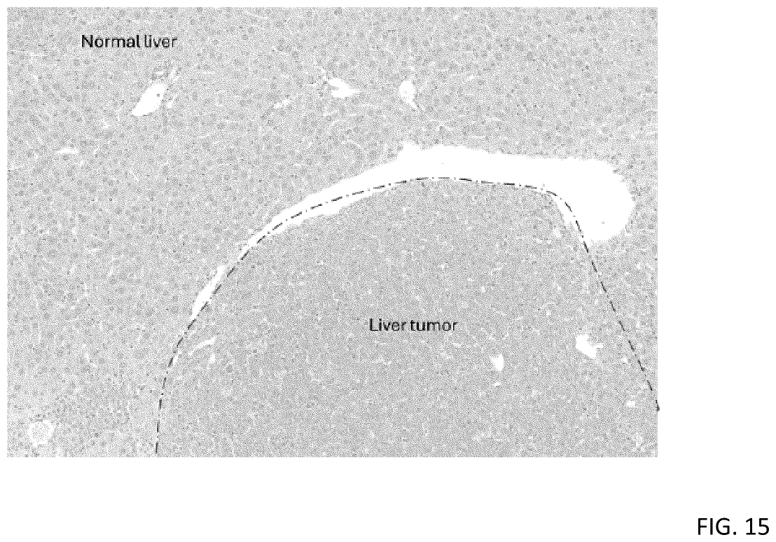

From FIG. 11, FIG. 12, FIG. 13, FIG. 14 and FIG. 15, there was no evidence of necrosis either in tumor or normal liver parenchyma in mice treated with 1% HPβCD alone without TPZ, which confirmed the non-toxic nature of 1% HPβCD.

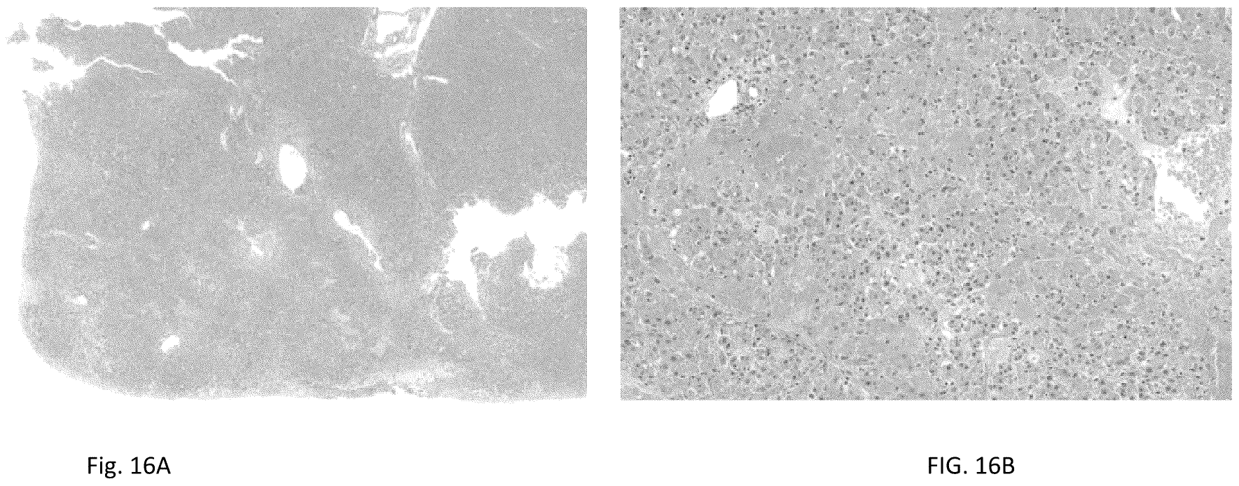

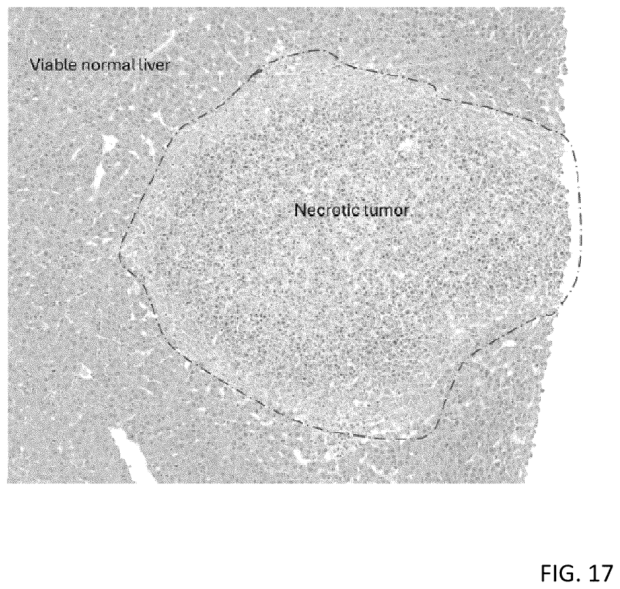

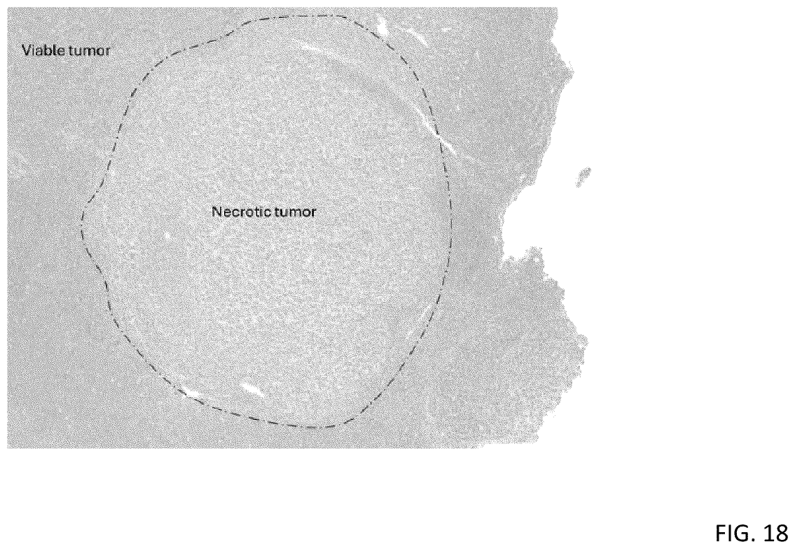

FIG. 16, FIG. 17 and FIG. 18 show tumors from the Group #2 (n=3) animals treated with TPZ in 1% HPβCD.

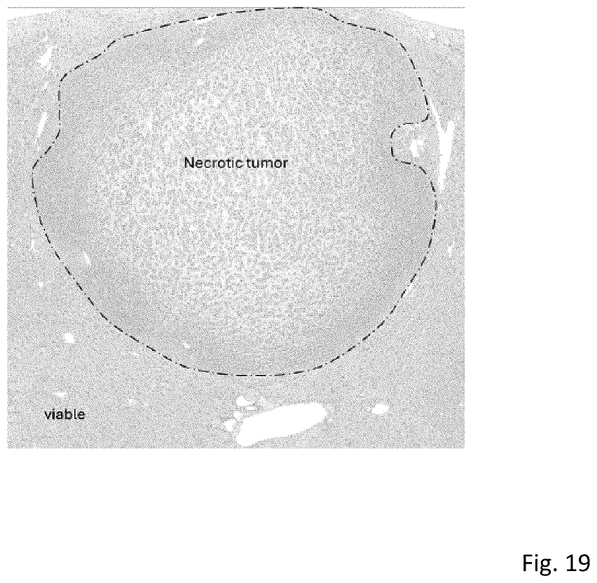

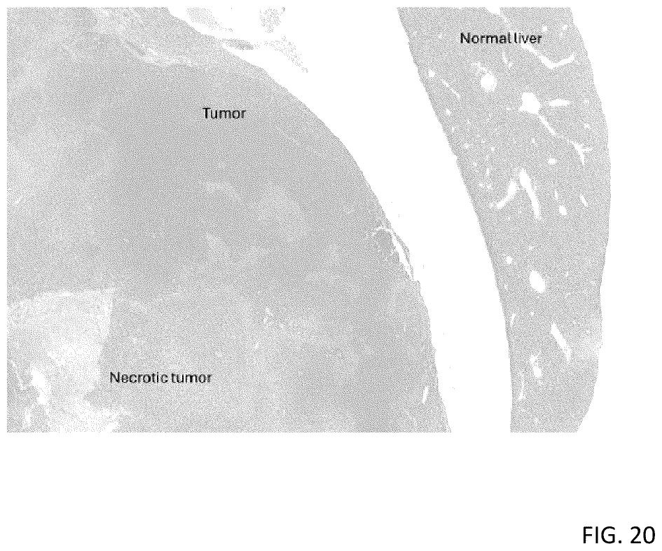

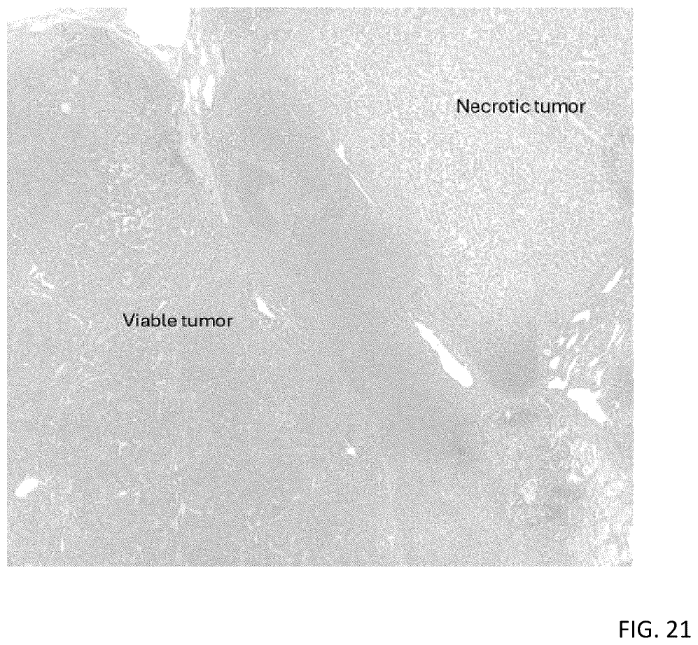

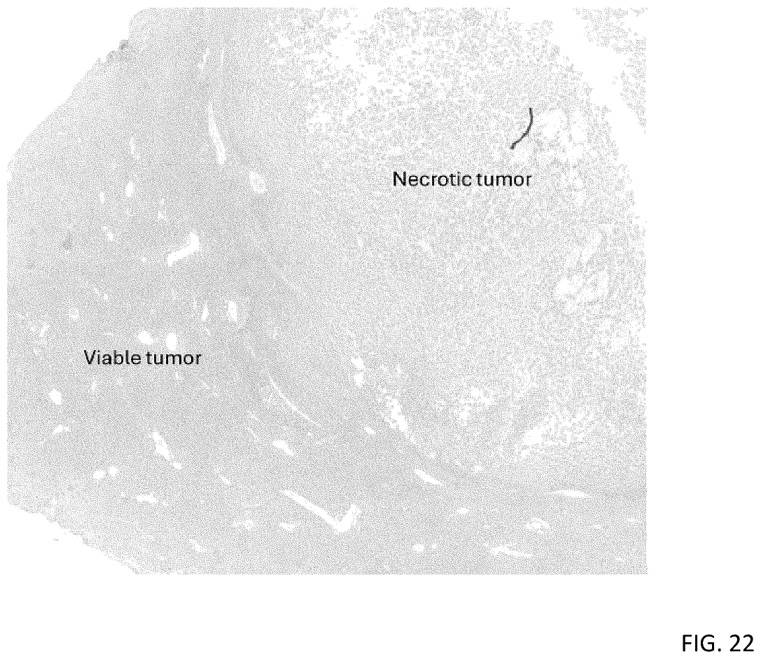

FIG. 19, FIG. 20, FIG. 21, and FIG. 22 show tumors from Group #3 (n=3) animals treated with TPZ in normal saline. In all liver tumor lesions from Groups #2 and 3, liver tumors exhibited areas of necrosis.

### Example 6. PK Analysis of Tirapazamine (TPZ) and its Metabolites, SR4317 and SR4330 after Intra-Arterial Injection of Tirapazamine in NK vx. HPβCD in Sprague Dawley Rats

Purpose of the study: To evaluate the pharmacokinetics (PK) of tirapazamine and its metabolites, SR4317 and SR4330 [Graham, M A et al. Cancer Chemother. Pharmacol. (1997) 40 (1): 1-10], after injection of TPZ in 1% HPβCD or normal saline.

Study site: Rutgers University animal facility was contracted for the study. They provided an IACUS approved animal use protocol and executed these studies per protocol. The animal facility purchased surgically modified rats from a vendor approved by Rutgers University. Primera Analytic Solutions Corp. in Princeton, NJ conducted the PK sample analysis.

Test Materials: Teclison Inc. provided the experimental compound tirapazamine in solutions ready to be used.

Animals: Twenty-four female (12) and male (12) Sprague Daley (SD) rats, weight 200-250 grams, respectively, with femoral artery and jugular vein cannulated, were purchased from, Taconic. Two extra cannulated male and female rates were used to test tolerability with buffer. Each rate was housed individually to safeguard the vessel catheters.

Two test rats were first injected with citrate buffer (pH=4) only (over 4 min) to make sure that the rats could tolerate 2.5 mL citrate buffer (pH=4) without immediate death within 1 hour. However, the first rat died with injection of 2.5 mL citrate buffer, the second rat died after injection with 1.5 mL citrate buffer (over 4 min). No blood sample collection occurred for these two test rats.

### Study Procedure

The study parameters are shown in Table 22. began after a 3-day period of adjustment to the animal facility. The study compound was injected into the femoral artery catheter (FAC at 2-1 m (by slow injection. Blood samples were collected from a jugular vein catheter (JVC) at 6 timepoints: 0 (baseline) and after drug administration at 15 and 30 minutes, 1 hr, 2 hr, and 6 hours. The volume of blood sample for each time point was approximately 0.6 mL. Plasma samples were collected in tubes containing sodium heparin as anti-coagulant. Plasma was prepared and frozen in −80° C. Rats were euthanized after the last blood sample (euthanized after the 6 hr time point).

The doses received by each group of rats are as follows:

Male rats were around 250 grams, and female 200 grams. There were 8 groups with Groups 1-4 females, and Groups 5-7 males. Each group had 3 rats. TPZ was dissolved in NS or 10 HPβCD at a concentration 0.7 mg/mL.

TABLE 22

| | Animal #/sex | | Volume of TPZ | | |
|---|---|---|---|---|---|
| | (weight in | | (0.7 mg/mL) | | |
| Group # | grams) | Dosing, FAC Port | injection | | Bleeding, JVC port |
| 0 | 2M (250) | Citrate buffer | 2.5 | mL | None |
| 1 | 3F (200) | 3.33 mg/kg in NS | 0.67 | mL | 0, 15, 30 min, 1, 2, 6 hr |
| 2 | 3F (200) | 3.33 mg/kg in | 0.67 | mL | 0, 15, 30 min, 1, 2, 6 hr |
| | | HPβCD | | | |
| 3 | 3F(200) | 7 mg/kg in NS | 2.0 | mL | 0, 15, 30 min, 1, 2, 6 hr |
| 4 | 3F (200) | 7 mg/kg in HPβCD | 2.0 | mL | 0, 15, 30 min, 1, 2, 6 hr |
| 5 | 3M (250) | 3.33 mg/kg in NS | 0.83 | mL | 0, 15, 30 min, 1, 2, 6 hr |
| 6 | 3M (250) | 3.33 mg/kg in | 0.83 | mL | 0, 15, 30 min, 1, 2, 6 hr |
| | | HPβCD | | | |
| 7 | 3M (250) | 7 mg/kg in NS | 2.5 | mL | 0, 15, 30 min, 1, 2, 6 hr |
| 8 | 3M (250) | 7 mg/kg in HPβCD | 2.5 | mL | 0, 15, 30 min, 1, 2, 6 hr |

The concentrations of TPZ and its metabolites, SR4317 and SR4330, in rat plasma samples were analyzed using liquid chromatography Tandem Mass Spectrometry (LC-MS) by Primera Analytical Solutions Corp (Princeton, NJ). Standards for TPZ, SR4317 and SR4330 were provided by Teclison, Inc. (Princeton, NJ) for calibration. All analytical data were reviewed for completeness and accuracy. Data transferred manually was cross-checked against source data which was part of the study raw data.

LC-MS analysis was conducted with Sciex 4000 with a Shimadzu LC-30 System.

The concentration data was imported and plotted with SAS program as ADPC (Analysis Dataset of Pharmacokinetics Concentrations) dataset. PK parameters calculated based on ADPC were output as an Analysis Dataset of Pharmacokinetics Parameters (ADPP) dataset.

PK parameters of each profile were calculated by non-compartmental analysis using SAS programming. BLQ (Below the Limit of Quantification) observation prior to the first measurable concentration was imputed as zero, otherwise, the BLQ observation was left as missing.

An apparent first-order terminal elimination rate constant (Kel) was calculated by linear least-squares regression analysis using at least three points (excluding Cₘₐₓ) in the terminal log-linear phase. Uniform weighting was adopted when performing regression analysis. Linear-up/log-down approach was utilized to calculate AUC.

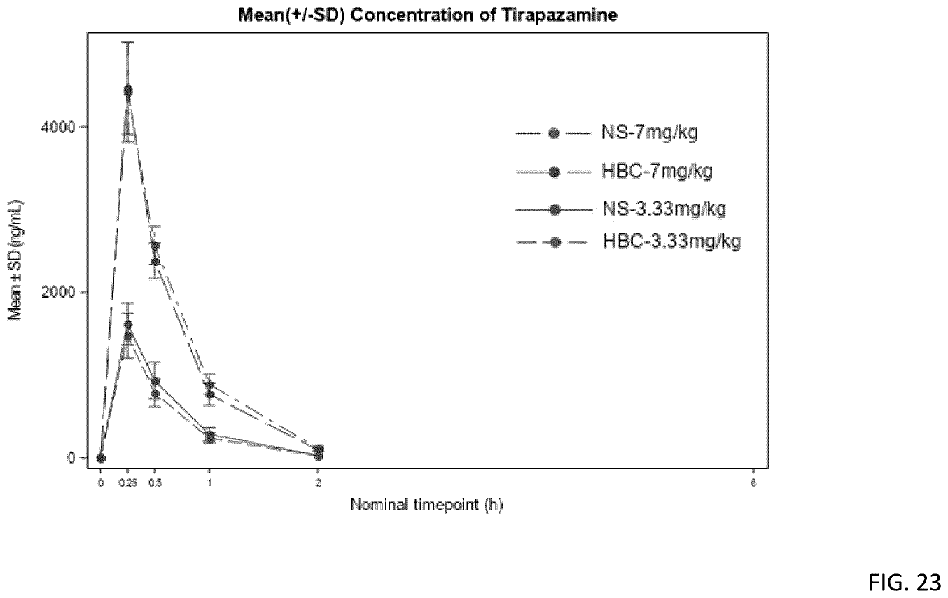

FIG. 23 shows the plasma concentrations of TPZ plotted against nominal time point (hour) for the 4 groups after injection of TPZ in either NS or 1% HPβCD. A dose proportional effect was observed in 3.33 mg/kg vs. 7 mg/kg in either solvent. Dose proportionality occurs when increases in the administered dose are accompanied by proportional increases in a measure of exposure like AUC or Cₘₐₓ. There was no difference between the groups that received TPZ in NS or HPβCD.

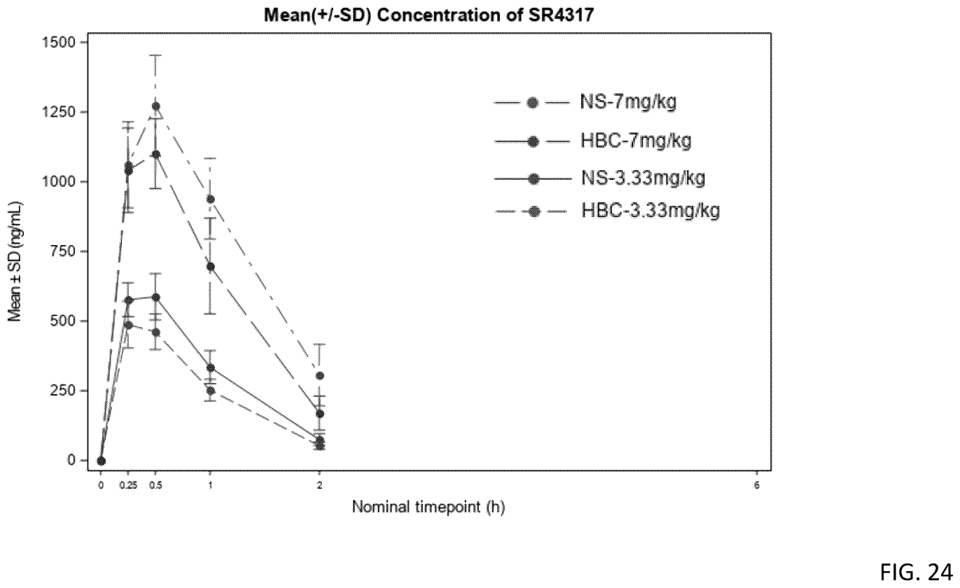

FIG. 24 shows the plasma concentrations of SR4317, a metabolite of TPZ with one fewer oxygen atom from TPZ, plotted against nominal time point (hour) for the 4 groups after injection of TPZ in NS or 1% HPβCD. A dose proportional effect was observed in 3.33 mg/kg vs. 7 mg/kg in either solvent. However, the plasma concentrations of SR4317 were reduced by 20-30% in the curve of HPβCD than that of NS. This result suggests that formation of the TPZ-HPβCD complex somewhat reduced the bioavailability of TPZ and resulted in a decreased production of TPZ metabolite SR4317.

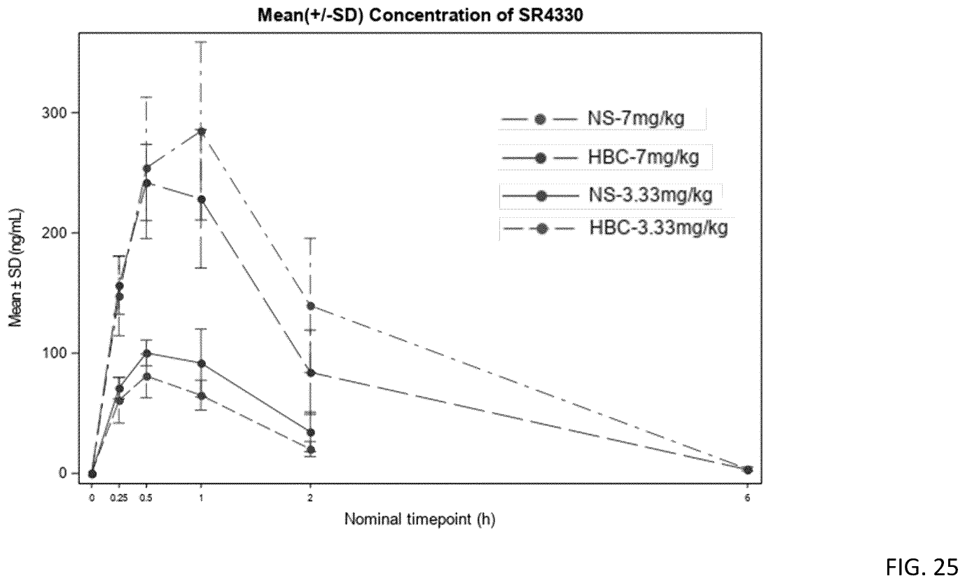

FIG. 25 shows the plasma concentrations of SR4330, the second metabolite of TPZ with two fewer oxygen atoms from TPZ, plotted against nominal time point (hour) for the 4 groups after injection of TPZ in NS or 1% HPβCD. A dose proportional effect was observed in 3.33 mg/kg vs. 7 mg/kg in either solvent. The half-life of SR4330 was longer than TPZ and SR4317. The plasma concentrations of SR4330 were reduced by 20-30% in the curve of HPβCD than that of NS. This result is consistent with the previous result of SR4317 and supports that formation of TPZ-HPβCD complex reduced the bioavailability of TPZ and resulted in a decreased production of TPZ metabolite SR4330.

The key parameters of TPZ and its two metabolites are tabulated in Table 23 below:

| | | | | | | | HβC/NS |
|---|---|---|---|---|---|---|---|
| | | Dosage | | | Normal Saline (NS) | | Buffer |
| Analyte | Parameter | (mg/kg) | HβC Mean (SD) | | Mean (SD) | | Ratio (%) |
| TPZ | Cₘₐₓ | 3.33 | 1476.67 | (270.53) | 1620.00 | (252.032) | 91.15% |
| | | 7 | 4468.33 | (552.573) | 4423.33 | (605.464) | 101.02% |
| | AUCₗₐₛₜ | 3.33 | 781.49 | (154.482) | 898.92 | (189.489)) | 86.94% |
| | | 7 | 2423.84 | (276.41) | 2578.02 | (269.700) | 94.02% |
| SR4317 | Cₘₐₓ | 3.33 | 490.17 | (81.732) | 608.00 | (61.751) | 80.62% |
| | | 7 | 1122.67 | (114.729) | 1273.33 | (82.450) | 88.17% |
| | AUCₗₐₛₜ | 3.33 | 479.96 | (60.658) | 614.61 | (82.450) | 78.09% |
| | | 7 | 1210.97 | (198.506) | 1535.52 | (241.431) | 78.86% |
| SR4330 | Cₘₐₓ | 3.33 | 81.88 | (17.213) | 105.45 | (18.540) | 77.65% |
| | | 7 | 254.83 | (36.706) | 285.33 | (73.576) | 89.31% |
| | AUCₗₐₛₜ | 3.33 | 99.89 | (18.653) | 136.67 | (32.723) | 73.09% |
| | | 7 | 385.93 | (114.589) | 518.92 | (185.746) | 74.37& |

A linear model was built to evaluate the effect of solution on the PK parameters, adjusting for sex and dose. The model included sex (F, M), solution type (Normal saline, Hydroxypropyl-beta-Cyclodextrin “HP-beta-CD”), and dose (mg) as fixed effects. Sex and solution type are categorical variables (meaning a variable based on a qualitative, not numerical, property) and dose is a continuous variable (meaning a random variable that can take on an infinite number of values within a certain interval that represents measurable data). Separate models were built for each combination of analyte (TPZ, SR4317 and SR4330) and PK parameters (AUCₗₐₛₜ and Cₘₐₓ) using the BY statement, which divides the observations from an input data set into groups for processing.

The difference of lease squared means between solution types was estimated. 95% confidence intervals (CI), and p-values for the difference were presented. The significance level (alpha) was set at 0.05, meaning that a p-value less than 0.05 was considered statistically significant.

The results are shown in Table 24 below. Negative differences of least squares mean in all six analyte-parameter combinations showed that the HP(3CD group had lower least squares means than that of the normal saline group. For both SR4317 Cₘₐₓ and AUCₗₐₛₜ, the 95% CI does not include 0 and p-values are <0.01, which indicates that the two solutions are statistically different.

TABLE 24

| | | Differences of Least Squares Means | |
|---|---|---|---|
| | | between HP-beta-CD and normal | |
| Analyte | Parameter | saline (95% CI) | P-value |
| TPZ | Cₘₐₓ (ng/mL) | −150.81 (−590.29, 288.66) | 0.4824 |
| | AUCₗₐₛₜ (ng · h/mL) | −193.89 (−421.89, 34.11) | 0.0913 |
| SR4317 | Cmax (ng/mL) | −156.56 (−264.96, −48.17) | 0.0069 |
| | AUCₗₐₛₜ (ng · h/mL) | −257.84 (−434.81, −80.87) | 0.0065 |
| SR4330 | Cₘₐₓ (ng/mL) | −33.19 (−73.54, 7.16) | 0.1016 |
| | AUCₗₐₛₜ (ng · h/mL) | −96.45 (−200.03, 7.13) | 0.0663 |

Based on the PK analysis, complexed TPZ-HP(3CD administered by injection is pharmacokinetically different from TPZ in NS with a reduced Cₘₐₓ and AUCₗₐₛₜ for its metabolites even with identical levels of TPZ in circulation. This result implies that the formation of a TPZ-HPβCD complex compared to free TPZ confers differences in biological characteristics that affects the pharmacokinetics of the TPZ-HPβCD in rats.

### CONCLUSIONS

This experiment was planned to examine the effectiveness of the new formulation when combined with HAL. Due to a technical difficulty and high mortality after conducting surgery and HAL in mice, the sample size was small (n=3 or 4) and did not allow a formal statistical comparison of the two groups. Nonetheless, it was confirmed that the new formulation of TPZ in 1% HPβCD was effective in causing tumor necrosis when combined with HAL, whereas mice treated with 1% HPβCD vehicle along with HAL had no evidence of liver tumor necrosis.

For the following reasons, we conclude that complexed TPZ is a patentably distinct composition/dosage form of TPZ.

First, as described in the specification, the physical characteristics of TPZ were changed by complexation of TPZ with hydroxypropyl β cyclodextrin (HPβCD). For example, complexation with HPβCD changes the solubility of TPZ such that the higher the percentage of HPβCD in the final formulation, the more soluble TPZ becomes. Second, although the pKa of TPZ is around 12, various concentrations of HPβCD also changed the pH of the TPZ solution.

Second, the PK results described in the specification arguably show that complexed TPZ is pharmacokinetically different from TPZ in NS alone and provide experimental evidence that TPZ-HPβCD and TPZ in NS are different compositions/dosage forms. Arguably, the complexed TPZ is not anticipated by TPZ in NS saline and the pharmacokinetic results obtained with the complexed TPZ are not the natural result of the combination of elements present in TPZ in NS saline. Further, the complexed TPZ is not obvious over TPZ in NS saline because the pharmacokinetic differences between the two dosage forms could not have been predicted with a reasonable likelihood of success.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1. A cyclodextrin inclusion complex comprising a β-cyclodextrin host molecule substituted by hydroxypropyl groups (hydroxypropyl-β-cyclodextrin, or HPβCD) or by sulfopropylether groups (sulfobutylether-β-cyclodextrin or SBEβCD) and comprising a cavity containing tirapazamine as a guest, wherein
the tirapazamine guest is at least partially included into the cavity of the β-cyclodextrin host molecule; wherein the extent of inclusion ranges from about 1% to about 50%, inclusive; and
a molar ratio of the cyclodextrin host to the tirapazamine guest ranges from about 14:1 to about 2:1, inclusive.

2. The cyclodextrin inclusion complex according to claim 1, wherein
the molar ratio of the β-cyclodextrin host to the tirapazamine guest in the complex is about 2:1; and
a 0.7 mg/ml solution of tirapazamine complexed in at least a 1% solution of the substituted β-cyclodextrin is water soluble.

3. The cyclodextrin inclusion complex according to claim 2, wherein pH of the 0.7 mg/mL solution of tirapazamine complexed to the β-cyclodextrin ranges from about pH 5.3 to about pH 6.4, inclusive.

4. The cyclodextrin inclusion complex according to claim 1, wherein the dissolved complex is stable for at least 24 hr when stored at 202-25° C. (room temperature) or at 5° C.

5. The cyclodextrin inclusion complex according to claim 1, wherein the β-cyclodextrin host molecule is substituted by hydroxypropyl groups (hydroxypropyl-β-cyclodextrin, or HPβCD).

6. The cyclodextrin inclusion complex according to claim 5, wherein solubility of the complexed TPZ in at least a 1% solution of the HPβCD host at room temperature ranges from about 1 mg/mL to 2.55 mg/mL, inclusive, at a pH range from about pH 5.8 to about pH 6.2, inclusive.

7. The cyclodextrin inclusion complex according to claim 6, wherein solubility of the complexed TPZ in at least the 1% solution of the HPβCD host at room temperature at a molar ratio of the β-cyclodextrin host to the tirapazamine guest of 2.0 is about 0.7-1 mg/mL, inclusive, at a pH of 6.

8. A pharmaceutical composition comprising a cyclodextrin inclusion complex comprising a β-cyclodextrin host molecule substituted by hydroxypropyl groups (hydroxypropyl-β-cyclodextrin, or HPβCD) or by sulfopropylether groups (sulfobutylether-β-cyclodextrin or SBEβCD) and comprising a cavity containing tirapazamine as a guest,
wherein
the carrier is an aqueous carrier;
the tirapazamine guest is at least partially included into the cavity of the β-cyclodextrin host molecule;
the extent of inclusion ranges from about 1% to about 50%, inclusive; and
a molar ratio of the cyclodextrin host to the tirapazamine guest ranges from about 14:1 to about 2:1, inclusive.

9. The pharmaceutical composition according to claim 7, wherein
the molar ratio of the cyclodextrin host to the tirapazamine guest is about 2:1;
the β-cyclodextrin host molecule is substituted by hydroxypropyl groups (hydroxypropyl-β-cyclodextrin, or HPβCD); and
about a 0.7-1 mg/ml solution of the complexed tirapazamine guest in at least a 1% solution of the substituted β-cyclodextrin host is water soluble.

10. The pharmaceutical composition according to claim 9, wherein pH of the solution comprising the tirapazamine guest complexed with the β-cyclodextrin host ranges from about 5.3 to about 6.4, inclusive.

11. The pharmaceutical composition according to claim 8, wherein the pharmaceutical composition comprising the complexed tirapazamine comprises improved stability at room temperature compared to the stability of non-complexed tirapazamine alone.

12. The pharmaceutical composition according to claim 8, wherein the aqueous carrier is water, normal saline, Ringer's solution or a dextrose solution.

13. The pharmaceutical composition according to claim 8, wherein the pharmaceutical composition comprising the β-cyclodextrin-complexed tirapazamine is formulated for administration intra-arterially or by intravenous infusion.

14. The pharmaceutical composition according to claim 8, wherein the pharmaceutical composition comprising the β-cyclodextrin-complexed tirapazamine comprises reduced toxicity comprising injection-related pain when compared to the toxicity of the non-complexed tirapazamine alone.

15. A method of treating a solid tumor comprising
(a) targeting the solid tumor by administering a pharmaceutical composition comprising a cyclodextrin inclusion complex comprising a β-cyclodextrin host molecule substituted by hydroxypropyl groups (hydroxypropyl-β-cyclodextrin, or HPβCD) comprising a cavity containing tirapazamine as a guest,
wherein
the carrier is an aqueous carrier;
pH of a 0.7-1 mg/mL aqueous solution of the complexed tirapazamine guest ranges from pH 5.3 to pH 6.4, inclusive;
the tirapazamine guest is at least partially included into the cavity of the β-cyclodextrin host molecule, wherein the extent of inclusion ranges from about 1% to about 50%, inclusive; and
a molar ratio of the cyclodextrin host to the tirapazamine guest ranges from about 14:1 to about 2:1, inclusive;
(b) transiently blocking arterial blood supply to the solid tumor of the subject by transarterial embolization so that the cyclodextrin inclusion complex comprising tirapazamine is transiently retained within the tissue comprising the solid tumor; and
(c) producing targeted necrosis within the solid tumor and not viable tissue.

16. The method according to claim 15, wherein the pharmaceutical composition comprising the cyclodextrin inclusion complex of tirapazamine alone reduces toxicity of injection-related pain when compared to noncomplexed tirapazamine.

17. The method according to claim 15, wherein the transient transarterial embolization is for a time period of at least about 40 minutes.

18. The method according to claim 15, wherein the administering is intravenously or intra-arterially.

19. The method according to claim 15, wherein
the molar ratio of the cyclodextrin host to the tirapazamine guest is about 2:1;
the β-cyclodextrin host molecule is substituted by hydroxypropyl groups (hydroxypropyl-β-cyclodextrin, or HPβCD); and
a 0.7-1 mg/ml solution of the tirapazamine guest complexed with at least a 1% solution of the substituted cyclodextrin host is water soluble.

20. The method according to claim 15, wherein the aqueous carrier is water, normal saline, Ringer's solution or a dextrose solution.

21. The method according to claim 15, wherein the solid tumor is a primary or metastatic carcinoma including a breast, a lung, an esophageal, a liver, a stomach, a colon, a rectum, a pancreas, a prostate, and a uterus adenocarcinoma.

22. The method according to claim 21, wherein the carcinoma suitable for transarterial embolization is a primary liver cancer or a hepatocellular carcinoma.
